(19) United States
(12) Patent Application Publication
(10) Pub. No.: US 2024/0271159 A1
(43) Pub. Date: Aug. 15, 2024

(54) AAV VECTOR COLUMN PURIFICATION METHODS

(71) Applicant: Spark Therapeutics, Inc., Philadelphia, PA (US)

(72) Inventors: Ohnmar KHANAL, Ardmore, PA (US); Vijesh KUMAR, Philadelphia, PA (US); Mi JIN, Berwyn, PA (US)

(21) Appl. No.: 18/568,530

(22) PCT Filed: Jun. 10, 2022
(86) PCT No.: PCT/US2022/072859

§ 371 Date: Dec. 8, 2023

Related U.S. Application Data
(60) Provisional application No. 63/209,680, filed on Jun. 11, 2021.
(60) Provisional application No. 63/366,094, filed on Jun. 9, 2022.

(51) Int. Cl. C12N 15/86; B01D 15/36; B01D 15/42

(52) U.S. Cl. CPC C12N 15/86; B01D 15/363; B01D 15/422; C12N 2750/14143; C12N 2750/14151

(57) ABSTRACT

Described herein is methods for rAAV particles purification, particularly for the purification of full rAAV particles from rAAV preparations comprising both the full rAAV particles and non-full particles.

### CROSS REFERENCE TO RELATED APPLICATION

This application claims priority to U.S. Provisional Patent Application Nos. 63/209,680, filed Jun. 11, 2021, and 63/366,094, filed Jun. 9, 2022, the disclosures of which are incorporated herein by reference.

### TECHNICAL FIELD

This application relates to methods for purifying recombinant adeno-associated virus (rAAV) particles. More particularly, the application relates to methods for purifying full rAAV particles from preparations comprising both full and non-full rAAV particles.

### BACKGROUND OF THE INVENTION

Gene delivery is a promising method for the treatment of acquired and inherited diseases. A number of viral-based systems for gene transfer purposes have been described, including adeno-associated virus (AAV)-based systems.

AAV is a helper-dependent DNA parvovirus that belongs to the genus Dependovirus. AAV requires helper virus function, e.g., adenovirus, herpes virus, or vaccinia, in order for a productive infection to occur.

AAV has a wide host range and is able to replicate in cells from any species in the presence of a suitable helper virus. AAV has not been associated with any human or animal disease and does not appear to adversely affect the biological properties of the host cell upon integration.

AAV vectors can be engineered to carry a heterologous nucleic acid sequence of interest (e.g., a selected gene encoding a therapeutic protein, a nucleic acid such as an antisense molecule, a ribozyme, a miRNA, etc.) by deleting, in whole or in part, the internal portion of the AAV genome and inserting the heterologous nucleic acid sequence of interest between the inverted terminal repeats (ITRs). The ITRs remain functional in such vectors allowing replication and packaging of the recombinant adeno-associated virus (rAAV) containing the heterologous nucleic acid sequence of interest. The heterologous nucleic acid sequence is also typically linked to a promoter sequence capable of driving expression of the nucleic acid in the patient's target cells. Termination signals, such as polyadenylation sites, can also be included in the vector. The rAAV genome DNA is packaged in a viral capsid, a protein shell containing a mixture of three capsid proteins (VP1, VP2 and VP3) arranged in icosahedral symmetry.

Recombinant adeno-associated virus (rAAV) have shown excellent therapeutic promise in several early phase clinical trials by multiple groups. Development of this new class of biologic product towards approval will involve improvements in vector characterization and quality control methods, including a better understanding of how vector design and manufacturing process parameters affect impurity profiles in clinical grade vectors.

One of the challenges associated with the production of rAAV is the formation of “non-full” rAAV particles that do not contain complete genetic material. Non-full rAAV particles, as used herein, refer to a range of particles, or variants, including “empty” particles and “partial” particles. “Partial” particles herein refers to rAAV particles with some genetic material, but not complete genetic material as in full particles. Questions about the impact of non-full particles (including empty and partial particles) on the clinical safety and effectiveness of rAAV-mediated gene expression have necessitated the development of purification processes to remove or separate these species from the full particles. The development of a robust and scalable purification process to efficiently separate non-full and full AAV particles remains a challenge, given the structural similarity between these types of rAAV particles. The rAAV particles differ by the presence and the length of single stranded DNA genome in the rAAV. Different techniques have been developed to separate full rAAV particles from non-full particles. However, these techniques often afford the designed full rAAV particles with low purity and/or low yield.

Thus, there remains a need for the development of new systems and methods for purification of full rAAV particles from non-full particles, including purification from empty or partial particles, with high purity and/or high yield.

### BRIEF SUMMARY OF THE INVENTION

This application relates to methods and systems for the purification of full recombinant adeno-associated virus (rAAV) particles from an rAAV preparation comprising full rAAV particles and non-full particles, which may include empty and/or partial particles, using column chromatography techniques.

In one general aspect, the application relates to a method for purifying full recombinant adeno-associated virus (rAAV) particles, the method comprising:
(a). providing an rAAV preparation comprising the full rAAV particles and non-full particles;
(b). loading the rAAV preparation in a loading buffer to a column comprising a chromatography medium, wherein the full rAAV particles have a higher binding affinity to the chromatography medium than the non-full particles; and
(c). eluting the full rAAV particles bound to the chromatography medium with an elution buffer to obtain a purified preparation.

In some embodiments, the non-full particles comprise empty particles.

In some embodiments, the non-full particles comprise partial particles.

In some embodiments, the non-full particles comprise both empty particles and partial particles.

In some embodiments, the non-full particles do not bind to the chromatography medium and flow through the column.

In some embodiments, the partial particles do not bind to the chromatography medium and flow through the column.

In some embodiments, the quantity of the full and empty rAAV particles applied to the column exceeds the binding capacity of the chromatography medium, such that the empty particles bound to the chromatography medium are displaced by full rAAV particles into a load flowthrough from the column.

In some embodiments, the quantity of the full and non-full rAAV particles applied to the column exceeds the binding capacity of the chromatography medium, such that the empty particles bound to the chromatography medium are displaced by partial and full rAAV particles into a load flowthrough from the column.

In some embodiments, the quantity of the full and non-full rAAV particles applied to the column exceeds the binding capacity of the chromatography medium, such that the empty and partial particles bound to the chromatography medium are displaced by the full rAAV particles into a load flowthrough from the column.

In some embodiments, the chromatography medium is an ion exchange column chromatography medium, preferably an anion exchange chromatography medium.

In some embodiments, the column chromatography medium is selected from the group consisting of Poros 50 HQ, Poros 50 D, Poros 50 PI, Capto ImpRes Q, and Poros XQ, preferably Poros XQ.

In some embodiments, the column chromatography medium is a monolith such as CIMmultus™ QA Monolithic Column.

In some embodiments, the loading buffer comprises at least one buffer selected from the group consisting of Tris, Bis-tris, Bis-tris propane, Tris acetate, ethanolamine and phosphate.

In some embodiments, the loading buffer comprises at least one salt of a cation selected from the group consisting of K(I), Li(I), Ca(II), Mg(II), Cu(II), Ba(II)), Co(II), Ni(II), Mn(II), Zn(II), Cd(II), Pb(II), Fe(III), Fe(II), Na(I), and NH₄⁺. The anionic component of the salt is not determinative.

In some embodiments, the loading buffer has a pH of about 6-10, preferably 8-9.

In some embodiments, the elution buffer comprises at least one buffer selected from the group consisting of Tris, Bis-tris, Bis-tris propane, Tris acetate, ethanolamine and phosphate.

In some embodiments, the elution buffer comprises at least one salt of a cation selected from the group consisting of K(I), Li(I), Ca(II), Mg(II), Cu(II), Ba(II)), Co(II), Ni(II), Mn(II), Zn(II), Cd(II), Pb(II), Fe(III), Fe(II), Na(I), and NH₄⁺. The anionic component of the salt is not determinative.

In some embodiments, the elution buffer has a pH of about 6-10, preferably 8-9.

In some embodiments, the yield of the purified full rAAV particles is no less than 70%, preferably no less than 80%, more preferably no less than 90%, and most preferably no less than 95%.

In some embodiments, the purified preparation is substantially free of the non-full particles. In other embodiments, the purified preparation comprises an increased ratio of the full rAAV particles to the non-full particles than that of the rAAV preparation. Preferably, the ratio of the full rAAV particles to the non-full particles in the purified preparation is no less than 9:1, such as no less than 10:1, 15:1, 20:1, 25:1, 30:1, 35:1, 40:1, 45:1 or 50:1, or any ratio in between, more preferably no less than 49:1.

In some embodiments, the full rAAV particles comprise a transgene that encodes a polypeptide, or a nucleic acid selected from the group consisting of a siRNA, an antisense molecule, a miRNA, a ribozyme and a shRNA.

In some embodiments, the rAAV particles comprise a capsid derived from an AAV serotype.

In another embodiment, the rAAV particles comprise a capsid derived from an engineered capsid capable of binding an ion exchange chromatography medium.

In another general aspect, the application relates to a method for purifying full recombinant adeno-associated virus (rAAV) particles, the method comprising:
(a). providing an rAAV preparation comprising full rAAV particles and non-full particles;
(b). loading a first batch of the rAAV preparation in a loading buffer to a first column comprising a first chromatography medium, wherein the full rAAV particles have a higher binding affinity to the first chromatography medium than the non-full particles, and the quantity of the full rAAV particles and non-full particles applied to the first column exceeds the binding capacity of the first chromatography medium such that the non-full particles bound to the first chromatography medium are displaced by the full rAAV particles into a first load flowthrough from the first column;
(c). loading the first load flowthrough to a second column comprising a second chromatography medium to obtain a partially loaded (i.e. not fully saturated) second column, preferably, the second chromatography medium is the same type as the first chromatography medium;
(d). optionally, washing the first column with a wash buffer to obtain a washed first column;
(e). bypassing the second column after the step (c) or after the wash step (d) if the wash step (d) is performed, and eluting the full rAAV particles bound to the first chromatography medium with an elution buffer to obtain a first eluate from the first column and an eluted first column, wherein the first eluate comprises an increased ratio of the full rAAV particles to the non-full rAAV particles;
(f). loading a second batch of the rAAV preparation in the loading buffer to the partially loaded second column, wherein the quantity of the full rAAV particles and non-full particles applied to the second column exceeds the binding capacity of the second chromatography medium such that the non-full particles bound to the second chromatography medium are displaced by full rAAV particles into a second load flowthrough from the second column;
(g). loading the second load flowthrough to the eluted first column to obtain a partially loaded first column;
(h). optionally, washing the second column with a wash buffer to obtain a washed second column;
(i). bypassing the first column after step (g) or after the wash step (h) if the wash step (h) is performed, and eluting the full rAAV particles bound to the second chromatography medium with an elution buffer to obtain a second eluate and an eluted second column, wherein the second eluate comprises an increased ratio of the full rAAV particles to the non-full rAAV particles; and
(j). combining the first eluate and second eluate to produce a purified preparation.

In some embodiments, the non-full particles comprise empty particles.

In some embodiments, the non-full particles comprise partial particles.

In some embodiments, the non-full particles comprise both empty particles and partial particles.

In some embodiments, when the non-full particles comprise both empty and partial particles, the second eluate in step (i) comprises an increased ratio of full and partial rAAV particles to the empty rAAV particles.

In some embodiments, more than two columns can be used, and two is the minimum. In a three column set-up, full particles are enriched in the first column while partial particles, when present, are enriched in the second column, and empty particles are enriched in the third column. In some embodiments, where impurities or aggregates are present in the preparation, the impurities or aggregates may bind the first column with greater affinity than the full particles. In these embodiments, the full particles will enrich on the subsequent column(s).

In some embodiments, the first chromatography medium and/or the second chromatography medium is an ion exchange column chromatography medium, preferably an anion exchange chromatography medium.

In some embodiments, the second column is partially loaded after loading the first flowthrough.

In some embodiments, the steps (b) to (i) are performed with one cycle or more cycles.

In some embodiments, the eluted column can be subject to subsequent steps beneficial or necessary to maintain consistent column binding capacity throughout the cycles before the next cycle of loading. For example, such steps include, but are not limited to, stripping the column, cleaning and/or sanitizing the column, and/or re-equilibrating the column.

In some embodiments, the first or second column chromatography medium is selected from the group consisting of Poros 50 HQ, Poros 50 D, Poros 50 PI, Capto ImpRes Q, and Poros XQ, preferably Poros XQ.

In some embodiments, the column chromatography medium is a monolith such as CIMmultus™ QA Monolithic Column.

In some embodiments, the loading buffer comprises at least one buffer selected from the group consisting of Tris, Bis-tris, Bis-tris propane, Tris acetate, ethanolamine, and phosphate.

In some embodiments, the loading buffer comprises at least one salt of a cation selected from the group consisting of K(I), Li(I), Ca(II), Mg(II), Cu(II), Ba(II)), Co(II), Ni(II), Mn(II), Zn(II), Cd(II), Pb(II), Fe(III), Fe(II), Na(I), and NH₄⁺. The anionic component of the salt is not determinative.

In some embodiments, the loading buffer has a pH of about 6-10, preferably 8-9.

In some embodiments, the loading buffer comprises at least one surfactant.

In some embodiments, the surfactant in the loading buffer is selected from the group consisting of poloxamer 188, polysorbate 80, polysorbate 20, NP-40, Triton X-100, and Triton CG-110.

In some embodiments, the concentration of the surfactant in the loading buffer is between 0.0001% to 0.1%.

In some embodiments, the elution buffer comprises at least one buffer selected from the group consisting of Tris, Bis-tris, Bis-tris propane, Tris acetate, ethanolamine and phosphate.

In some embodiments, the elution buffer comprises at least one salt of a cation selected from the group consisting of K(I), Li(I), Ca(II), Mg(II), Cu(II), Ba(II)), Co(II), Ni(II), Mn(II), Zn(II), Cd(II), Pb(II), Fe(III), Fe(II), Na(I), and NH₄⁺. The anionic component of the salt is not determinative.

In some embodiments, the elution buffer has a pH of about 6-10, preferably 8-9.

In some embodiments, the elution buffer comprises at least one surfactant.

In some embodiments, the surfactant in the elution buffer is selected from the group consisting of poloxamer 188, polysorbate 80, polysorbate 20, NP-40, Triton X-100, and Triton CG-110.

In some embodiments, the concentration of the surfactant in the elution buffer is between 0.0001% to 0.1%.

In some embodiments, the yield of the purified full rAAV particles is no less than 70%, preferably no less than 80%, more preferably no less than 90%, and most preferably no less than 95%.

In some embodiments, the purified preparation is substantially free of the non-full particles. In other embodiments, the purified preparation comprises an increased ratio of the full rAAV particles to the non-full particles than that of the rAAV preparation. Preferably, the ratio of the full particles to the non-full particles in the purified preparation is no less than 9:1, such as no less than 10:1, 15:1, 20:1, 25:1, 30:1, 35:1, 40:1, 45:1 or 50:1, or any ratio in between, more preferably no less than 49:1.

In some embodiments, the full rAAV particles comprise a transgene that encodes a polypeptide, nucleic acid that encodes a protein or is transcribed into a transcript of interest, or nucleic acid, selected from the group consisting of a siRNA, an antisense molecule, miRNA a ribozyme and a shRNA.

In some embodiments, the rAAV particles comprise a capsid derived from one or more AAVs selected from the group consisting of AAV1, AAV2, AAV3, AAV4, AAV5, AAV6, AAV7, AAV8, AAV9, AAV9.47, AAV9(hu14), AAV10, AAV11, AAV12, Rh8, Rh10, Rh74, AAV3B, AAV-218, LK03, RHM4-1, DJ, DJ8, NP59, Anc-80 and variants thereof, including the variants of AAV capsids set forth in Pulicherla et al., Mol. Ther., 19(6) 1070-1078 (2011) (describing AAV9 variants, including AAV9.47 among others), U.S. Pat. No. 7,906,111 (describing AAV9(hu14) among others), U.S. Pat. No. 10,532,111 (describing NP59 among others), U.S. Pat. No. 10,738,087 (describing Anc-80 among others), U.S. Pat. No. 9,169,299 (describing “LK03”), U.S. Pat. No. 9,840,719 (describing “RHM4-1”), U.S. Pat. Nos. 7,749,492, 7,588,772 (describing “DJ” and “DJ8”), U.S. Pat. No. 9,587,282, and patent applications WO2012/145601, WO2013/158879, WO2015/013313, WO2018/156654, US2013/0059732, all of which are incorporated herein by reference in their entireties.

In another general aspect, the application relates to a method for purifying full recombinant adeno-associated virus (rAAV) particles comprising:
(a). providing an rAAV preparation comprising the full rAAV particles and non-full particles;
(b). loading the rAAV preparation in a loading buffer to a column comprising a chromatography medium, wherein the loading buffer comprises CaCl₂), and the full rAAV particles bind to the chromatography medium; and
(c). eluting the full rAAV particles bound to the chromatography medium with an elution buffer to obtain a purified preparation, wherein the elution buffer optionally comprises CaCl₂).

In some embodiments, the chromatography medium is an ion exchange column chromatography medium, preferably an anion exchange chromatography medium.

In some embodiments, the column chromatography medium is selected from the group consisting of Poros 50 HQ, Poros 50 D, Poros 50 PI, Capto ImpRes Q, and Poros XQ, preferably Poros XQ.

In some embodiments, the loading buffer comprises at least one surfactant.

In some embodiments, the surfactant in the loading buffer is selected from the group consisting of poloxamer 188, polysorbate 80, polysorbate 20, NP-40, Triton X-100, and Triton CG-110.

In some embodiments, the concentration of the surfactant in the loading buffer is between 0.0001% to 0.1%.

In some embodiments, the loading buffer comprises at least one buffer selected from the group consisting of Tris, Bis-tris, Bis-tris propane, Tris acetate, ethanolamine, and phosphate.

In some embodiments, the elution buffer comprises at least one buffer selected from the group consisting of Tris, Bis-tris, Bis-tris propane, Tris acetate, ethanolamine and phosphate.

In some embodiments, the loading buffer comprises a salt of a cation selected from the group consisting of K(I), Li(I), Ca(II), Mg(II), Cu(II), Ba(II)), Co(II), Ni(II), Mn(II), Zn(II), Cd(II), Pb(II), Fe(III), Fe(II), Na(I), and NH₄⁺.

In some embodiments, the elution buffer comprises a salt of a cation selected from the group consisting of K(I), Li(I), Ca(II), Mg(II), Cu(II), Ba(II)), Co(II), Ni(II), Mn(II), Zn(II), Cd(II), Pb(II), Fe(III), Fe(II), Na(I), and NH₄⁺.

In some embodiments, the loading buffer comprises about 0-10 mM CaCl₂), preferably 0.1-2.5 mM CaCl₂).

In some embodiments, the elution buffer comprises about 0.1-20 mM CaCl₂), preferably 5-10 mM CaCl₂)

In some embodiments, the loading buffer comprises about 0-100 mM LiCl, preferably 0-75 mM LiCl.

In some embodiments, the elution buffer comprises about 0-200 mM LiCl, preferably 0-150 mM LiCl.

In some embodiments, the loading buffer comprises about 0-10 mM CuCl₂, preferably 0.1-3 mM CuCl₂.

In some embodiments, the elution buffer comprises about 0-10 mM CuCl₂, preferably 0-3 mM CuCl₂.

In some embodiments, the loading buffer further comprises NaCl and/or MgCl₂.

In some embodiments, the loading buffer has a pH of about 6-10, preferably 8-9.

In some embodiments, the elution buffer further comprises NaCl and/or MgCl₂.

In some embodiments, the elution buffer comprises at least one surfactant.

In some embodiments, the surfactant in the elution buffer is selected from the group consisting of poloxamer 188, polysorbate 80, polysorbate 20, NP-40, Triton X-100, and Triton CG-110.

In some embodiments, the concentration of the surfactant in the elution buffer is between 0.0001% to 0.1%.

In some embodiments, the elution buffer has a pH of about 6-10, preferably 8-9.

In some embodiments, the yield of the purified full rAAV particles is no less than 70%, preferably no less than 80%, more preferably no less than 90%, and most preferably no less than 95%.

In some embodiments, the purified preparation is substantially free of the non-full particles. In other embodiments, the purified preparation comprises an increased ratio of the full rAAV particles to the non-full particles than that of the rAAV preparation. Preferably, the ratio of the full particles to the non-full particles in the purified preparation is no less than 9:1, such as no less than 10:1, 15:1, 20:1, 25:1, 30:1, 35:1, 40:1, 45:1 or 50:1, or any ratio in between, preferably no less than 49:1.

In some embodiments, the full rAAV particles comprise a transgene that encodes a polypeptide, or a nucleic acid selected from the group consisting of a siRNA, an antisense molecule, miRNA a ribozyme and a shRNA.

In some embodiments, the rAAV particles are derived from an AAV selected from the group consisting of AAV1, AAV2, AAV3, AAV4, AAV5, AAV6, AAV7, AAV8, AAV9, AAV9.47, AAV9(hu14), AAV10, AAV11, AAV12, Rh8, Rh10, Rh74, AAV3B, AAV-218, LK03, RHM4-1, DJ, DJ8, NP59, Anc-80 and variants thereof, including the variants of AAV capsids set forth in Pulicherla et al., Mol. Ther., 19(6) 1070-1078 (2011) (describing AAV9 variants, including AAV9.47 among others), U.S. Pat. No. 7,906,111 (describing AAV9(hu14) among others), U.S. Pat. No. 10,532,111 (describing NP59 among others), U.S. Pat. No. 10,738,087 (describing Anc-80 among others), U.S. Pat. No. 9,169,299 (describing “LK03”), U.S. Pat. No. 9,840,719 (describing “RHM4-1”), U.S. Pat. Nos. 7,749,492, 7,588,772 (describing “DJ” and “DJ8”), U.S. Pat. No. 9,587,282, and patent applications WO2012/145601, WO2013/158879, WO2015/013313, WO2018/156654, US2013/0059732, all of which are incorporated herein by reference in their entireties.

In one general aspect, the application relates to a method for purifying partial rAAV particles, the method comprising:
(a). providing a non-full rAAV preparation comprising empty and partial particles;
(b). loading the non-full rAAV preparation in a loading buffer to a column comprising a chromatography medium, wherein the partial rAAV particles have a higher binding affinity to the chromatography medium than the empty particles; and
(c). eluting the partial rAAV particles bound to the chromatography medium with an elution buffer to obtain a purified preparation.

In some embodiments, the quantity of the partial and empty rAAV particles applied to the column exceeds the binding capacity of the chromatography medium, such that the empty particles bound to the chromatography medium are displaced by partial rAAV particles into a load flowthrough from the column.

In some embodiments, the chromatography medium is an ion exchange column chromatography medium, preferably an anion exchange chromatography medium.

In some embodiments, the column chromatography medium is selected from the group consisting of Poros 50 HQ, Poros 50 D, Poros 50 PI, Capto ImpRes Q, and Poros XQ, preferably Poros XQ.

In some embodiments, the column chromatography medium is a monolith such as CIMmultus™ QA Monolithic Column.

In some embodiments, the loading buffer comprises at least one buffer selected from the group consisting of Tris, Bis-tris, Bis-tris propane, Tris acetate, ethanolamine, and phosphate.

In some embodiments, the loading buffer comprises at least one salt of a cation selected from the group consisting of K(I), Li(I), Ca(II), Mg(II), Cu(II), Ba(II)), Co(II), Ni(II), Mn(II), Zn(II), Cd(II), Pb(II), Fe(III), Fe(II), Na(I), and NH₄⁺. The anionic component of the salt is not determinative.

In some embodiments, the loading buffer has a pH of about 6-10, preferably 8-9.

In some embodiments, the loading buffer comprises at least one surfactant.

In some embodiments, the surfactant in the loading buffer is selected from the group consisting of poloxamer 188, polysorbate 80, polysorbate 20, NP-40, Triton X-100, and Triton CG-110.

In some embodiments, the concentration of the surfactant in the loading buffer is between 0.0001% to 0.1%.

In some embodiments, the elution buffer comprises at least one buffer selected from the group consisting of Tris, Bis-tris, Bis-tris propane, Tris acetate, ethanolamine and phosphate.

In some embodiments, the elution buffer comprises at least one salt of a cation selected from the group consisting of K(I), Li(I), Ca(II), Mg(II), Cu(II), Ba(II)), Co(II), Ni(II), Mn(II), Zn(II), Cd(II), Pb(II), Fe(III), Fe(II), Na(I), and NH₄⁺. The anionic component of the salt is not determinative.

In some embodiments, the elution buffer has a pH of about 6-10, preferably 8-9.

In some embodiments, the elution buffer comprises at least one surfactant.

In some embodiments, the surfactant in the elution buffer is selected from the group consisting of poloxamer 188, polysorbate 80, polysorbate 20, NP-40, Triton X-100, and Triton CG-110.

In some embodiments, the concentration of the surfactant in the elution buffer is between 0.0001% to 0.1%.

In some embodiments, the purified preparation is substantially free of the empty particles. In other embodiments, the purified preparation comprises an increased ratio of the partial rAAV particles to the empty particles than that of the rAAV preparation. Preferably, the ratio of the partial rAAV particles to the empty particles in the purified preparation is no less than 9:1, such as no less than 10:1, 15:1, 20:1, 25:1, 30:1, 35:1, 40:1, 45:1 or 50:1, or any ratio in between, more preferably no less than 49:1.

In some embodiments, the rAAV particles comprise a capsid derived from an AAV serotype.

In another embodiment, the rAAV particles comprise a capsid derived from an engineered capsid capable of binding an ion exchange chromatography medium.

In some embodiments, the rAAV particles are derived from an AAV selected from the group consisting of AAV1, AAV2, AAV3, AAV4, AAV5, AAV6, AAV7, AAV8, AAV9, AAV9.47, AAV9(hu14), AAV10, AAV11, AAV12, Rh8, Rh10, Rh74, AAV3B, AAV-218, LK03, RHM4-1, DJ, DJ8, NP59, Anc-80 and variants thereof, including the variants of AAV capsids set forth in Pulicherla et al., Mol. Ther., 19(6) 1070-1078 (2011) (describing AAV9 variants, including AAV9.47 among others), U.S. Pat. No. 7,906,111 (describing AAV9(hu14) among others), U.S. Pat. No. 10,532,111 (describing NP59 among others), U.S. Pat. No. 10,738,087 (describing Anc-80 among others), U.S. Pat. No. 9,169,299 (describing “LK03”), U.S. Pat. No. 9,840,719 (describing “RHM4-1”), U.S. Pat. Nos. 7,749,492, 7,588,772 (describing “DJ” and “DJ8”), U.S. Pat. No. 9,587,282, and patent applications WO2012/145601, WO2013/158879, WO2015/013313, WO2018/156654, US2013/0059732, all of which are incorporated herein by reference in their entireties.

In one general aspect, the application relates to a method for purifying empty rAAV particles, the method comprising:
(a). providing an rAAV preparation comprising the empty rAAV particles, and at least one of full and partial rAAV particles;
(b). loading the rAAV preparation in a loading buffer to a column comprising a chromatography medium, wherein the empty rAAV particles have a higher binding affinity to the chromatography medium than the full or partial particles, and the quantity of the empty rAAV particles and the at least one of full and partial particles applied to the column exceeds the binding capacity of the chromatography medium, such that the at least one of full and partial particles bound to the chromatography medium are displaced by the empty rAAV particles into the flowthrough from the column; and
(c). eluting the empty rAAV particles bound to the chromatography medium with an elution buffer to obtain a purified preparation.

In some embodiments, the quantity of the empty and the at least one of the full and partial particles applied to the column exceeds the binding capacity of the chromatography medium, such that the at least one of full and partial particles bound to the chromatography medium are displaced by the empty rAAV particles into a load flowthrough from the column.

In some embodiments, the rAAV preparation comprises full particles.

In some embodiments, the rAAV preparation comprises partial particles.

In some embodiments, the rAAV preparation comprises both full and partial particles.

In some embodiments, the chromatography medium is an ion exchange column chromatography medium, preferably an anion exchange chromatography medium.

In some embodiments, the column chromatography medium is selected from the group consisting of Poros 50 HQ, Poros 50 D, Poros 50 PI, Capto ImpRes Q, and Poros XQ, preferably Poros XQ.

In some embodiments, the column chromatography medium is a monolith such as CIMmultus™ QA Monolithic Column.

In some embodiments, the loading buffer comprises at least one buffer selected from the group consisting of Tris, Bis-tris, Bis-tris propane, Tris acetate, ethanolamine, and phosphate.

In some embodiments, the loading buffer comprises at least one salt of a cation selected from the group consisting of K(I), Li(I), Ca(II), Mg(II), Cu(II), Ba(II)), Co(II), Ni(II), Mn(II), Zn(II), Cd(II), Pb(II), Fe(III), Fe(II), Na(I), and NH₄⁺. The anionic component of the salt is not determinative.

In some embodiments, the loading buffer has a pH of about 6-10, preferably 8-9.

In some embodiments, the loading buffer comprises at least one surfactant.

In some embodiments, the surfactant in the loading buffer is selected from the group consisting of poloxamer 188, polysorbate 80, polysorbate 20, NP-40, Triton X-100, and Triton CG-110.

In some embodiments, the concentration of the surfactant in the loading buffer is between 0.0001% to 0.1%.

In some embodiments, the elution buffer comprises at least one buffer selected from the group consisting of Tris, Bis-tris, Bis-tris propane, Tris acetate, ethanolamine and phosphate.

In some embodiments, the elution buffer comprises at least one salt of a cation selected from the group consisting of K(I), Li(I), Ca(II), Mg(II), Cu(II), Ba(II)), Co(II), Ni(II), Mn(II), Zn(II), Cd(II), Pb(II), Fe(III), Fe(II), Na(I), and NH₄⁺. The anionic component of the salt is not determinative.

In some embodiments, the elution buffer has a pH of about 6-10, preferably 8-9.

In some embodiments, the elution buffer comprises at least one surfactant.

In some embodiments, the surfactant in the elution buffer is selected from the group consisting of poloxamer 188, polysorbate 80, polysorbate 20, NP-40, Triton X-100, and Triton CG-110.

In some embodiments, the concentration of the surfactant in the elution buffer is between 0.0001% to 0.1%.

In some embodiments, the purified preparation is substantially free of the empty particles. In other embodiments, the purified preparation comprises an increased ratio of the partial rAAV particles to the empty particles than that of the rAAV preparation. Preferably, the ratio of the partial rAAV particles to the empty particles in the purified preparation is no less than 3:1, such as no less than 3.5:1, 4:1, 4.5:1, 5:1, 5.5:1, 6:1, 6.5:1, 7:1 or 7.5:1, or any ratio in between, more preferably no less than 4:1.

In some embodiments, the rAAV particles comprise a capsid derived from an AAV serotype.

In another embodiment, the rAAV particles comprise a capsid derived from an engineered capsid capable of binding an ion exchange chromatography medium.

In some embodiments, the rAAV particles are derived from an AAV selected from the group consisting of AAV1, AAV2, AAV3, AAV4, AAV5, AAV6, AAV7, AAV8, AAV9, AAV9.47, AAV9(hu14), AAV10, AAV11, AAV12, Rh8, Rh10, Rh74, AAV3B, AAV-218, LK03, RHM4-1, DJ, DJ8, NP59, Anc-80 and variants thereof, including the variants of AAV capsids set forth in Pulicherla et al., Mol. Ther., 19(6) 1070-1078 (2011) (describing AAV9 variants, including AAV9.47 among others), U.S. Pat. No. 7,906,111 (describing AAV9(hu14) among others), U.S. Pat. No. 10,532,111 (describing NP59 among others), U.S. Pat. No. 10,738,087 (describing Anc-80 among others), U.S. Pat. No. 9,169,299 (describing “LK03”), U.S. Pat. No. 9,840,719 (describing “RHM4-1”), U.S. Pat. Nos. 7,749,492, 7,588,772 (describing “DJ” and “DJ8”), U.S. Pat. No. 9,587,282, and patent applications WO2012/145601, WO2013/158879, WO2015/013313, WO2018/156654, US2013/0059732, all of which are incorporated herein by reference in their entireties.

The details of one or more embodiments of the invention are set forth in the description below. Other features and advantages will be apparent from the following detailed description, and the appended claims.

### BRIEF DESCRIPTION OF THE SEVERAL VIEWS OF THE DRAWINGS

The foregoing summary, as well as the following detailed description of the invention, will be better understood when read in conjunction with the appended drawings. It should be understood that the invention is not limited to the precise embodiments shown in the drawings.

FIGS. 1A-C demonstrate the separation of full rAAV particles and non-full particles on Poros 50 HQ resin at three different pH values: pH 8.0 (FIG. 1A), pH 8.6 (FIG. 1B), and pH 9.25 (FIG. 1C).

FIGS. 2A-D demonstrate the separation of full rAAV particles and non-full particles on Poros 50 D resin at different pH values with different buffers: pH 8.6 and 50 mM Tris (FIG. 2A), pH 8.6 and 25 mM Tris (FIG. 2B), pH 8.0 and 25 mM Tris (FIG. 2C), and pH 9.25 and 25 mM Tris (FIG. 1D).

FIGS. 3A-E demonstrate the separation of full rAAV particles and non-full particles on Poros 50 PI resin at different pH values with different binding strengths: pH 8.0 without NaCl (FIG. 3A), pH 8.6 without NaCl (FIG. 3B), pH 8.6 and 30 mM NaCl (FIG. 3C), pH 9.2 without NaCl (FIG. 4C), and pH 9.2 and 30 mM NaCl (FIG. 3E).

FIGS. 4A-C demonstrate the separation of full rAAV particles and non-full particles on Capto ImpRes Q resin at different pH values with different buffers: pH 8.0 and 50 mM Tris without NaCl (FIG. 4A), pH 8.6 and 25 mM Tris without NaCl (FIG. 4B), and pH 9.0 and 25 mM Tris without NaCl (FIG. 4C).

FIG. 5 demonstrates that the separation of full rAAV particles and non-full particles on Poros XQ resin was best at pH 8.75 compared to pH 8 and pH 9.25.

FIG. 6 demonstrates the effect of binding salt (1.6, 5, and 6.8 mS/cm) on the separation of full rAAV particles and non-full particles on Poros XQ resin.

FIG. 7 demonstrates the effect of flow rate on the separation of full rAAV particles and non-full particles on Poros XQ resin.

FIG. 8A demonstrate the HPLC analysis of a sample with low load, and FIG. 8B demonstrates the HPLC analysis of the sample with breakthrough load.

FIGS. 9A-D demonstrate the effect of binding strengths with salt under the breakthrough condition: 45 mM NaCl (FIG. 9A), 60 mM NaCl (FIG. 9B), 75 mM NaCl (FIG. 9C), and 90 mM NaCl (FIG. 9D).

FIGS. 10A-D demonstrate the step elution for different binding strengths with salt: 60 mM NaCl (FIG. 10A), 75 mM NaCl (FIG. 10B), 90 mM NaCl (FIG. 10C), and 120 mM NaCl (FIG. 10D).

FIGS. 11A-B demonstrate a sample loaded onto PXQ resin in 50 mM Tris pH 8.5 buffer with 60 mM NaCl. Elution was done with 300 mM NaCl 50 mM Tris pH 8.5 buffer using linear gradient. More sample was loaded onto the column in FIG. 11B than in FIG. 11A. These figures demonstrate that displacement chromatography separates full rAAV particles from non-full particles. FIG. 11B further demonstrates that displacement was more pronounced when more sample was loaded, increasing the product purity to nearly 100% with >90% yield.

FIG. 12 demonstrates a sample loaded onto PXQ resin in 50 mM Tris pH 8.5 buffer with 10 mM MgCl₂. Elution was done with 300 mM NaCl 50 mM Tris pH 8.5 buffer. FIG. 12 demonstrates the use of MgCl₂ in load sample at a high concentration of 10 mM removes non-full particles during loading.

FIG. 13 demonstrates a sample loaded onto PXQ resin in 50 mM Tris pH 8.5 buffer with 2.5 mM CaCl₂). Elution was done with 300 mM NaCl 50 mM Tris pH 8.5 buffer. FIG. 13 demonstrates that the addition of CaCl₂) in the load removes non-full particles during loading. Compared to the process in FIG. 12, this process is more robust as the conductivity difference between full particles and non-full particles is greater (>4 mS/cm).

FIGS. 14A-B demonstrate a sample loaded onto PXQ resin in 50 mM Tris pH 8.5 buffer with 1 to 1.5 mM CaCl₂)+2.5 mM MgCl₂+20 mM NaCl. Elution was done with 10 mM CaCl₂, 2.5 mM MgCl₂ 20 mM NaCl 50 mM Tris pH 8.5 buffer. These figures demonstrate the robustness for CaCl₂) concentration. FIG. 14A is with 1.5 mM CaCl₂) in load while FIG. 14B is with 1 mM CaCl₂). The process produced 100% removal of non-full particles without any full particles loss even with 50% changes in CaCl₂) concentration and in presence of other additives. Further upon elution with a CaCl₂) gradient, the full particles do not elute until 5 mM of CaCl₂) is applied, demonstrating a robust difference of 4 mM CaCl₂) between the non-full and full collection. FIG. 14C-D demonstrates the same conditions as FIG. 14B, but with Analytical Ultra Centrifugation (AUC) analysis for drug substance shown in FIG. 14D.

FIG. 15 demonstrates a chromatogram for a sample loaded onto PXQ resin in 50 mM Tris pH 8.5. The column was washed with 50 mM Tris pH 8.5 buffer with 1 mM CaCl₂)+2.5 mM MgCl2. This figure demonstrates that both yield and purity were compromised in the purification of full particles from the non-full particles if CaCl₂) was not added in the sample load. In addition, it was difficult to remove all non-full particles bound to the column via wash. Furthermore, in the subsequent elution with increased CaCl₂) concentration, the bound non-full particles were not stable on the resin and generated a new impurity which in turn compromised the final purity.

FIG. 16 demonstrates the implementation scheme of cyclic displacement chromatography for the separation of full rAAV particles from non-full particles.

FIG. 17 demonstrates a chromatogram for a sample loaded onto PXQ resin in 50 mM Tris pH 8.5 buffer with 58 mM LiCl. Column was eluted with 50 mM Tris pH 8.5+120 mM LiCl buffer. The conductivity difference in the non-full and full elution is 0.2 mS/cm at this condition.

FIG. 18 demonstrates the implementation scheme of cyclic displacement chromatography for the separation of full rAAV particles from non-full particles with additives in at least the loading buffer, and preferably in the wash and elution buffers.

FIGS. 19A-B demonstrate removal of non-full particles during sample loading and recovery of full particles with decreasing pH with gradient or step elution. Loading sample buffer is 50 mM Tris, 2 mM MgCl₂, 2 mM CaCl₂), 20 mM NaCl at pH 8.5. Elution with decreasing pH gradient with 20 mM BisTris-30 mM Acetate, 2 mM MgCl₂, 2 mM CaCl₂) pH 6.0 is shown in FIG. 19A. Elution with pH step with 20 mM BisTris-30 mM Acetate, 2 mM MgCl₂, 2 mM CaCl₂) pH 7.0 and pH 6.0 is shown in FIG. 19B.

FIGS. 20A-B demonstrate that the addition of (NH₄)₂SO₄ into the sample load leads to the flowthrough of non-full particles during loading (FIG. 20B). Sample was loaded onto PXQ resin in 100 mM Tris pH 8.5 buffer with 0.0002% poloxamer 188, 20 mM NaCl, 20 mM (NH₄)₂SO₄ and 2.0 mM MgCl₂. The Elution buffer is a 50 mM Tris pH 8.5 buffer with 0.0002% poloxamer 188 and 300 mM NaCl. In the absence of (NH₄)₂SO₄ (shown in the inset figure) non-full particles were not removed during sample loading at the same buffer conductivity of 7 mS/cm (FIG. 20A).

FIG. 21 demonstrates that the addition of (NH₄)₂SO₄ into the sample load leads to the flow through of non-full particles during sample loading for the monolith column. Sample was loaded onto BIA 1.3 μm monolith column in 50 mM Tris pH 8.5 buffer with 0.0002% poloxamer 188, 20 mM NaCl, 20 mM (NH₄)₂SO₄ and 2.0 mM MgCl₂. The Elution buffer is a 50 mM Tris pH 8.5 buffer with 0.0002% poloxamer 188 and 200 mM NaCl

FIGS. 22A-B demonstrates that the addition of CuCl₂ into the sample load leads to the flow through of non-full particles during sample loading. Sample was loaded onto PXQ resin in 50 mM Tris pH 8.5 buffer with 0.0002% poloxamer 188, 20 mM NaCl, 15 mM (NH₄)₂SO₄, 2.0 mM MgCl₂ and 1.5 mM CuCl₂. The elution buffer is a 180 mM sodium phosphate pH 7.2 buffer with 0.0002% poloxamer 188 and 2 mM MgCl₂.

FIGS. 22 C-E demonstrate that addition of CuCl₂ into the sample load results in increased resolution between the non-full (left most peak) and the full (second to left peak) particles at the analytical scale. In addition, the addition of CuCl₂ lead to a significant reduction in some product variants (peaks following the second peak). This demonstrates that the addition of CuCl₂ in the load removes non-full particles during loading. FIG. 22F shows that Cu(II) leads to the highest increase in the resolution between non-full and full peaks among other ions. The samples are in 50 mM Tris pH 8.5, with 0.5-2 mM of the specified ions.

FIGS. 23A-E demonstrates that with the addition of CuCl₂ non-full particles can be removed during loading and some resolution can be achieved between partial and full particles. The rAAV preparations were loaded onto PXQ resin in 50 mM Tris pH 8.5 buffer with 0.0002% poloxamer 188, 20 mM NaCl, 15 mM NH₄SO₄, 2.0 mM MgCl₂ and 1.5 mM CuCl₂. Bound particles were eluted in stepwise manner with 50 mM sodium acetate pH 6.0 buffer with 0.0002% poloxamer 188 and two different amounts of NaCl. Elution peak 1 is obtained with 50 mM NaCl and elution peak 2 is obtained with 200 mM NaCl (FIGS. 23A-B). FIGS. 23C-E show the sedimentation coefficient distributions of the eluate peaks using analytical ultracentrifugation. As shown in the Table 1 below, peak 1 contains both partial (40.1%) and full particles (49.3%) while peak 2 is primarily enriched in full particles (69.8%) with a lower percentage of partial particles (15.5%) and removal of non-full particles (15.5%).

TABLE 1
Sedimentation Coefficient Distributions

| | Empty | Partial | Full | Full/ |
|---|---|---|---|---|
| | Capsid % | Capsid % | Capsid % | Partial |
| Load | 46.7 | 16 | 30.4 | 1.90 |
| Peak 1 | 4.6 | 40.1 | 49.3 | 1.23 |
| Peak 2 | 15.5 | 8.2 | 69.8 | 8.51 |

FIGS. 24A-G demonstrate the use of three-column displacement chromatography method for the enrichment of rAAV particle variants. The rAAV preparations were loaded onto three PXQ columns that were connected in series. The loading buffer is 50 mM Tris pH 8.5, 75 mM NaCl and 2 mM MgCl₂. The three columns were eluted sequentially using 200 mM NaCl 50 mM Tris buffer pH 8.5 with 0.0002% poloxamer 188 and 2 mM MgCl₂ (FIGS. 24 A-B for the preparative chromatogram). The collected fractions were analyzed using UPLC on IEX column. As shown in FIGS. 24C-D, empty particles flowed through the column, and the majority of empty particles were removed during loading. In addition, analytical IEX chromatograms show that the first column is enriched in the strongest binding particle (highest retention time; FIG. 24E) while the second and third columns are enriched in the second strongest binding and the weakest binding particles, respectively (FIGS. 24F-G). First and second column elution has enriched full particles. This demonstrates the use of displacement chromatography for the enrichment of different particle variants on a three-column setup.

FIGS. 25A-B demonstrate the use of a two-column displacement chromatography method for the enrichment of full particles. The rAAV preparations were loaded onto two PXQ columns connected in series. The loading buffer is 50 mM Tris pH 8.5, 10 mM NaCl, 2.5 mM MgCl₂ and 1 mM CaCl₂). The elution was carried out sequentially from column 1 and column 2 with 50 mM Tris pH 8.5, 200 mM NaCl, 2.5 mM MgCl₂ and 0.0002% poloxamer 188 (FIGS. 25A-B for the preparative chromatogram). The collected fractions were analyzed using UPLC. As shown in FIG. 25C, empty particles flowed through during loading. Impurities in the rAAV preparation bound to the PXQ resin with higher affinity than the full particles. Consequently, these impurities were reduced in the preparation loaded onto column 2, and the full particles were the species having the greatest affinity for binding column 2. Upon elution, the column 2 eluate is more enriched in full particles. The column 2 elution peak thus illustrates better enrichment of full particles than column 1 as shown in FIG. 25D (UPLC analysis).

FIGS. 26A-D demonstrate the use of a three-column displacement chromatography method for the enrichment of partial and full particles. The rAAV preparations were loaded onto three PXQ columns connected in series. The loading buffer is 50 mM Tris pH 8.5, 20 mM NaCl, 15 mM Ammonium Sulfate, 2 mM MgCl₂ and 1.5 mM CuCl₂. The elution was carried out sequentially from column 1, 2 and 3 with 50 mM Tris pH 8.5, 200 mM NaCl, 2.5 mM MgCl₂ and 0.0002% poloxamer 188 (FIGS. 26A-B for the preparative chromatogram). FIGS. 26 C-D show the characterization of the flow through and the eluates by analytical ion exchange chromatography and analytical ultra centrifugation (AUC). Empty particles flowed through during loading (FIG. 26C). The primary eluate peak from column 1 is enriched in full particles and the primary eluate peak from column 3 is enriched in partial particles while column 2 contains both partial and full particles (FIG. 26D).

### DETAILED DESCRIPTION OF THE INVENTION

Various publications, articles and patents are cited or described in the background and throughout the specification: each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein. It must be noted that as used herein and in the appended claims, the singular forms “a,” “an,” and “the” include plural reference unless the context clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the invention can be replaced with the term “consisting of” or “consisting essentially of” to vary scopes of the disclosure.

As used herein, the term “about,” when used in conjunction with a number, refers to any number within ±10%, e.g. ±5%, or ±1%, of the referenced number. For example, a pH of about 5.0 means any pH from 4.5-5.5, inclusive.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The term “vector” refers to any small carrier of nucleic acid molecule, such as a plasmid, phage, transposon, cosmid, chromosome, virus, virion, or other vehicle that can be manipulated by insertion or incorporation of a nucleic acid. Vectors can be used for genetic manipulation (i.e., “cloning vectors”), to introduce/transfer polynucleotides into cells, and to transcribe or translate the inserted polynucleotide in cells. An “expression vector” is a vector that contains a gene or nucleic acid sequence with the necessary regulatory regions needed for expression in a host cell. A vector nucleic acid sequence generally contains at least an origin of replication for propagation in a cell and optionally additional elements, such as a heterologous nucleic acid sequence, expression control element (e.g., a promoter, enhancer), intron, inverted terminal repeats (ITRs), optional selectable marker, polyadenylation signal.

The term “adeno-associated virus (AAV) vector” or “AAV vector” refers to a vector derived from an adeno-associated virus serotype, including without limitation, AAV serotypes such as AAV1, AAV2, AAV3, AAV4, AAV5, AAV6, AAV7, AAV8, AAV9, AAV9.47, AAV9(hu14), AAV10, AAV11, AAV12, Rh8, Rh10, Rh74, AAV3B, AAV-218, LK03, RHM4-1, DJ, DJ8, NP59, Anc-80 and variants thereof, including the variants of AAV capsids set forth in Pulicherla et al., Mol. Ther., 19(6) 1070-1078 (2011) (describing AAV9 variants, including AAV9.47 among others), U.S. Pat. No. 7,906,111 (describing AAV9(hu14) among others), U.S. Pat. No. 10,532,111 (describing NP59 among others), U.S. Pat. No. 10,738,087 (describing Anc-80 among others), U.S. Pat. No. 9,169,299 (describing “LK03”), U.S. Pat. No. 9,840,719 (describing “RHM4-1”), U.S. Pat. Nos. 7,749,492, 7,588,772 (describing “DJ” and “DJ8”), U.S. Pat. No. 9,587,282, and patent applications WO2012/145601, WO2013/158879, WO2015/013313, WO2018/156654, US2013/0059732, all of which are incorporated herein by reference in their entireties. In addition, AAV vector includes AAV that have been engineered for tissue tropism, stability, and transduction efficiency. AAV vectors can have one or more of the AAV wild-type genes deleted in whole or part, preferably replication (rep) and capsid (cap) genes, but retain functional flanking ITR sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging.

The term “AAV virion” refers to a virus particle, such as a wild-type (wt) AAV virus particle, which comprises a linear, single-stranded nucleic acid genome associated with an AAV capsid protein coat.

A recombinant adeno-associated virus (rAAV vector) is derived from adeno-associated virus. AAV vectors are useful as gene therapy vectors as they can introduce nucleic acid/genetic material into cells so that the nucleic acid/genetic material may be maintained in cells. Because AAV are not associated with pathogenic disease in humans, rAAV vectors are able to deliver heterologous nucleic acid sequences (e.g., therapeutic proteins and agents) to human patients without causing substantial AAV pathogenesis or disease.

The term “recombinant,” as a modifier of vector, such as recombinant adeno-associated virus (rAAV) vectors, as well as a modifier of sequences such as recombinant polynucleotides and polypeptides, means that the compositions have been manipulated (i.e., engineered) in a fashion that generally does not occur in nature. An example of a rAAV vector can be where a nucleic acid that is not normally present in the wild-type AAV genome is inserted within the viral genome. For example, a nucleic acid (e.g., gene) encoding a therapeutic protein or polynucleotide sequence is cloned into a vector, with or without 5′, 3′ and/or intron regions that the gene is normally associated within the AAV genome. Although the term “recombinant” is not always used herein in reference to AAV vectors, as well as sequences such as polynucleotides, recombinant forms including AAV vectors, polynucleotides, etc., are expressly included in spite of any such omission.

An rAAV vector is derived from the wild type genome of a virus, such as AAV, by using molecular methods to remove the wild type genome from AAV genome, and replacing with a non-native (heterologous) nucleic acid, such as a nucleic acid encoding a therapeutic protein or nucleic acid molecule of interest. Typically, for AAV, one or both inverted terminal repeat (ITR) sequences of AAV genome are retained in the rAAV vector. An rAAV genome distinguishes from an AAV genome since all or a part of the AAV genome has been replaced with a non-native sequence with respect to the AAV genomic nucleic acid, such as with a heterologous nucleic acid encoding a therapeutic protein or polynucleotide sequence. Incorporation of a non-native sequence therefore defines the AAV as a “recombinant” AAV vector, which can be referred to as an “rAAV vector.” A recombinant AAV vector sequence can be packaged, which is referred to herein as a “particle” for subsequent infection (transduction) of a cell, ex vivo, in vitro or in vivo.

The terms “recombinant AAV virion,” “rAAV virion,” “AAV vector particle,” “full rAAV capsid,” “full rAAV particle”, “full capsid” and “full particle”, as used herein each refer to an infectious, replication-defective virus including an AAV protein shell, encapsulating a nucleic acid molecule comprising a heterologous nucleotide sequence of interest flanked on one or both sides by AAV ITRs. A full rAAV particle is produced in a suitable host cell which has sequences specifying an AAV vector, AAV helper functions and accessory functions introduced therein. In this manner, the host cell is rendered capable of encoding AAV polypeptides that are required for packaging the AAV vector (containing a recombinant nucleotide sequence of interest) into infectious recombinant virion particles for subsequent gene delivery.

The terms “non-full capsid” and “non-full particle,” as used herein, each refer to an AAV particle or virion that includes an AAV particle shell but that lacks a complete nucleic acid molecule comprising the heterologous nucleic acid sequence flanked on one or both sides by AAV ITRs. Such non-full particles do not transfer the complete heterologous nucleic acid sequence into the host cell or cells within an organism. These non-full particles include variants with differing lengths or amounts of incomplete genetic material. The non-full particles that lack enough genetic material, or none at all, to be detected by analytical methods (for example, UPLC and AUC) as having any genetic material are referred to as “empty” particles. The non-full particles that have enough genetic material to be detected by analytical methods as having some genetic material but less than full particles are referred to as “partial” particles. The incomplete genetic material can be intact or fragmented.

Any analytical method known in the art can be used to quantify full and non-full particles, including to determine ratios of full to non-full particles, or non-full to full particles. For example, such methods can be, but are not limited to, Physical Titers Calculation: A260 & A280 Absorbance: analytical anion exchange chromatography (e.g. UPLC); Multi-Angle Light Scattering; Analytical Ultra-Centrifugation (AUC); Cryogenic Electron Microscopy (Cryo-EM); or Charge Detection Mass Spectrometry (CDMS). To exemplify quantitative assessment, several different methods, UPLC, A260 & A280 Absorbance and AUC have been shown in the present disclosure.

A vector “genome” refers to the portion of the recombinant sequence that is ultimately packaged or encapsulated to form an rAAV particle. In cases where recombinant plasmids are used to construct or manufacture recombinant AAV vectors, the AAV vector genome does not include the portion of the “plasmid” that does not correspond to the vector genome sequence of the recombinant plasmid. This non vector genome portion of the recombinant plasmid is referred to as the “plasmid backbone,” which is important for cloning and amplification of the plasmid, a process that is needed for propagation and recombinant virus production, but is not itself packaged or encapsulated into rAAV particles. Thus, a vector “genome” refers to the nucleic acid that is packaged or encapsulated by rAAV.

The term “AAV helper functions” refer to AAV-derived coding sequences (proteins) which can be expressed to provide AAV gene products and AAV vectors that, in turn, function in trans for productive AAV replication and packaging. Thus, AAV helper functions include AAV open reading frames (ORFs), including rep and cap and others such as assembly-activating protein (AAP) for certain AAV serotypes. The Rep expression products have been shown to possess many functions, including, among others: recognition, binding and nicking of the AAV origin of DNA replication; DNA helicase activity; and modulation of transcription from AAV (or other heterologous) promoters. The Cap expression products (capsids) supply necessary packaging functions. AAV helper functions are used to complement AAV functions in trans that are missing from AAV vector genomes.

The term “AAV helper construct” refers generally to a nucleic acid sequence that includes nucleotide sequences providing AAV functions deleted from an AAV vector which is to be used to produce a transducing AVV vector for delivery of a nucleic acid sequence of interest, by way of gene therapy to a subject, for example. AAV helper constructs are commonly used to provide transient expression of AAV rep and/or cap genes to complement missing AAV functions that are necessary for AAV vector replication. Helper constructs generally lack AAV ITRs and can neither replicate nor package themselves. AAV helper constructs can be in the form of a plasmid, phage, transposon, cosmid, virus, or virion. A number of AAV helper constructs have been described, such as plasmids pAAV/Ad and pIM29+45 which encode both Rep and Cap expression products (See, e.g., Samulski et al. (1989) J. Virol. 63:3822-3828; and McCarty et al. (1991) J. Virol. 65:2936-2945). A number of other vectors have been described which encode Rep and/or Cap expression products (See, e.g., U.S. Pat. Nos. 5,139,941 and 6,376,237).

The term “accessory functions” refers to non-AAV derived viral and/or cellular functions upon which AAV is dependent for replication. The term includes proteins and RNAs that are required in AAV replication, including moieties involved in activation of AAV gene transcription, stage specific AAV mRNA splicing, AAV DNA replication, synthesis of Cap expression products and AAV capsid packaging. Viral-based accessory functions can be derived from any of the known helper viruses such as adenovirus, herpesvirus (other than herpes simplex virus type-1) and vaccinia virus.

An “accessory function vector” refers generally to a nucleic acid molecule that includes polynucleotide sequences providing accessory functions. Such sequences can be on an accessory function vector, and transfected into a suitable host cell. The accessory function vector is capable of supporting rAAV virion production in the host cell. Accessory function vectors can be in the form of a plasmid, phage, transposon or cosmid. In addition, the full-complement of adenovirus genes are not required for accessory functions. For example, adenovirus mutants incapable of DNA replication and late gene synthesis have been reported to be permissive for AAV replication (Ito et al., (1970) J. Gen. Virol. 9:243; Ishibashi et al, (1971) Virology 45:317). Similarly, mutants within E2B and E3 regions have been shown to support AAV replication, indicating that the E2B and E3 regions are probably not involved in providing accessory functions (Carter et al., (1983) Virology 126:505). Adenoviruses defective in the El region, or having a deleted E4 region, are unable to support AAV replication. Thus, El A and E4 regions appear necessary for AAV replication, either directly or indirectly (Laughlin et al., (1982) J. Virol. 41:868; Janik et al., (1981) Proc. Natl. Acad. Sci. USA 78: 1925; Carter et al., (1983) Virology 126:505). Other characterized Adenovirus mutants include: EIB (Laughlin et al. (1982), supra; Janik et al. (1981), supra; Ostrove et al., (1980) Virology 104:502); E2A (Handa et al., (1975) J. Gen. Virol. 29:239; Strauss et al., (1976) J. Virol. 17: 140; Myers et al., (1980) J. Virol. 35:665; Jay et al., (1981) Proc. Natl. Acad. Sci. USA 78:2927; Myers et al., (1981) J. Biol. Chem. 256:567); E2B (Carter, Adeno-Associated Virus Helper Functions, in I CRC Handbook of Parvoviruses (P. Tijssen ed., 1990)); E3 (Carter et al. (1983), supra); and E4 (Carter et al. (1983), supra; Carter (1995)). Studies of the accessory functions provided by adenoviruses having mutations in the EIB coding region have produced conflicting results, but ElB55k may be required for AAV virion production, while EIB 19k is not (Samulski et al., (1988) J. Virol. 62:206-210). In addition, International Publication WO 97/17458 and Matshushita et al., (1998) Gene Therapy 5:938-945, describe accessory function vectors encoding various Adenovirus genes. Exemplary accessory function vectors comprise an adenovirus VA RNA coding region, an adenovirus E4 ORF6 coding region, an adenovirus E2A 72 kD coding region, an adenovirus ElA coding region, and an adenovirus EIB region lacking an intact ElB55k coding region. Such accessory function vectors are described, for example, in International Publication No. WO 01/83797.

As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Cross-reactivity differences are usually due to differences in capsid protein sequences/antigenic determinants (e.g., due to VP1, VP2, and/or VP3 sequence differences of AAV serotypes).

Under the traditional definition, a serotype means that the virus of interest has been tested against serum specific for all existing and characterized serotypes for neutralizing activity and no antibodies have been found that neutralize the virus of interest. As more naturally occurring virus isolates are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

rAAV vectors include any viral strain or serotype. As a non-limiting example, a rAAV plasmid or vector genome or particle (capsid) can be based upon any AAV serotype, including, for example, but without limitation, AAV1, AAV2, AAV3, AAV4, AAV5, AAV6, AAV7, AAV8, AAV9, AAV9.47, AAV9(hu14), AAV10, AAV11, AAV12, Rh8, Rh10, Rh74, AAV3B, AAV-218, LK03, RHM4-1, DJ, DJ8, NP59, Anc-80 and variants thereof, including the variants of AAV capsids set forth in Pulicherla et al., Mol. Ther., 19(6) 1070-1078 (2011) (describing AAV9 variants, including AAV9.47 among others), U.S. Pat. No. 7,906,111 (describing AAV9(hu14) among others), U.S. Pat. No. 10,532,111 (describing NP59 among others), U.S. Pat. No. 10,738,087 (describing Anc-80 among others), U.S. Pat. No. 9,169,299 (describing “LK03”), U.S. Pat. No. 9,840,719 (describing “RHM4-1”), U.S. Pat. Nos. 7,749,492, 7,588,772 (describing “DJ” and “DJ8”), U.S. Pat. No. 9,587,282, and patent applications WO2012/145601, WO2013/158879, WO2015/013313, WO2018/156654, US2013/0059732, all of which are incorporated herein by reference in their entireties. The descriptions of all the foregoing AAVs including their sequences information are incorporated by reference in their entireties. Such vectors can be based on the same of strain or serotype (or subgroup or variant), or be different from each other. As a non-limiting example, a rAAV plasmid or vector genome or particle (capsid) based upon one serotype genome can be identical to one or more of the capsid proteins that package the vector. In addition, a rAAV plasmid or vector genome can be based upon an AAV (e.g., AAV2) serotype genome distinct from one or more of the capsid proteins that package the vector genome, in which case at least one of the three capsid proteins could be, but is not limited to, AAV1, AAV2, AAV3, AAV4, AAV5, AAV6, AAV7, AAV8, AAV9, AAV9.47, AAV9(hu14), AAV10, AAV11, AAV12, Rh8, Rh10, Rh74, AAV3B, AAV-218, LK03, RHM4-1, DJ, DJ8, NP59, Anc-80 and variants thereof, including the variants of AAV capsids set forth in Pulicherla et al., Mol. Ther., 19(6) 1070-1078 (2011) (describing AAV9 variants, including AAV9.47 among others), U.S. Pat. No. 7,906,111 (describing AAV9(hu14) among others), U.S. Pat. No. 10,532,111 (describing NP59 among others), U.S. Pat. No. 10,738,087 (describing Anc-80 among others), U.S. Pat. No. 9,169,299 (describing “LK03”), U.S. Pat. No. 9,840,719 (describing “RHM4-1”), U.S. Pat. Nos. 7,749,492, 7,588,772 (describing “DJ” and “DJ8”), U.S. Pat. No. 9,587,282, and patent applications WO2012/145601, WO2013/158879, WO2015/013313, WO2018/156654, US2013/0059732, all of which are incorporated herein by reference in their entireties. rAAV vectors therefore include gene/protein sequences identical to gene/protein sequences characteristic for a particular serotype, as well as mixed serotypes. The various embodiments are applicable to any rAAV or AAV capsid from any source, provided that capsid is capable of binding an ion exchange chromatography column.

In various exemplary embodiments, a rAAV vector includes or consists of a capsid sequence at least 70% or more (e.g., 75%, 80%, 85%, 90%, 95%, 96%, 97%, 98%, 99%, 99.5%, etc.) identical to one or more capsid proteins of AAV1, AAV2, AAV3, AAV4, AAV5, AAV6, AAV7, AAV8, AAV9, AAV9.47, AAV9(hu14), AAV10, AAV11, AAV12, Rh8, Rh10, Rh74, AAV3B, AAV-218, LK03, RHM4-1, DJ, DJ8, NP59, Anc-80 and variants thereof, including the variants of AAV capsids set forth in Pulicherla et al., Mol. Ther., 19(6) 1070-1078 (2011) (describing AAV9) variants, including AAV9.47 among others), U.S. Pat. No. 7,906,111 (describing AAV9(hu14) among others), U.S. Pat. No. 10,532,111 (describing NP59 among others), U.S. Pat. No. 10,738,087 (describing Anc-80 among others), U.S. Pat. No. 9,169,299 (describing “LK03”), U.S. Pat. No. 9,840,719 (describing “RHM4-1”), U.S. Pat. Nos. 7,749,492, 7,588,772 (describing “DJ” and “DJ8”), 9,587,282, and patent applications WO2012/145601, WO2013/158879, WO2015/013313, WO2018/156654, US2013/0059732, all of which are incorporated herein by reference in their entireties. In various exemplary embodiments, a rAAV vector includes or consists of a sequence at least 70% or more (e.g., 75%, 80%, 85%, 90%, 95%, 96%, 97%, 98%, 99%, 99.5%, etc.) identical to one or more ITR(s) of AAV1, AAV2, AAV3, AAV4, AAV5, AAV6, AAV7, AAV8, AAV9, AAV9.47, AAV9(hu14), AAV10, AAV11, AAV12, Rh8, Rh10, Rh74, AAV3B, AAV-218, LK03, RHM4-1, DJ, DJ8, NP59, Anc-80 and variants thereof, including the variants of AAV capsids set forth in Pulicherla et al., Mol. Ther., 19(6) 1070-1078 (2011) (describing AAV9) variants, including AAV9.47 among others), U.S. Pat. No. 7,906,111 (describing AAV9(hu14) among others), U.S. Pat. No. 10,532,111 (describing NP59 among others), U.S. Pat. No. 10,738,087 (describing Anc-80 among others), U.S. Pat. No. 9,169,299 (describing “LK03”), U.S. Pat. No. 9,840,719 (describing “RHM4-1”), U.S. Pat. Nos. 7,749,492, 7,588,772 (describing “DJ” and “DJ8”), U.S. Pat. No. 9,587,282, and patent applications WO2012/145601, WO2013/158879, WO2015/013313, WO2018/156654, US2013/0059732, all of which are incorporated herein by reference in their entireties.

rAAV, including, but not limited to, AAV1, AAV2, AAV3, AAV4, AAV5, AAV6, AAV7, AAV8, AAV9, AAV9.47, AAV9(hu14), AAV10, AAV11, AAV12, Rh8, Rh10, Rh74, AAV3B, AAV-218, LK03, RHM4-1, DJ, DJ8, NP59, Anc-80 and variants thereof, including the variants of AAV capsids set forth in Pulicherla et al., Mol. Ther., 19(6) 1070-1078 (2011) (describing AAV9 variants, including AAV9.47 among others), U.S. Pat. No. 7,906,111 (describing AAV9)(hu14) among others), U.S. Pat. No. 10,532,111 (describing NP59 among others), U.S. Pat. No. 10,738,087 (describing Anc-80 among others), U.S. Pat. No. 9,169,299 (describing “LK03”), U.S. Pat. No. 9,840,719 (describing “RHM4-1”), U.S. Pat. Nos. 7,749,492, 7,588,772 (describing “DJ” and “DJ8”), U.S. Pat. No. 9,587,282, and patent applications WO2012/145601, WO2013/158879, WO2015/013313, WO2018/156654, US2013/0059732, all of which are incorporated herein by reference in their entireties, and variant, hybrid and chimeric sequences, can be constructed using recombinant techniques that are known to the skilled artisan, to include one or more heterologous polynucleotide sequences (transgenes) flanked with one or more functional AAV ITR sequences. Such vectors have one or more of the wild type AAV genes deleted in whole or in part, but retain at least one functional flanking ITR sequence(s), as necessary for the rescue, replication, and packaging of the recombinant vector into a rAAV vector particle. A rAAV vector genome would therefore include sequences required in cis for replication and packaging (e.g., functional ITR sequences)

The terms “nucleic acid” and “polynucleotide” are used interchangeably herein to refer to all forms of nucleic acid, oligonucleotides, including deoxyribonucleic acid (DNA) and ribonucleic acid (RNA). Nucleic acids include genomic DNA, cDNA and antisense DNA, and spliced or unspliced mRNA, IRNA tRNA and inhibitory DNA or RNA (RNAi, e.g., small or short hairpin (sh)RNA, microRNA (miRNA), small or short interfering (si)RNA, trans-splicing RNA, or antisense RNA). Nucleic acids include naturally occurring, synthetic, and intentionally modified or altered polynucleotides. Nucleic acids can be single, double, or triplex, linear or circular, and can be of any length. In discussing nucleic acids, a sequence or structure of a particular polynucleotide may be described herein according to the convention of providing the sequence in the 5′ to 3′ direction.

A “heterologous” nucleic acid sequence refers to a polynucleotide inserted into a AAV plasmid or vector for purposes of vector mediated transfer/delivery of the polynucleotide into a cell. Heterologous nucleic acid sequences are distinct from AAV nucleic acid, i.e., are non-native with respect to AAV nucleic acid. Once transferred/delivered into the cell, a heterologous nucleic acid sequence, contained within the vector, can be expressed (e.g., transcribed, and translated if appropriate). Alternatively, a transferred/delivered heterologous polynucleotide in a cell, contained within the vector, need not be expressed.

The “polypeptides,” “proteins” and “peptides” encoded by the “nucleic acid sequence,” include full-length native sequences, as with naturally occurring proteins, as well as functional subsequences, modified forms or sequence variants so long as the subsequence, modified form or variant retains some degree of functionality of the native full-length protein. Such polypeptides, proteins and peptides encoded by the nucleic acid sequences can be but are not required to be identical to the endogenous protein that is defective, or whose expression is insufficient, or deficient in the treated mammal.

A “transgene” is used herein to conveniently refer to a nucleic acid (e.g., heterologous) that is intended or has been introduced into a cell or organism. Transgenes include any nucleic acid, such as a heterologous nucleic acid encoding a therapeutic protein or polynucleotide sequence.

In a cell having a transgene, the transgene has been introduced/transferred by way of a plasmid or a AAV vector, “transduction” or “transfection” of the cell. The terms “transduce” and “transfect” refer to introduction of a molecule such as a nucleic acid into a host cell (e.g., HEK293) or cells of an organism. The transgene may or may not be integrated into genomic nucleic acid of the recipient cell. If an introduced nucleic acid becomes integrated into the nucleic acid (genomic DNA) of the recipient cell or organism it can be stably maintained in that cell or organism and further passed on to or inherited by progeny cells or organisms of the recipient cell or cells of an organism.

A “host cell” denotes, for example, microorganisms, yeast cells, insect cells, and mammalian cells, that can be, or have been, used as recipients of an AAV vector plasmid, AAV helper construct, an accessory function vector, or other transfer DNA. The term includes the progeny of the original cell which has been transfected. Thus, a “host cell” generally refers to a cell which has been transfected with an exogenous DNA sequence. It is understood that the progeny of a single parental cell may not necessarily be completely identical in morphology or in genomic or total DNA complement as the original parent, due to natural, accidental, or deliberate mutation. Exemplary host cells include human embryonic kidney (HEK) cells such as HEK293.

A “therapeutic protein” as used herein is a peptide or protein that may alleviate or reduce symptoms that result from an insufficient amount, absence or defect in a protein in a cell or subject. A “therapeutic” protein encoded by a transgene can confer a benefit to a subject, e.g., to correct a genetic defect, to correct a gene (expression or functional) deficiency, etc.

Non-limiting examples of heterologous nucleic acids encoding gene products (e.g., therapeutic proteins) which are useful in accordance with the invention include those that may be used in the treatment of a disease or disorder including, but not limited to, “hemostasis” or blood clotting disorders such as hemophilia A, hemophilia A patients with inhibitory antibodies, hemophilia B, deficiencies in coagulation Factors, VII, VIII, IX and X, XI, V, XII, II, von Willebrand factor, combined FV/FVIII deficiency, thalassemia, vitamin K epoxide reductase CI deficiency, gamma-carboxylase deficiency: anemia, bleeding associated with trauma, injury, thrombosis, thrombocytopenia, stroke, coagulopathy, disseminated intravascular coagulation (DIC): over-anticoagulation associated with heparin, low molecular weight heparin, pentasaccharide, warfarin, small molecule antithrombotics (i.e. FXa inhibitors); and platelet disorders such as, Bernard Soulier syndrome, Glanzman thromblastemia, and storage pool deficiency.

In certain embodiments, the disease or disorder affects or originates in the central nervous system (CNS). In certain embodiments, the disease is a neurodegenerative disease. In certain embodiments, the CNS or neurodegenerative disease is Alzheimer's disease, Huntington's disease, ALS, hereditary spastic hemiplegia, primary lateral sclerosis, spinal muscular atrophy, Kennedy's disease, a polyglutamine repeat disease, or Parkinson's disease. In certain embodiments, the CNS or neurodegenerative disease is a polyglutamine repeat disease. In certain embodiments, the polyglutamine repeat disease is a spinocerebellar ataxia (SCA1, SCA2, SCA3, SCA6, SCA7, or SCA17).

In certain embodiments, a heterologous nucleic encodes a protein selected from the group consisting of GAA (acid alpha-glucosidase) for treatment of Pompe disease; ATP7B (copper transporting ATPase2) for treatment of Wilson's disease: alpha galactosidase for treatment of Fabry's disease: ASSI (arginosuccinate synthase) for treatment of citrullinemia Type 1: beta-glucocerebrosidase for treatment of Gaucher disease Type 1: beta-hexosaminidase A for treatment of Tay Sachs disease: SERPINGI (C1 protease inhibitor or C1 esterase inhibitor) for treatment of hereditary angioedema (HAE), also known as C1 inhibitor deficiency type I and type II); and glucose-6-phosphatase for treatment of glycogen storage disease type I (GSDI).

In certain embodiments, a heterologous nucleic acid encodes CFTR (cystic fibrosis transmembrane regulator protein), a blood coagulation (clotting) factor (Factor XIII, Factor IX, Factor VIII, Factor X, Factor VII, Factor VIIa, protein C, etc.) a gain of function blood coagulation factor, an antibody, retinal pigment epithelium-specific 65 kDa protein (RPE65), erythropoietin, LDL receptor, lipoprotein lipase, ornithine transcarbamylase, β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor, insulin-like growth factor 1 or 2, platelet derived growth factor, epidermal growth factor, nerve growth factor, neurotrophic factor −3 and −4, brain-derived neurotrophic factor, glial derived growth factor, transforming growth factor α and β, a cytokine, α-interferon, β-interferon, interferon-γ, interleukin-2, interleukin-4, interleukin 12, granulocyte-macrophage colony stimulating factor, lymphotoxin, a suicide gene product, herpes simplex virus thymidine kinase, cytosine deaminase, diphtheria toxin, cytochrome P450, deoxycytidine kinase, tumor necrosis factor, a drug resistance protein, a tumor suppressor protein (e.g., p53, Rb, Wt-1, NF1. Von Hippel-Lindau (VHL), adenomatous polyposis coli (APC)), a peptide with immunomodulatory properties, a tolerogenic or immunogenic peptide or protein Tregitope or hCDRI, insulin, glucokinase, guanylate cyclase 2D (LCA-GUCY2D), Rab escort protein 1 (choroideremia), LCA 5 (LCA-Lebercilin), ornithine ketoacid aminotransferase (gyrate atrophy), retinoschisin 1 (X-linked retinoschisis), USHIC (Usher's Syndrome 1C), X-linked retinitis pigmentosa GTPase (XLRP), MERTK (AR forms of RP: retinitis pigmentosa), DFNBI (connexin 26 deafness), ACHM 2, 3 and 4 (achromatopsia), PKD-1 or PKD-2 (polycystic kidney disease), TPP1, CLN2, a sulfatase, N-acetylglucosamine-1-phosphate transferase, cathepsin A, GM2-AP, NPC1, VPC2, a sphingolipid activator protein, one or more zinc finger nucleases for genome editing, or one or more donor sequences used as repair templates for genome editing.

Nucleic acid molecules, vectors such as cloning, expression vectors (e.g., vector genomes) and plasmids, may be prepared using recombinant DNA technology methods. The availability of nucleotide sequence information enables preparation of nucleic acid molecules by a variety of means. For example, a heterologous nucleic acid encoding Factor IX (FIX) comprising a vector or plasmid can be made using various standard cloning, recombinant DNA technology, via cell expression or in vitro translation and chemical synthesis techniques. Purity of polynucleotides can be determined through sequencing, gel electrophoresis and the like. For example, nucleic acids can be isolated using hybridization or computer-based database screening techniques. Such techniques include, but are not limited to: (1) hybridization of genomic DNA or cDNA libraries with probes to detect homologous nucleotide sequences: (2) antibody screening to detect polypeptides having shared structural features, for example, using an expression library: (3) polymerase chain reaction (PCR) on genomic DNA or cDNA using primers capable of annealing to a nucleic acid sequence of interest: (4) computer searches of sequence databases for related sequences; and (5) differential screening of a subtracted nucleic acid library.

Methods that are known in the art for generating rAAV virions: for example, transfection using AAV vector and AAV helper sequences in conjunction with coinfection with one AAV helper viruses (e.g., adenovirus, herpesvirus, or vaccinia virus) or transfection with a recombinant AAV vector, an AAV helper vector, and an accessory function vector. Non-limiting methods for generating rAAV virions are described, for example, in U.S. Pat. Nos. 6,001,650 and 6,004,797, International Application PCT/US 16/64414 (published as WO 2017/096039) and U.S. Provisional Application Nos. 62/516,432 and 62/531,626. Following recombinant rAAV vector production (i.e., vector generation in cell culture systems), rAAV virions can be obtained from the host cells and cell culture supernatant and then purified as set forth herein.

Methods of Purification of Full rAAV Particles

In one general aspect, the application relates to a method for purifying full recombinant adeno-associated virus (rAAV) particles, the method comprising:
(a). providing an rAAV preparation comprising the full rAAV particles and non-full particles;
(b). loading the rAAV preparation in a loading buffer to a column comprising a chromatography medium, wherein the full rAAV particles have a higher binding affinity to the chromatography medium than the non-full particles; and
(c). eluting the full rAAV particles bound to the chromatography medium with an elution buffer to obtain a purified preparation comprising an increased ratio of the full rAAV particles to the non-full particle.

According to the embodiments of the application, the rAAV preparation can be any mixture that comprises full rAAV particles and non-full particles, including empty and partial particles, and variants thereof. The rAAV preparation can also comprise a mixture of various non-full particles (e.g. empty and partial particles) for further separation. For example, the rAAV preparation can be a cell lysate, a processed cell lysate, supernatant, or previously purified preparation. An rAAV preparation useful for a method of the application can be obtained using any method of collecting rAAV known in the art in view of the present disclosure. For example, a cell lysate can be obtained by disrupting or lysing the cells and removing the cell debris by centrifugation, microfluidization, and/or depth filtration. The cell lysate can be used directly or it can be further processed or stored before being used for a method of the application.

Typically, the cell lysate is clarified to remove cell debris, such as filtering and centrifuging, to render a clarified cell lysate. The lysate (optionally clarified) contains full rAAV particles, non-full particles, and other rAAV vector production/process related impurities, such as soluble cellular components from the host cells that can include, inter alia, cellular proteins, lipids, and/or nucleic acids, and cell culture medium components. The optionally clarified lysate can be then subjected to additional purification steps to remove the other process related impurities by any method known in the art. The resulting processed lysate can be diluted or concentrated with an appropriate buffer before being used for a method of the application.

In some embodiments, the non-full particles comprise empty particles.

In some embodiments, the non-full particles comprise partial particles.

In some embodiments, the non-full particles comprise both empty particles and partial particles.

In some embodiments, the chromatography medium is an ion exchange column chromatography medium, preferably an anion exchange chromatography medium. The anion exchange chromatography medium used in this method can be a strong anion exchange resin or a week anion exchange resin. Preferably, the anion exchange chromatography medium comprises an anion exchange ligand such as proprietary quaternary amine, quarternized polyethyleneimine, polyethyleneimine, or dimethylaminopropyl. More preferably, the anion exchange chromatography medium is selected from a weak anion exchange resin (e.g., Poros 50 D, Poros 50 PI) or a strong anion exchange resin (e.g., Poros XQ, Poros 50 HQ). Other examples of anion exchange chromatography medium include, but are not limited to, DEAE Sepharose FF, Q-Sepharose (HP and FF), Q Sepharose FF (low and high substituted), Capto Q, Q XP, Source 30 Q and 15 Q, Fractogel DEAE and MPHQ. Other examples of anion exchange chromatography medium include monolith such as CIMmultus™ QA Monolithic Column.

In some embodiments, the chromatography medium is an ion exchange chromatography medium, preferably an anion exchange chromatography medium.

In some embodiments, the column chromatography medium is selected from the group consisting of Poros 50 HQ, Poros 50 D, Poros 50 PI, Capto ImpRes Q, and Poros XQ, preferably Poros XQ.

In some embodiments, the chromatography medium is a monolith such as CIMmultus™ QA Monolithic Column.

In some embodiments, multiple chromatography media are used.

In some embodiments, where multiple chromatography media are used, the media is the same.

In some embodiments, the ion exchange chromatography medium is used in conjunction with (meaning prior to or subsequent to) affinity chromatography medium such as AVB Sepharose™ High Performance (GE Healthcare, Marlborough. MA), or size-exclusion chromatography such as Superdex 200 (GE Healthcare).

In some embodiments, when an rAAV preparation is loaded in a loading buffer to the column comprising a chromatography medium in step (b), only the full rAAV particles bind to the chromatography medium, while the non-full particles do not bind to the chromatography medium and flow thorough the column. In some embodiments, the flowthrough from the column comprises empty particles. In some embodiments, the flowthrough from the column comprises partial particles. In some embodiments, the flowthrough from the column comprises both the full rAAV particles and empty particles. In some embodiments, the flowthrough from the column comprises both the full rAAV particles and partial particles. In some embodiments, the flowthrough from the column comprises both the full rAAV particles and non-full particles, including empty and partial particles.

In some embodiments, when an rAAV preparation in a loading buffer is applied to the column comprising a chromatography medium in step (b), both the full rAAV particles and non-full particles, including variants like partials, bind to the chromatography medium, but the full rAAV particles have a higher binding affinity to the chromatography medium than the non-full particles; and the quantity of the full and non-full particles applied to the column exceeds the binding capacity of the chromatography medium, such that non-full particles bound to the chromatography medium are displaced by the full rAAV particles into a load flowthrough from the column. The load flowthrough from the column comprises both full rAAV particles and non-full particles. In certain embodiments, some non-full particles (e.g. empty and/or partial particles) bind to the chromatography medium, which can be eluted by a wash buffer before the elution of the full rAAV particles with an elution buffer.

While not wishing to be bound by theory, it is believed that the separation of full and non-full AAV particles occurs during loading due to the displacement phenomenon. When a rAAV preparation comprising a mixture of particles is first applied to a column, all particles bind the available sites on the column. As the available sites are occupied towards the entrance of the column, the full rAAV particles, which have a higher affinity to the chromatography medium, displace the bound non-full particles, which have a lower affinity to the chromatography medium, and the full particles become enriched at the top portion of the column. In the process the non-full particles bind to the chromatography medium downstream. When more of the mixture is applied to the column, more of the full rAAV particles will bind to the chromatography medium upstream, displacing the bound non-full particles and pushing the non-full particles further downstream. In certain embodiment, after the sample loading is completed, no non-full particles or a reduced amount of non-full particles are bound to the chromatography medium compared to that of the full rAAV. Because the displacement of the non-full particles by the full particles is a result of their differing affinities to the chromatography medium, enhancing the contrast in their affinities can improve separation. The affinity of particles binding to the chromatography medium is dictated by factors such as those pertaining to the rAAV particles, resin, and the environment (including buffer conditions (salt and pH)). Similarly, this reasoning applies to partial particles when the non-full rAAV particles comprise partial particles. As variants of the non-full particle population, partial particles, may bind the chromatography medium with higher affinity than the other non-full particles, particularly empty particles, but with less affinity than the full particles, and thus both the full and partial particles may displace the other non-full particles. Enhancing the contrast in the affinities of the full and partial particles to the chromatography medium, in addition to the other non-full particles, can further improve separation of the partial particles from the full particles. Also similarly, when the non-full rAAV particles comprise empty particles, the disclosed method can be used to improve separation of the empty particles from the full and other non-full particles.

The benefit of displacement is realized once a breakthrough occurs in a column, surpassing the point of binding capacity of the column chromatography medium. As the chromatography medium resin proceeds beyond its saturation locally, the competition between full particles and non-full particles ensues, leading to the displacement of bound non-full particles by incoming full particles.

The benefit of displacement can be similarly applied to separation of other rAAV impurities and product variants, such as partially filled capsids with truncated transgene, empty, partial or full capsid variants with post-translational modifications, or fragments or aggregates, based on their different affinities to chromatography medium.

In some embodiments, when the non-full particles comprise both empty particles and partial particles, all non-full particles bind on the available sites on the column. As the available sites are occupied, the partial particles, which have a relatively higher affinity to the chromatography medium than the empty particles, displace the bound empty particles.

A Chromatography medium such as anion exchange resin can be equilibrated, washed, and eluted with various buffers under various conditions such as pH, and buffer volumes. The following is intended to describe particular nonlimiting examples, but is not intended to limit the invention.

Anion exchange chromatography can be equilibrated using standard buffers and according to the manufacturer's specifications. After equilibration, sample is then loaded. Subsequently, the chromatography medium is washed at least once, or more, e.g., 2-10 times the column volume. Elution from the chromatography medium is by way of a high salt buffer, for 2-20 column volumes.

According to embodiments of the application, equilibration buffers and solutions for washes and elutions for anion exchange chromatography are appropriate at pH from about pH 6.0 to pH 12. In addition, appropriate equilibration buffers and solutions for washes and elutions for anion exchange columns are generally cationic or zwitterionic in nature. Such buffers include, without limitation, buffers with the following buffer agents; N-methylpiperazine; piperazine; Bis-Tris; Bis-Tris propane; Triethanolamine; Tris; Tris acetate; N-methyldiethanolamine: 1,3-diaminopropane; ethanolamine; acetic acid, and the like. To elute the sample, the ionic strength of the starting buffer is increased using a salt, such as NaCl, KCl, MgCl₂, CaCl₂), sulfate, formate or acetate.

In some embodiments, the loading buffer comprises at least one buffer selected from the group consisting of Tris, Bis-tris, Bis-tris propane, Tris acetate, ethanolamine, and phosphate. In certain embodiments, the loading buffer comprised the selected buffer at a concentration of about 20-50 mM, such as 20 mM, 30 mM, 40 mM, 50 mM, 100 mM or any concentration in between. In preferred embodiment, the loading buffer comprises 20-50 mM Tris.

In some embodiments, the loading buffer comprises at least one salt of a cation selected from the group consisting of K(I), Li(I), Ca(II), Mg(II), Cu(II), Ba(II)), Co(II), Ni(II), Mn(II), Zn(II), Cd(II), Pb(II), Fe(III), Fe(II), Na(I), and NH₄⁺. For example, the loading buffer can comprise multiple salts selected from the group consisting of NaCl, MgCl₂, and CaCl₂). In preferred embodiments, the loading buffer comprises NaCl, MgCl₂, CuCl₂, LiCl, and CaCl₂). The anionic component to the salt is not determinative and no particular anion is preferred.

In some embodiments, the loading buffer contains at least one salt of a cation selected from the group consisting of K(I), Li(I), Ca(II), Mg(II), Cu(II), Ba(II)), Co(II), Ni(II), Mn(II), Zn(II), Cd(II), Pb(II), Fe(III), Fe(II), Na(I), and NH₄⁺.

In some embodiments, when the loading buffer comprises at least one salt of a cation selected from the group consisting of K(I), Li(I), Ca(II), Mg(II), Cu(II), Ba(II)), Co(II), Ni(II), Mn(II), Zn(II), Cd(II), Pb(II), Fe(III), Fe(II), Na(I), and NH₄⁺, only the full rAAV particles bind to the chromatography medium, while the non-full particles do not bind to the chromatography medium and flow thorough the column. In preferred embodiments, the loading buffer comprises at least CaCl₂).

In some embodiments, the loading buffer comprises about 10-100 mM sodium salt, such as 10, 20, 30, 40, 50, 60, 70, 80, 90, 100 mM, or any concentration in between. In preferred embodiments, the concentration of sodium salt is between about 20-60 mM, such as 20, 25, 30, 35, 40, 45, 50, 55, 60 mM, or any concentration in between.

In some embodiments, the loading buffer comprises about 0-20 mM magnesium salt, such as 0, 5, 10, 15, 20 mM, or any concentration in between. In preferred embodiments, the concentration of magnesium salt is between about 1-10 mM, such as 1, 2, 3, 4, 5, 6, 7, 8, 9, 10 mM, or any concentration in between.

In some embodiments, the loading buffer comprises about 0-100 mM lithium salt, such as 0, 20, 30, 40, 50, 60, 70, 80, 90, 100 mM, or any concentration in between. In preferred embodiments, the concentration of lithium salt is between about 0-75 mM, such as 0, 15, 25, 35, 45, 55, 65, 75 mM, or any concentration in between.

In some embodiments, the loading buffer comprises about 0-10 mM calcium salt, such as 0, 1, 2, 3, 4, 5, 6, 7, 8, 9, 10 mM, or any concentration in between. In preferred embodiments, the concentration of calcium salt is between about 0.1-2.5 mM, such as 0.1, 0.5, 1.0. 1.5, 2.0, 2.5 mM, or any concentration in between.

In some embodiments, the loading buffer comprises about 0-5 mM copper salt, such as 0, 1, 1.5, 2, 2.5, 3, 3.5, 4, 4.5, 5 mM, or any concentration in between. In preferred embodiments, the concentration of copper salt is between about 0.1-2.5 mM, such as 0.1, 0.5, 1.0. 1.5, 2.0, 2.5 mM, or any concentration in between.

In some embodiments, the loading buffer comprises about 5-100 mM ammonium salt, such as 5, 10, 15, 20, 25, 30, 35, 40, 45, 50, 60, 70 80, 90, 100 mM, or any concentration in between. In preferred embodiments, the concentration of ammonium is between about 10-50 mM, such as 10, 15, 20, 25, 30, 35, 40, 45, 50 mM, or any concentration in between.

In some embodiments, the loading buffer contains at least one salt of a cation selected from the group consisting of K(I), Li(I), Ca(II), Mg(II), Cu(II), Ba(II)), Co(II), Ni(II), Mn(II), Zn(II), Cd(II), Pb(II), Fe(III), Fe(II), Na(I), and NH₄.

In some embodiments, the loading buffer has a pH of about 7-10, such as 7, 7.5, 8, 8.5, 9, 9.5, 10, or any pH in between, preferably at pH about 8-9.

In certain embodiment, the loading buffer comprises about 20-60 mM NaCl, 1-5 mM MgCl₂, 0.1-2.5 mM CaCl₂), preferably in Tris, more preferably in 20-50 mM Tris.

In certain embodiments, the loading buffer comprises about 20-60 mM NaCl, 10-30 mM (NH₄)₂SO₄ 1-5 mM MgCl₂, 0.1-2.5 mM CuCl₂, preferably in Tris, more preferably in 20-100 mM Tris.

In some embodiments, the loading buffer comprises at least one surfactant.

In some embodiments, the surfactant in the loading buffer is selected from the group consisting of poloxamer 188, polysorbate 80, polysorbate 20, NP-40, Triton X-100, and Triton CG-110.

In some embodiments, the concentration of the surfactant in the loading buffer is between 0.0001% to 0.1%.

After loading step (b), loading the rAAV preparation in a loading buffer to a column comprising a chromatography medium, wherein the full rAAV particles have a higher binding affinity to the chromatography medium than the non-full particles, the full rAAV particles bind to the chromatography medium. The column can be optionally washed with a wash buffer before the elution. For example, the wash buffer can have increased salt concentration and/or increased pH for elution as compared to the loading conditions, in order to remove the non-full particles bound to the chromatography medium.

Then the full particles bound to the chromatography medium are eluted in purer form, e.g., using increased salt concentration and/or adjusted pH for elution as compared to the loading conditions. Adjusting pH can enhance separation of full and non-full particles. Preferably, the elution of full particles is achieved with salt gradient alone, without changing the pH.

In some embodiments, the elution buffer comprises at least one buffer selected from the group consisting of Tris, Bis-tris, Bis-tris propane, Tris acetate, ethanolamine, and phosphate. In certain embodiments, the elution buffer comprised the selected buffer at a concentration of about 20-70 mM, such as 20 mM, 30 mM, 40 mM, 50 mM, 60 mM, 70 mM, or any concentration in between. In a preferred embodiment, the elution buffer comprises 40-60 mM Tris.

In some embodiments, the elution buffer comprises at least one salt of a cation selected from the group consisting of K(I), Li(I), Ca(II), Mg(II), Cu(II), Ba(II)), Co(II), Ni(II), Mn(II), Zn(II), Cd(II), Pb(II), Fe(III), Fe(II), Na(I), and NH₄⁺. For example, the elution buffer can comprise one or more salts, preferably selected from the group consisting of NaCl, MgCl₂, LiCl, CuCl₂ and CaCl₂). The anionic component of the salt is not determinative.

In some embodiments, the elution buffer contains at least one salt of a cation selected from the group consisting of K(I), Li(I), Ca(II), Mg(II), Cu(II), Ba(II)), Co(II), Ni(II), Mn(II), Zn(II), Cd(II), Pb(II), Fe(III), Fe(II), Na(I), and NH₄⁺.

In some embodiments, the elution buffer comprises about 0-1000 mM NaCl, such as 0, 100, 200, 300, 400, 500, 600, 700, 800, 900, 1000 mM, or any concentration in between. In preferred embodiments, the concentration of NaCl is between about 20-300 mM, such as 20, 30, 40, 50, 60, 70, 80, 90, 100, 110, 120, 130, 140, 150, 200, 250, 300 mM, or any concentration in between.

In some embodiments, the elution buffer comprises about 0-30 mM MgCl₂, such as 0, 5, 10, 15, 20, 25, 30 mM, or any concentration in between. In preferred embodiments, the concentration of MgCl₂ is between about 2-15 mM, such as 2, 3, 4, 5, 6, 7, 8, 9, 10, 11, 12, 13, 14, 15 mM, or any concentration in between.

In some embodiments, the elution buffer comprises about 0-200 mM LiCl, such as 0, 20, 30, 40, 50, 60, 70, 80, 90, 100, 110, 120, 130, 140, 150, 160, 170, 180, 190, 200 mM, or any concentration in between. In preferred embodiments, the concentration of LiCl is between about 0-150 mM, such as 0, 20, 30, 40, 50, 60, 70, 80, 90, 100, 110, 120, 130, 140, 150 mM, or any concentration in between.

In some embodiments, the elution buffer comprises about 0.1-20 mM CaCl₂), such as 0.1, 1, 2, 3, 4, 5, 6, 7, 8, 9, 10, 12, 14, 16, 18, 20 mM, or any concentration in between. In preferred embodiments, the concentration of CaCl₂) is between about 5-10 mM, such as 5, 6, 7, 8, 9, 10 mM, or any concentration in between.

In some embodiments, the elution buffer comprises about 0-10 mM CuCl₂, such as 0, 1, 2, 3, 4, 5, 6, 7, 8, 9, 10 mM, or any concentration in between. In preferred embodiments, the concentration of CuCl₂ is between about 0-3 mM, such as 0, 1, 2, 3 mM, or any concentration in between.

In some embodiments, the elution buffer comprises about 5-100 mM (NH₄)₂SO₄, such as 5, 10, 20, 30, 40, 50, 60, 70, 80, 90, 100 mM, or any concentration in between. In preferred embodiments, the concentration of (NH₄)₂SO₄ is between about 10-50 mM, such as 10, 15, 20, 25, 30, 35, 40, 45, 50 mM, or any concentration in between.

In some embodiments, the elution buffer has a pH of about 6-10, such as 6, 6.5, 7, 7.5, 8, 8.5, 9, 9.5, 10, or any pH in between, preferably at pH about 7-9.

In certain embodiments, the elution buffer comprises about 20-150 mM NaCl, preferably in Tris, more preferably in 40-60 mM Tris.

In some embodiments, the elution buffer comprises at least one surfactant.

In some embodiments, the surfactant in the elution buffer is selected from the group consisting of poloxamer 188, polysorbate 80, polysorbate 20, NP-40, Triton X-100, and Triton CG-110.

In some embodiments, the concentration of the surfactant in the elution buffer is between 0.0001% to 0.1%.

In another general aspect, the application relates to a method for purifying full recombinant adeno-associated virus (rAAV) particles, the method comprising:
(a). providing an rAAV preparation comprising the full rAAV particles and non-full particles;
(b). loading a first batch of the rAAV preparation in a loading buffer to a first column comprising a first chromatography medium, wherein the full rAAV particles have a higher binding affinity to the first chromatography medium than the non-full particles, and the quantity of the full rAAV particles and non-full particles applied to the first column exceeds the binding capacity of the first chromatography medium such that the non-full particles bound to the first chromatography medium are displaced by the full rAAV particles into a first load flowthrough from the first column;
(c). loading the first load flowthrough to a second column comprising a second chromatography medium to obtain a loaded or partially loaded second column and first load flowthrough from the second column, wherein the second chromatography medium is the same or same type as the first chromatography medium;
(d). optionally, washing the first column with a wash buffer to obtain a washed first column;
(e). bypassing the second column after the step (c) or after the wash step of (d) if the wash step (d) is performed, and eluting the full rAAV particles bound to the first chromatography medium with an elution buffer to obtain a first eluate from the first column and an eluted first column, wherein the first eluate comprises an increased ratio of the full rAAV particles to the non-full rAAV particles;
(f). optionally, if the second column is not saturated (i.e. loaded) after the first column flowthrough, a second batch of the rAAV preparation in loading buffer can be applied to the partially loaded second column, wherein the quantity of the full rAAV particles and non-full particles applied to the second column exceeds the binding capacity of the second chromatography medium such that the non-full particles bound to the second chromatography medium are displaced by the full rAAV particles into a second load flowthrough from the second column;
(g). if step (f) is performed, loading the second load flowthrough from the second column to the washed first column between steps (d) and (e) to obtain a second loaded first column, and optionally washing second loaded first column before continuing to elute in step (e);
(h). optionally, washing the second column with a wash buffer to obtain a washed second column;
(i). bypassing the first column after the step (g) or after the wash step of (h) if the wash step (h) is performed, and eluting the full rAAV particles bound to the second chromatography medium with an elution buffer to obtain a second eluate and an eluted second column, wherein the second eluate comprises an increased ratio of the full rAAV particles to the non-full rAAV particles; and
(j). combining the first eluate and second eluate to produce a purified preparation of full rAAV particles.

In some embodiments, the non-full particles comprise empty particles.

In some embodiments, the non-full particles comprise partial particles.

In some embodiments, the non-full particles comprise both empty particles and partial particles.

In some embodiments, the first chromatography medium and/or the second chromatography medium is an ion exchange column chromatography medium, preferably an anion exchange chromatography medium.

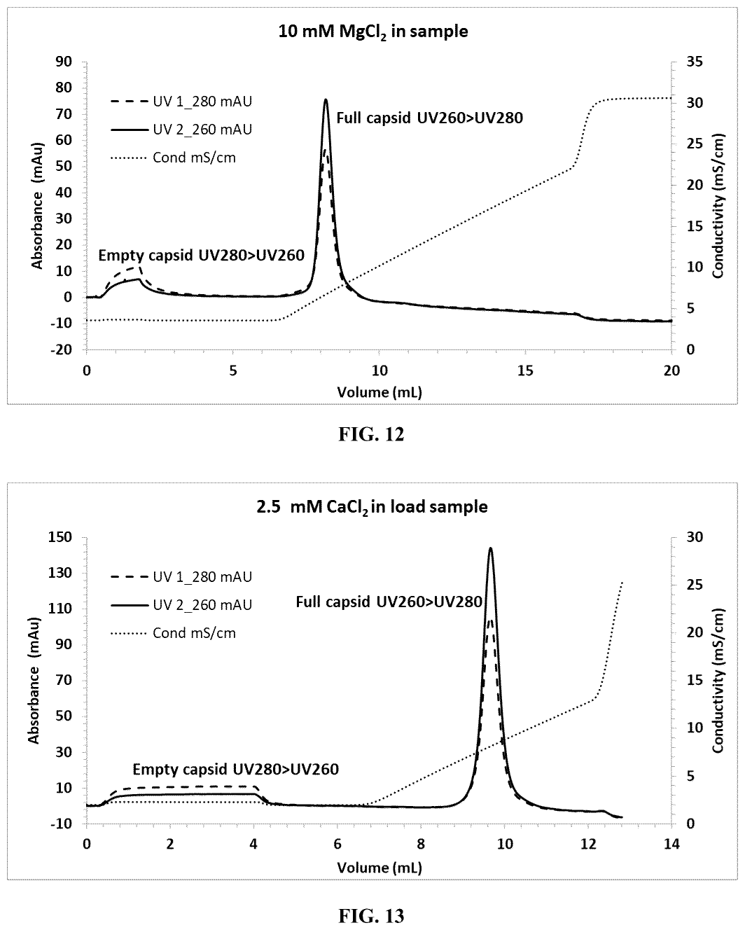

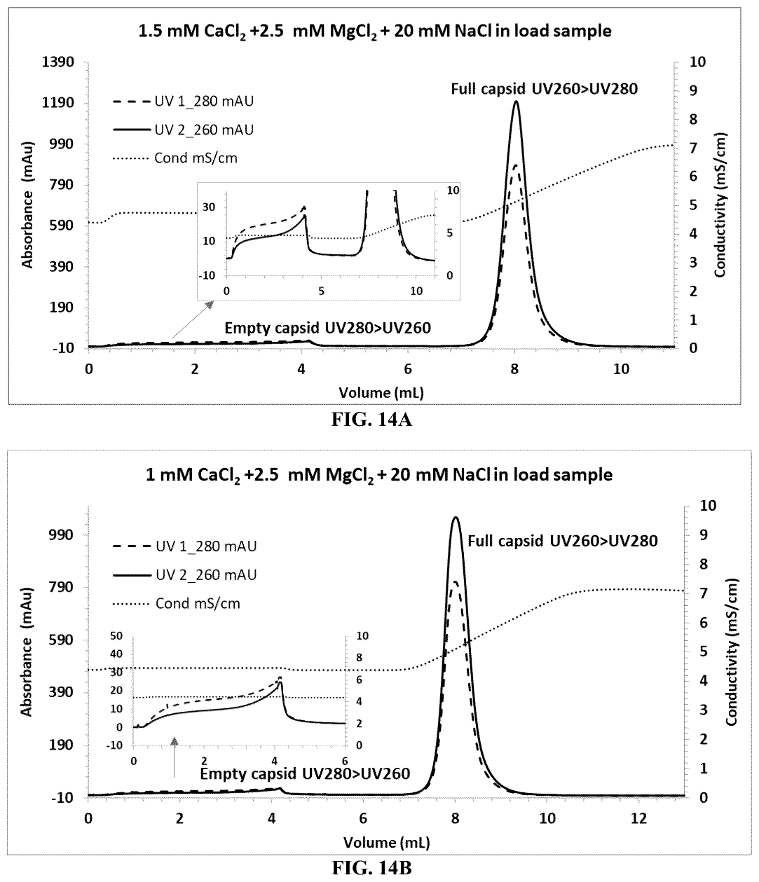

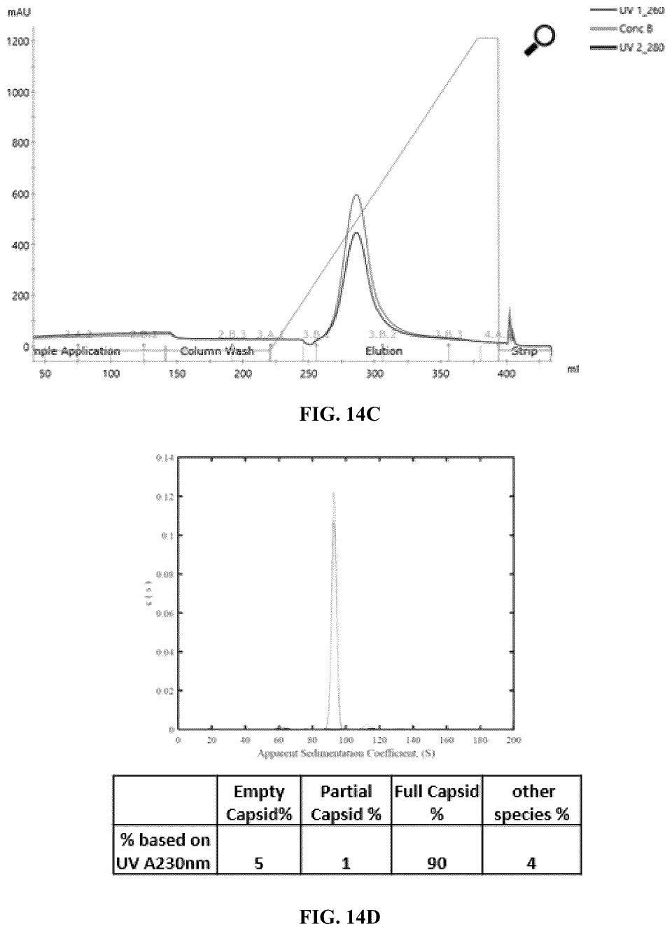

In some embodiments, the two-column purification process is set up and operated according to the implementation scheme as shown in FIG. 12. Steps (a) and (b) of the two-column process is similar to the first two steps of the one-column displacement chromatography process. After these two steps, the full rAAV particles bind to the first chromatography medium, and the first load flowthrough from the first column comprises both the full rAAV particles and non-full particles.

In step (f), a second batch of the rAAV preparation in loading buffer is applied to the second column which was partially loaded in step (c). The amount of the second batch of the rAAV preparation can be the same as the first batch, or different from the first batch. Preferably, the amount of the second batch is the same as the first batch.

According to the embodiments of the application, the quantity of the full rAAV particles and non-full particles applied to the second column in step (f) includes the quantity from the first load flowthrough and the quantity from the second batch of the rAAV preparation, and exceeds the binding capacity of the second chromatography medium. During the loading of the second batch to the partially loaded second column, the non-full particles bound to the second chromatography medium are displaced by the full rAAV particles into a second load flowthrough from the second column.

In the subsequent step (g), the second load flowthrough from the second column is loaded to the previously washed or eluted first column. The first column is then partially loaded after step (g), and the quantity of the full rAAV particles and non-full particles in the second load flowthrough from the second column does not exceed the binding capacity of the first chromatography medium at this point.

The steps (b) to (i) can be performed with one cycle or more cycles, depending on the amount of the rAAV preparation. The term “one cycle” as used herein refers to the steps from step (b) to step (i) which are performed sequentially once. The term “more cycles” as used herein refers to the steps from step (b) to step (i) which are performed sequentially more than once.

When the steps (b) to (i) are be performed with more cycles, the “first batch” in the repeated step (b) refers to a new “first batch”, that of the cycle number, of the rAAV preparation, and the “second batch” in the repeated step (f) refers to a new “second batch” of the rAAV preparation in that same cycle.

In some embodiments, when the non-full particles comprise both empty particles and partial particles, the second eluate in step (i) comprises an increased ratio of full and partial rAAV particles to the empty rAAV particles.

In some embodiments, when multiple cycles are performed, the eluted column can be subject to subsequent steps beneficial or necessary to maintain consistent column binding capacity throughout the cycles before the next cycle of loading. For example, such steps include, but are not limited to, stripping the column, cleaning and/or sanitizing the column, and/or re-equilibrating the column.

In some embodiments, the loading buffer comprises at least one buffer selected from the group consisting of Tris, Bis-tris, Bis-tris propane, Tris acetate, ethanolamine, and phosphate. In certain embodiments, the loading buffer comprised the selected buffer at a concentration of about 20-50 mM, such as 20 mM, 30 mM, 40 mM, 50 mM, or any concentration in between. In preferred embodiment, the loading buffer comprises 20-50 mM Tris.

In some embodiments, the loading buffer comprises at least one salt of a cation selected from the group consisting of K(I), Li(I), Ca(II), Mg(II), Cu(II), Ba(II)), Co(II), Ni(II), Mn(II), Zn(II), Cd(II), Pb(II), Fe(III), Fe(II), Na(I), and NH₄⁺ in the range of 0-200 mM. The anionic component to the salt is not determinant and no particular anion is preferred. For example, the loading buffer can comprise one or more salts, preferably selected from the group consisting of NaCl, MgCl₂, LiCl, CuCl₂, and CaCl₂).

In some embodiments, the loading buffer comprises about 10-100 mM sodium salt, such as 10, 20, 30, 40, 50, 60, 70, 80, 90, 100 mM, or any concentration in between. In preferred embodiments, the concentration of sodium salt is between about 20-60 mM, such as 20, 25, 30, 35, 40, 45, 50, 55, 60 mM, or any concentration in between.

In some embodiments, the loading buffer comprises about 0-20 mM magnesium salt, such as 0, 5, 10, 15, 20 mM, or any concentration in between. In preferred embodiments, the concentration of magnesium salt is between about 1-10 mM, such as 1, 2, 3, 4, 5, 6, 7, 8, 9, 10 mM, or any concentration in between.

In some embodiments, the loading buffer comprises about 0-100 mM lithium salt, such as 0, 20, 30, 40, 50, 60, 70, 80, 90, 100 mM, or any concentration in between. In preferred embodiments, the concentration of lithium salt is between about 0-75 mM, such as 0, 15, 25, 35, 45, 55, 65, 75 mM, or any concentration in between.

In some embodiments, the loading buffer comprises about 0-10 mM calcium salt, such as 0, 1, 2, 3, 4, 5, 6, 7, 8, 9, 10 mM, or any concentration in between. In preferred embodiments, the concentration of calcium salt is between about 0.1-2.5 mM, such as 0.1, 0.5, 1.0. 1.5, 2.0, 2.5 mM, or any concentration in between.

In some embodiments, the loading buffer comprises about 0-5 mM copper salt, such as 0, 1, 1.5, 2, 2.5, 3, 3.5, 4, 4.5, 5 mM, or any concentration in between. In preferred embodiments, the concentration of copper salt is between about 0.1-2.5 mM, such as 0.1, 0.5, 1.0. 1.5, 2.0, 2.5 mM, or any concentration in between.

In some embodiments, the loading buffer comprises about 5-100 mM ammonium salt, such as 5, 10, 15, 20, 25, 30, 35, 40, 45, 50, 60, 70, 80, 90, 100 mM, or any concentration in between. In preferred embodiments, the concentration of ammonium is between about 10-50 mM, such as 10, 15, 20, 25, 30, 35, 40, 45, 50 mM, or any concentration in between.

In some embodiments, the loading buffer comprises at least one surfactant.

In some embodiments, the surfactant in the loading buffer is selected from the group consisting of poloxamer 188, polysorbate 80, polysorbate 20, NP-40, Triton X-100, and Triton CG-110.

In some embodiments, the concentration of the surfactant in the loading buffer is between 0.0001% to 0.1%.

In some embodiments, the loading buffer has a pH of about 6-10, such as 6, 6.5, 7, 7.5, 8, 8.5, 9, 9.5, 10, or any pH in between, preferably at pH about 8-9.

In certain embodiment, the loading buffer comprises about 20-60 mM NaCl, 1-5 mM MgCl₂, 0.1-2.5 mM CaCl₂), preferably in Tris, more preferably in 20-50 mM Tris.

In certain embodiments, the loading buffer comprises about 20-60 mM NaCl, 5-30 mM (NH₄)₂SO₄, 1-5 mM MgCl₂, 0.1-3 mM CuCl₂, preferably in Tris, more preferably in 20-50 mM Tris, with 0.00005-0.01% poloxamer 188, more preferably with 0.0002-0.001% poloxamer 188.

After the loading step, the full particles bind to the chromatography medium, and the non-full particles exit the column in the flowthrough. The column can be optionally washed with a suitable wash buffer before the elution. For example, the wash buffer can have increased salt concentration and/or increased pH for elution as compared to the loading conditions, in order to remove the non-full particles bound to the chromatography medium.

Then the full particles bound to the chromatography medium are subsequently eluted in purer form, e.g., using increased salt concentration and/or adjusted pH for elution as compared to the loading conditions. Adjusting the pH can allow for enhanced separation. Preferably, the elution of full particles is achieved with salt gradient alone, without increasing the pH.

In some embodiments, the elution buffer comprises at least one buffer selected from the group consisting of Tris, Bis-tris, Bis-tris propane Tris acetate, ethanolamine, and phosphate. In certain embodiments, the loading buffer comprised the selected buffer at a concentration of about 20-70 mM, such as 20 mM, 30 mM, 40 mM, 50 mM, 60 mM, 70 mM, or any concentration in between. In a preferred embodiment, the loading buffer comprises 40-60 mM Tris.

In some embodiments, the elution buffer comprises at least one salt of a cation selected from the group consisting of K(I), Li(I), Ca(II), Mg(II), Cu(II), Ba(II)), Co(II), Ni(II), Mn(II), Zn(II), Cd(II), Pb(II), Fe(III), Fe(II), Na(I), and NH₄⁺. For example, the elution buffer can comprise one or more salts, preferably selected from the group consisting of NaCl, MgCl₂, LiCl, CuCl₂ and CaCl₂). The anionic component of the salt is not determinative

In some embodiments, the elution buffer contains at least one salt of a cation selected from the group consisting of K(I), Li(I), Ca(II), Mg(II), Cu(II), Ba(II)), Co(II), Ni(II), Mn(II), Zn(II), Cd(II), Pb(II), Fe(III), Fe(II), Na(I), and NH₄⁺. For example, the loading buffer can comprise one or more salts, preferably selected from the group consisting of NaCl, MgCl₂, LiCl, CuCl₂, and CaCl₂).

In some embodiments, the elution buffer comprises about 0-1000 mM sodium salt, such as 0, 100, 200, 300, 400, 500, 600, 700, 800, 900, 1000 mM, or any concentration in between. In preferred embodiments, the concentration of sodium salt is between about 20-300 mM, such as 20, 30, 40, 50, 60, 70, 80, 90, 100, 110, 120, 130, 140, 150, 200, 250, 300 mM, or any concentration in between.

In some embodiments, the elution buffer comprises about 0-30 mM magnesium salt, such as 0, 5, 10, 15, 20, 25, 30 mM, or any concentration in between. In preferred embodiments, the concentration of magnesium salt is between about 2-15 mM, such as 2, 3, 4, 5, 6, 7, 8, 9, 10, 11, 12, 13, 14, 15 mM, or any concentration in between.

In some embodiments, the elution buffer comprises about 0-200 mM lithium salt, such as 0, 20, 30, 40, 50, 60, 70, 80, 90, 100, 110, 120, 130, 140, 150, 160, 170, 180, 190, 200 mM, or any concentration in between. In preferred embodiments, the concentration of lithium salt is between about 0-150 mM, such as 0, 20, 30, 40, 50, 60, 70, 80, 90, 100, 110, 120, 130, 140, 150 mM, or any concentration in between.

In some embodiments, the elution buffer comprises about 0.1-20 mM calcium salt, such as 0.1, 1, 2, 3, 4, 5, 6, 7, 8, 9, 10, 12, 14, 16, 18, 20 mM, or any concentration in between. In preferred embodiments, the concentration of calcium salt is between about 5-10 mM, such as 5, 6, 7, 8, 9, 10 mM, or any concentration in between.

In some embodiments, the elution buffer comprises about 0-5 mM copper salt, such as 0, 1, 1.5, 2, 2.5, 3, 3.5, 4, 4.5, 5 mM, or any concentration in between. In preferred embodiments, the concentration of copper salt is between about 0.1-2.5 mM, such as 0.1, 0.5, 1.0. 1.5, 2.0, 2.5 mM, or any concentration in between.

In some embodiments, the elution buffer comprises about 5-100 mM ammonium salt, such as 5, 10, 15, 20, 25, 30, 35, 40, 45, 50, 60, 70, 80, 90, 100 mM, or any concentration in between. In preferred embodiments, the concentration of ammonium is between about 10-50 mM, such as 10, 15, 20, 25, 30, 35, 40, 45, 50 mM, or any concentration in between.

In some embodiments, the elution buffer has a pH of about 6-10, such as 6, 6.5, 7, 7.5, 8, 8.5, 9, 9.5, 10, or any pH in between, preferably at pH about 8-9.

In certain embodiment, the elution buffer comprises about 20-150 mM NaCl, preferably in Tris, more preferably in 40-60 mM Tris.

In some embodiments, the elution buffer comprises at least one surfactant.

In some embodiments, the surfactant in the elution buffer is selected from the group consisting of poloxamer 188, polysorbate 80, polysorbate 20, NP-40, Triton X-100, and Triton CG-110.

In some embodiments, the concentration of the surfactant in the elution buffer is between 0.0001% to 0.1%.

In some embodiments, the method uses no less than two columns, such as three columns, for the purification of full rAAV particles. In particular, the method does not comprise steps (g) to (j), but further comprises:
(k). after step (f), loading the second load flowthrough form the second column to a third column comprising a third chromatography medium to obtain a loaded or partially loaded third column, preferably, the third chromatography medium is the same type as the first chromatography medium;
(l). optionally, washing the second column with a wash buffer to obtain a washed second column;
(m). bypassing the first column after step (k) or after the wash step (l) if the wash step (1) is performed, and eluting the full rAAV particles bound to the second chromatography medium with an elution buffer to obtain a second eluate and an eluted second column, wherein the second eluate comprises an increased ratio of the full rAAV particles to the non-full rAAV particles;
(n). optionally, if the third column is not saturated (i.e. loaded) after the second column flowthrough, a third batch of the rAAV preparation in loading buffer can be applied to the partially loaded third column, wherein the quantity of the full rAAV particles and non-full particles applied to the third column exceeds the binding capacity of the third chromatography medium such that the non-full particles bound to the third chromatography medium are displaced by the full rAAV particles into a third load flowthrough from the third column;
(o). if step (n) is performed, loading the third load flowthrough from the third column to the washed first column between steps (d) and (e) to obtain a second loaded first column, and optionally washing second loaded first column before continuing to elute in step (e);
(p). optionally, washing the third column with a wash buffer to obtain a washed third column;
(q). bypassing the first column and second column, and eluting the full rAAV particles bound to the third chromatography medium with an elution buffer to obtain a third eluate and an eluted third column, wherein the third eluate comprises an increased ratio of the full rAAV particles to the non-full rAAV particles; and
(r). combining the first eluate, second eluate, and third eluate to produce a purified preparation of full rAAV particles.

In some embodiments, when the non-full particles comprise both empty particles and partial particles, the full particles are enriched in the first column, the partial particles are enriched in the second column, and the empty particles are enriched in the third column.

To the best of the inventors' knowledge, no displacement chromatography method exists for the purification of full rAAV particles from non-full rAAV particles. Compared to existing purification method for purification of full particles, such as normal anion exchange chromatography, the purity of the full particles is increased significantly after a process of the application. In some embodiments, the purified preparation is substantially free of the non-full particles, more particularly, substantially free of empty particles and/or partial particles. In other embodiments, the purified preparation comprises an increased ratio of the full rAAV particles to the non-full particles than that of the rAAV preparation. Preferably, the ratio of the full rAAV particles to the non-full particles in the purified preparation is no less than 9:1, such as no less than 10:1, 15:1, 20:1, 25:1, 30:1, 35:1, 40:1, 45:1 or 50:1, or any ratio in between, more preferably no less than 49:1.

According to the embodiments of the application, the ratio of the full rAAV particles to the non-full particles in the purified preparation can be calculated by the number of the full rAAV particles and the non-full particles. In some embodiments, the ratio is derived from the calibration curve based on molar concentrations of the full rAAV particles and the non-full particles. In some embodiments, the ratio is calculated based on the number of the full rAAV particles and the non-full particles.

In some embodiments, the ratio of the full particles to the non-full particles in the purified full rAAV particles is no less than 9:1, preferably no less than 49:1.

The displacement chromatography method of the application can also achieve high yield/recovery of purified full particles. In some embodiments, the yield of the purified full rAAV particles is no less than 70%, preferably no less than 80%, more preferably no less than 90%, and most preferably no less than 95%. The term “yield” as used herein refers to the percentage or proportion of the full rAAV particles in the purified preparation with respect to the full rAAV particles in the initial rAAV preparation. Multiple methods to calculate yield exist. One way to calculate yield is in terms of a percent (%)=(amount of full rAAV particles in the purified preparation)/(amount of the full rAAV particles in the initial rAAV preparation), multiplied by 100. Another way to calculate percent yield is dividing the number of copies of the transgene in the purified preparation by the number of copies of the transgene in the initial preparation, and multiply by 100.

In another general aspect, the application relates to a method for purifying full recombinant adeno-associated virus (rAAV) particles comprising:
(a). providing an rAAV preparation comprising the full rAAV particles and non-full particles;
(b). loading the rAAV preparation in a loading buffer to a column comprising a chromatography medium, wherein the loading buffer comprises CaCl₂), and the full rAAV particles bind to the chromatography medium;
(c). eluting the full rAAV particles bound to the chromatography medium with an elution buffer to obtain a purified preparation, wherein the elution buffer optionally comprises CaCl₂).

In some embodiments, the chromatography medium is an ion exchange column chromatography medium, preferably an anion exchange chromatography medium.

In some embodiments, the column chromatography medium is selected from the group consisting of Poros HQ, Poros PD, polyethyleneimine (PI), Capto ImpRes Q, and Poros XQ, preferably Poros XQ.

In some embodiments, the quantity of the full rAAV particles and non-full particles applied to the column does not exceed the binding capacity of the chromatography medium.

In some embodiments, the quantity of the full rAAV particles and non-full particles applied to the column exceeds the binding capacity of the chromatography medium such that the non-full particles bound to the first chromatography medium are displaced by the full rAAV particles into a first load flowthrough from the first column.

In some embodiments, the non-full particles comprise empty particles.

In some embodiments, the non-full particles comprise partial particles.

In some embodiments, the non-full particles comprise both empty particles and partial particles.

According to the embodiments of the application, addition of salts of a cation selected from the group consisting of K(I), Li(I), Ca(II), Mg(II), Cu(II), Ba(II)), Co(II), Ni(II), Mn(II), Zn(II), Cd(II), Pb(II), Fe(III), Fe(II), Na(I), and NH₄⁺ in the loading buffer can increase the affinity difference to the chromatography medium, so that more full rAAV particles bind to the chromatography medium while less non-full particles bind to the chromatography medium. Therefore, the load flowthrough from the column contains less full particles, thus increasing the recovery yield of the full particles. The anionic component of a given salt is not determinative.

In some embodiments, the loading buffer comprises about 0-10 mM calcium salt, such as 0, 1, 2, 3, 4, 5, 6, 7, 8, 9, 10 mM, or any concentration in between. In preferred embodiments, the concentration of calcium salt is between about 0.1-2.5 mM, such as 0.1, 0.5, 1.0. 1.5, 2.0, 2.5 mM, or any concentration in between.

In some embodiments, the loading buffer comprises about 0-100 mM lithium salt, such as 0, 20, 30, 40, 50, 60, 70, 80, 90, 100 mM, or any concentration in between. In preferred embodiments, the concentration of lithium salt is between about 0-75 mM, such as 0, 15, 25, 35, 45, 55, 65, 75 mM, or any concentration in between.

In some embodiments, the loading buffer comprises at least one buffer selected from the group consisting of Tris, Bis-tris, Bis-tris propane, Tris acetate, ethanolamine, and phosphate. In certain embodiments, the loading buffer comprised the selected buffer at a concentration of about 20-50 mM, such as 20 mM, 30 mM, 40 mM, 50 mM, or any concentration in between. In preferred embodiment, the loading buffer comprises 20-50 mM Tris.

In some embodiments, the loading buffer further comprises sodium salt and/or magnesium salt. In preferred embodiments, the loading buffer further comprises both sodium and magnesium salts.

In some embodiments, the loading buffer comprises about 10-100 mM sodium salt, such as 10, 20, 30, 40, 50, 60, 70, 80, 90, 100 mM, or any concentration in between. In preferred embodiments, the concentration of sodium salt is between about 20-60 mM, such as 20, 25, 30, 35, 40, 45, 50, 55, 60 mM, or any concentration in between.

In some embodiments, the loading buffer comprises about 0-20 mM magnesium salt, such as 0, 5, 10, 15, 20 mM, or any concentration in between. In preferred embodiments, the concentration of magnesium salt is between about 1-10 mM, such as 1, 2, 3, 4, 5, 6, 7, 8, 9, 10 mM, or any concentration in between.

In some embodiments, the loading buffer has a pH of about 6-10, such as 6, 6.5, 7, 7.5, 8, 8.5, 9, 9.5, 10, or any pH in between, preferably at pH about 8-9.

In certain embodiment, the loading buffer comprises about 20-60 mM NaCl, 1-5 mM MgCl₂, 0.1-2.5 mM CaCl₂), preferably in Tris, more preferably in 20-50 mM Tris.

In some embodiments, the loading buffer comprises at least one surfactant.

In some embodiments, the surfactant in the loading buffer is selected from the group consisting of poloxamer 188, polysorbate 80, polysorbate 20, NP-40, Triton X-100, and Triton CG-110.

In some embodiments, the concentration of the surfactant in the loading buffer is between 0.0001% to 0.1%.

After the loading step, the full particles bind to the chromatography medium, and the non-full particles exit the column in the flowthrough. The column can be optionally washed with a suitable wash buffer. For example, the wash buffer can have increased salt concentration and/or adjusted pH for elution as compared to the loading conditions, in order to remove the non-full particles bound to the chromatography medium.

Then the full particles bound to the chromatography medium are subsequently eluted in purer form, e.g., using increased salt concentration and/or adjusted pH for elution as compared to the loading conditions. Adjusting pH can increase separation of the full and non-full rAAV particles. Preferably, the elution of full particles is achieved with salt gradient alone, without increasing the pH.

In some embodiments, the elution buffer comprises at least one buffer selected from the group consisting of Tris, Bis-tris, Bis-tris propane, Tris acetate, ethanolamine and phosphate.

In some embodiments, the elution buffer comprises at least one salt of a cation selected from the group consisting of K(I), Li(I), Ca(II), Mg(II), Cu(II), Ba(II)), Co(II), Ni(II), Mn(II), Zn(II), Cd(II), Pb(II), Fe(III), Fe(II), Na(I), and NH₄⁺. For example, the elution buffer can comprise one or more salts, preferably selected from the group consisting of NaCl, MgCl₂, LiCl, CuCl₂ and CaCl₂). The anionic component of the salt is not determinative.

In some embodiments, the elution buffer contains at least one salt of a cation selected from the group consisting of K(I), Li(I), Ca(II), Mg(II), Cu(II), Ba(II)), Co(II), Ni(II), Mn(II), Zn(II), Cd(II), Pb(II), Fe(III), Fe(II), Na(I), and NH₄⁺. For example, the loading buffer can comprise one or more salts, preferably selected from the group consisting of NaCl, MgCl₂, LiCl, CuCl₂, and CaCl₂

In some embodiments, the elution buffer comprises about 0.1-20 mM calcium salt, such as 0.1, 1, 2, 3, 4, 5, 6, 7, 8, 9, 10, 12, 14, 16, 18, 20 mM, or any concentration in between. In preferred embodiments, the concentration of calcium salt is between about 5-10 mM, such as 5, 6, 7, 8, 9, 10 mM, or any concentration in between.

In some embodiments, the elution buffer comprises about 0-200 mM lithium salt, such as 0, 20, 30, 40, 50, 60, 70, 80, 90, 100, 110, 120, 130, 140, 150, 160, 170, 180, 190, 200 mM, or any concentration in between. In preferred embodiments, the concentration of lithium salt is between about 0-150 mM, such as 0, 20, 30, 40, 50, 60, 70, 80, 90, 100, 110, 120, 130, 140, 150 mM, or any concentration in between.

In some embodiments, the elution buffer comprises at least one buffer selected from the group consisting of Tris, Bis-tris, Bis-tris propane, Tris acetate, and phosphate. In certain embodiments, the loading buffer comprised the selected buffer at a concentration of about 20-70 mM, such as 20 mM, 30 mM, 40 mM, 50 mM, 60 mM, 70 mM, or any concentration in between. In preferred embodiment, the loading buffer comprises 40-60 mM Tris.

In some embodiments, the elution buffer comprises at least one surfactant.

In some embodiments, the surfactant in the elution buffer is selected from the group consisting of poloxamer 188, polysorbate 80, polysorbate 20, NP-40, Triton X-100, and Triton CG-110.

In some embodiments, the concentration of the surfactant in the elution buffer is between 0.0001% to 0.1%.

In some embodiments, the elution buffer further comprises sodium and/or magnesium salt. In preferred embodiments, the loading buffer further comprises both sodium and magnesium salt. The anionic component of the salt is not determinative.

In some embodiments, the elution buffer comprises about 0-1000 mM sodium salt, such as 0, 100, 200, 300, 400, 500, 600, 700, 800, 900, 1000 mM, or any concentration in between. In preferred embodiments, the concentration of sodium salt is between about 20-150 mM, such as 20, 30, 40, 50, 60, 70, 80, 90, 100, 110, 120, 130, 140, 150 mM, or any concentration in between.

In some embodiments, the elution buffer comprises about 0-30 mM magnesium salt, such as 0, 5, 10, 15, 20, 25, 30 mM, or any concentration in between. In preferred embodiments, the concentration of magnesium salt is between about 2-15 mM, such as 2, 3, 4, 5, 6, 7, 8, 9, 10, 11, 12, 13, 14, 15 mM, or any concentration in between.

In some embodiments, the elution buffer has a pH of about 6-10, such as 6, 6.5, 7, 7.5, 8, 8.5, 9, 9.5, 10, or any pH in between, preferably at pH about 8-9.

In certain embodiments, the elution buffer comprises about 20-150 mM NaCl, preferably in Tris, more preferably in 40-60 mM Tris.

In some embodiments, the purified preparation is substantially free of the non-full particles. In other embodiments, the purified preparation comprises an increased ratio of the full rAAV particles to the non-full particles than that of the rAAV preparation. Preferably, the ratio of the full rAAV particles to the non-full particles in the purified preparation is no less than 9:1, such as no less than 10:1, 15:1, 20:1, 25:1, 30:1, 35:1, 40:1, 45:1 or 50:1, or any ratio in between, more preferably no less than 49:1.

According to the embodiments of the application, the ratio of the full rAAV particles to the non-full particles in the purified preparation can be calculated by the number of the full rAAV particles and the non-full particles. In some embodiments, the ratio is derived from the calibration curve based on molar concentrations of the full rAAV particles and the non-full particles. In some embodiments, the ratio is calculated based on the number of the full rAAV particles and the non-full particles.

In some embodiments, the ratio of the full particles to the non-full particles in the purified full rAAV particles is no less than 9:1, preferably no less than 49:1.

In some embodiments, the yield of the purified full rAAV particles is no less than 70%, preferably no less than 80%, more preferably no less than 90%, and most preferably not less than 95%. The term “yield” as used herein refers to the percentage of the full rAAV particles in the purified preparation with respect to the full rAAV particles in the initial rAAV preparation. For example, the yield (%)=(amount of full rAAV particles in the purified preparation)/(amount of the full rAAV particles in the initial rAAV preparation), as a percent.

The following embodiments apply to each of the general aspects disclosed herein, including the above-described general aspects.

In some embodiments, the full rAAV particles comprise a transgene that encodes a polypeptide, nucleic acid that encodes a protein or is transcribed into a transcript of interest, or nucleic acid, selected from the group consisting of a siRNA, an antisense molecule, miRNA a ribozyme and a shRNA.

The various embodiments disclosed herein are applicable to any rAAV or AAV capsid, particle, impurity, or aggregate capable of binding an ion exchange chromatography column regardless of the source or serotype of the capsid. Because the methods of the present disclosure are applicable to any AAV or rAAV capsid capable of binding an ion exchange chromatography column, the capsid can be from any source, e.g., human, avian, bovine, canine, equine, primate, non-primate, ovine, or any derivation thereof.

In some embodiments, rAAV particles are derived from one or more AAVs selected from the group consisting of AAV1, AAV2, AAV3, AAV4, AAV5, AAV6, AAV7, AAV8, AAV9, AAV9.47, AAV9(hu14), AAV10, AAV11, AAV12, Rh8, Rh10, Rh74, AAV3B, AAV-218, LK03, RHM4-1, DJ, DJ8, NP59, Anc-80 and variants thereof, including the variants of AAV capsids set forth in Pulicherla et al., Mol. Ther., 19(6) 1070-1078 (2011) (describing AAV9) variants, including AAV9.47 among others). U.S. Pat. No. 7,906,111 (describing AAV9(hu14) among others), U.S. Pat. No. 10,532,111 (describing NP59 among others), U.S. Pat. No. 10,738,087 (describing Anc-80 among others), U.S. Pat. No. 9,169,299 (describing “LK03”), U.S. Pat. No. 9,840,719 (describing “RHM4-1”), U.S. Pat. Nos. 7,749,492, 7,588,772 (describing “DJ” and “DJ8”), U.S. Pat. No. 9,587,282, and patent applications WO2012/145601, WO2013/158879, WO2015/013313, WO2018/156654, US2013/0059732, all of which are incorporated herein by reference in their entireties, including an AAV capsid with peptide modifications, such as a cell targeting peptide.

In some embodiments, the full rAAV particles comprise a transgene that encodes a gene product selected from the group consisting of insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor a (TGFa), platelet-derived growth factor (PDGF), insulin growth factors I and II (IGF-I and IGF-II). TGFp, activins, inhibins, bone morphogenic protein (BMP), nerve growth factor (NGF), brain-derived neurotrophic factor (BDNF), neurotrophins NT-3 and NT4/5, ciliary neurotrophic factor (CNTF), glial cell line derived neurotrophic factor (GDNF), neurturin, agrin, netrin-1 and netrin-2, hepatocyte growth factor (HGF), ephrins, noggin, sonic hedgehog and tyrosine hydroxylase.

In some embodiments, the full rAAV particles comprise a transgene that encodes a gene product selected from the group consisting of thrombopoietin (TPO), interleukins (ILI through IL-17), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand, IgG, IgM, IgA, IgD and IgE, chimeric immunoglobulins, humanized antibodies, single chain antibodies, T cell receptors, chimeric T cell receptors, single chain T cell receptors, class I and class II MHC molecules.

In some embodiments, the full rAAV particles comprise a transgene that encodes a protein useful for correction of in born errors of metabolism selected from the group consisting of carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, factor V, factor VIII, factor IX, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, RPE65, H-protein, T-protein, a cystic fibrosis transmembrane regulator (CFTR) sequence, and a dystrophin cDNA sequence.

In some embodiments, the full rAAV particles comprise a transgene that encodes Factor VIII and Factor IX.

Full rAAV particles of interest to be purified by a process according to an embodiment of the application can be produced by host cells. As an initial step, typically host cells that produce the rAAV virions can be harvested, optionally in combination with harvesting cell culture supernatant (medium) in which the host cells (suspension or adherent) producing rAAV virions have been cultured. In methods herein, the harvested cells and optionally cell culture supernatant can be used as is, as appropriate, or concentrated. Further, if infection is employed to express accessory functions, residual helper virus can be inactivated. For example, adenovirus can be inactivated by heating to temperatures of approximately 60° C. for, e.g., 20 minutes or more, which inactivates only the helper virus since AAV is heat stable while the helper adenovirus is heat labile.

Cells and/or supernatant of the harvest are lysed by disrupting the cells, for example, by chemical or physical means, such as detergent, microfluidization and/or homogenization, to release the rAAV particles. Concurrently during cell lysis or subsequently after cell lysis, a nuclease such as benzonase can be added to degrade contaminating DNA. Typically, the resulting lysate is clarified to remove cell debris, such as filtering, centrifuging, to render a clarified cell lysate. In a particular example, lysate is filtered with a micron diameter pore size filter (such as a 0.1-10.0μιη pore size filter, for example, a 0.45μιη and/or pore size 0.2μηι filter), to produce a clarified lysate.

The lysate (optionally clarified) contains AAV particles (full rAAV particles and AAV non-full particles) and AAV vector production/process related impurities, such as soluble cellular components from the host cells that can include, inter alia, cellular proteins, lipids, and/or nucleic acids, and cell culture medium components. The optionally clarified lysate is then subjected to additional purification steps to purify full AAV particles from impurities using chromatography. Clarified lysate may be diluted or concentrated with an appropriate buffer prior to the chromatography method of the application.

### EXAMPLES

### Example 1. Resin Screening for Separation of Full rAAV Particles and Non-full Particles

In this Example, different resins were screened to identify the best resin for the separation of full rAAV particles and non-full particles.

### Method and Material

The sample contained non-full particles and full RHM4-1 rAAV particles at a ratio of 2.4:1 (i.e., 70% impurity/30% product). The column size is 0.353 mL 3 mm×50 mm. The resin screening was conducted using pulse loading (10% of binding capacity) and a residence time of 1 minute. During the screening, 3 pH values (8.0, 8.6, and 9.25) and three binding strengths with salt (6.8, 5, and 1.6 mS/cm) were tested. The loading buffer was 50 mM Tris or 25 mM Tris. The flowthrough effluent from the column was analyzed by high performance liquid chromatography (HPLC) to determine the separation of the full and non-full particles.

### Results

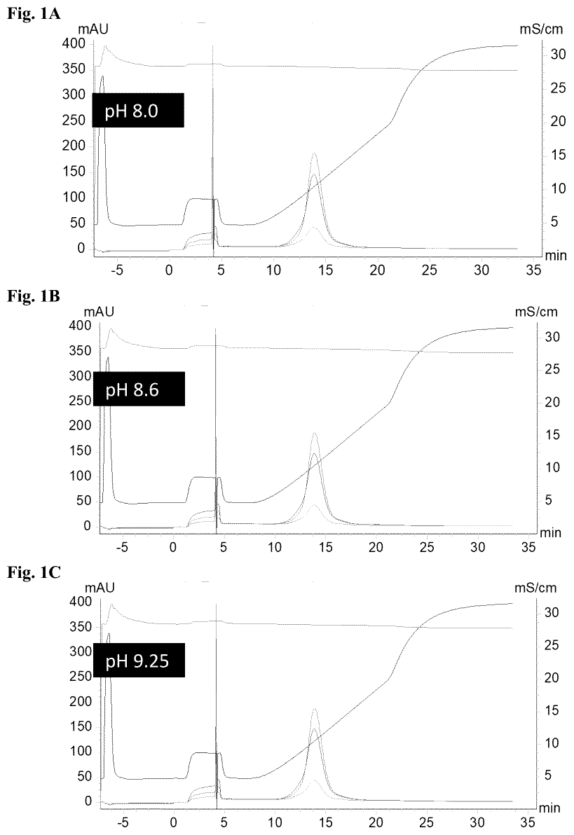

Poros 50 HQ Resin (Thermo Scientific; POROS™—(Waltham, MA)): This resin is based on quaternized polyethyleneimine functional group some weak anion exchange (AEX) functionality. As indicated in FIGS. 1A-C, the separation of full and non-full particles on Poros 50 HQ resin was not significantly different at all three pH values under 50 mM Tris and 30 mM NaCl, while the separation at pH 8.6 was only slightly better compared to the other two pH values.

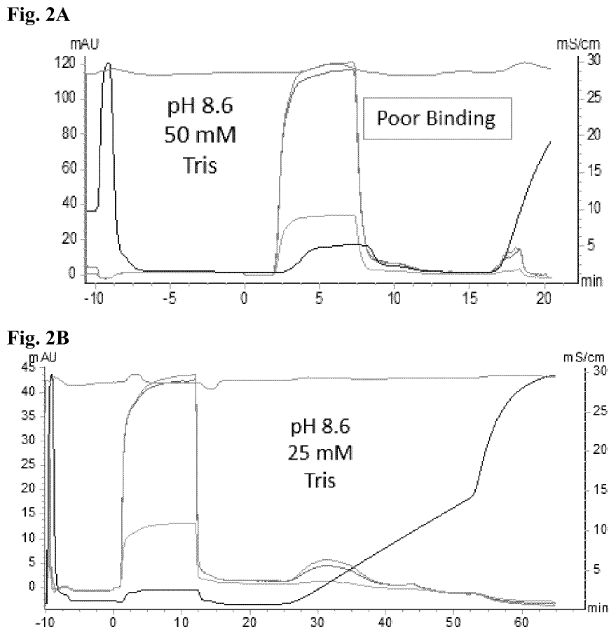

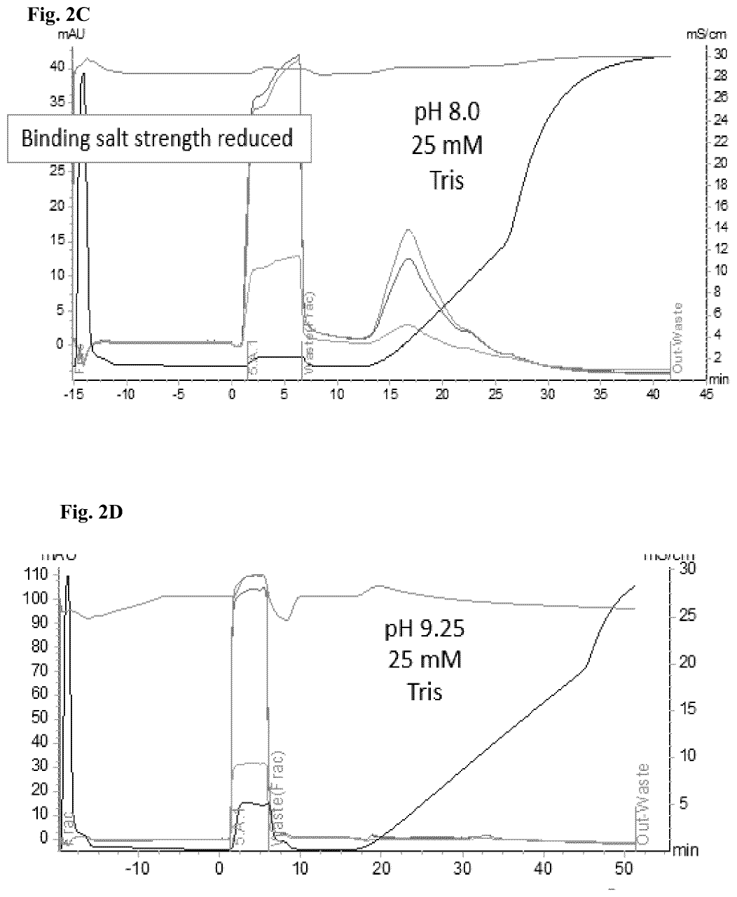

Poros 50 D Resin: This resin is based on dimethylaminopropyl functional group. As indicated in FIGS. 2A-D, the binding of rAAV particles to Poros 50 D resin was poor under the screening conditions, and decreased with increased pH. Various loading buffers were used in the figures: pH 8.6 and 50 mM Tris (FIG. 2A), pH 8.6 and 25 mM Tris (FIG. 2B), pH 8.0 and 25 mM Tris (FIG. 2C), and pH 9.25 and 25 mM Tris (FIG. 2D).

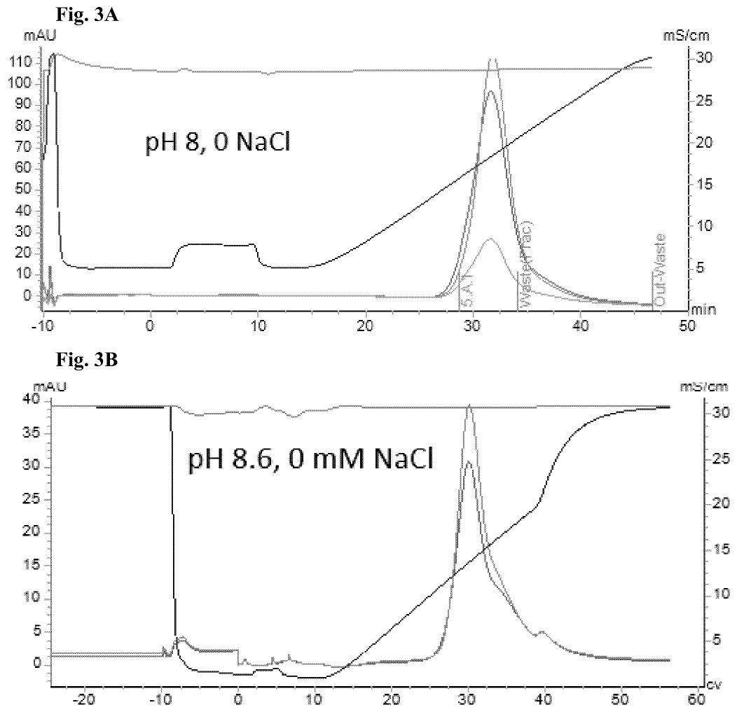

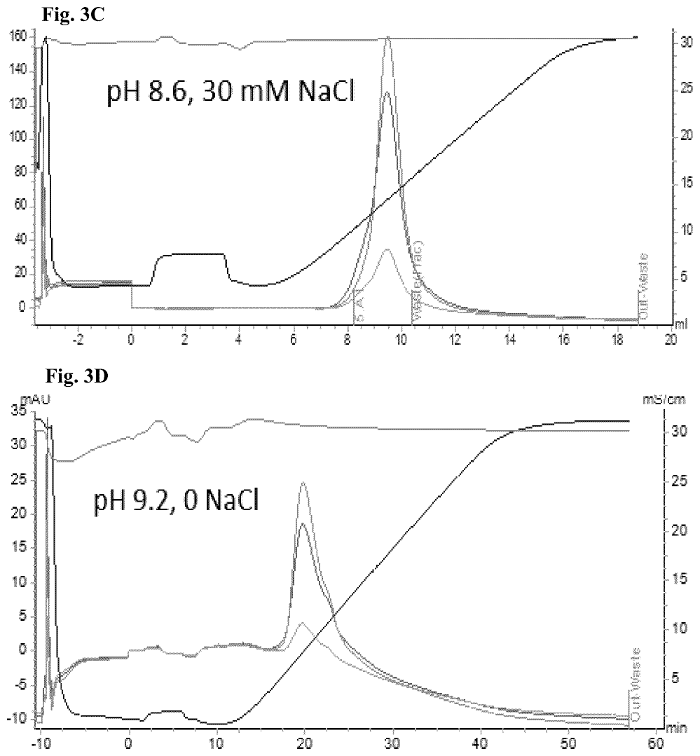

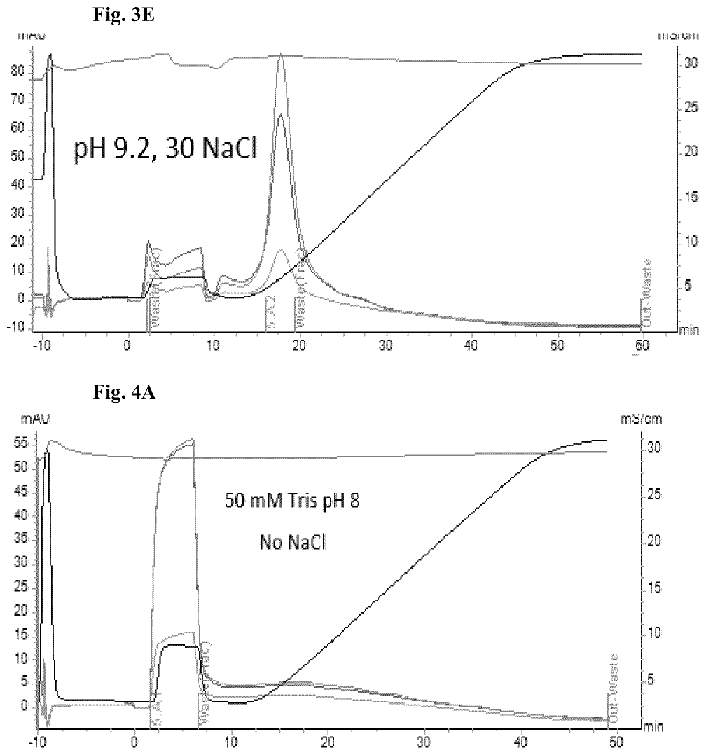

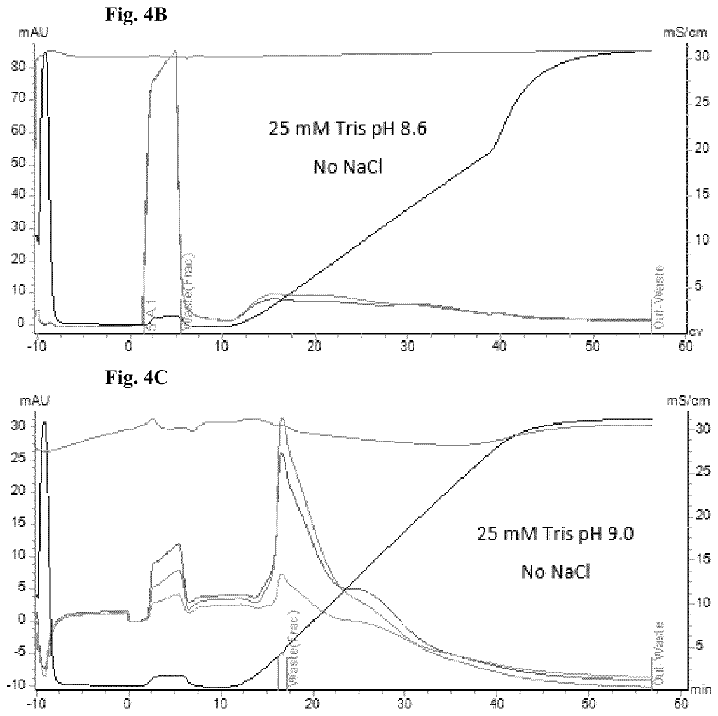

Poros 50 PI Resin: This resin is based on polyethyleneimine functional group. As indicated in FIGS. 3A-E, the binding of rAAV particles to Poros 50 PI resin decreased with increased pH. It was also noted that addition of NaCl only slightly improved the resolution. The figures evaluate Poros 50 PI resin at different pH and salt concentrations: pH 8.0 without NaCl (FIG. 3A), pH 8.6 without NaCl (FIG. 3B), pH 8.6 and 30 mM NaCl (FIG. 3C), pH 9.2 without NaCl (FIG. 4C), and pH 9.2 and 30 mM NaCl (FIG. 3E).

Capto ImpRes Q Resin: This resin employs Capto™ (Cytiva Life Sciences—Marlborough, MA) base agarose matrix with ionic group ligands. As indicated in FIGS. 4A-C, the binding of rAAV particles to Capto ImpRes Q resin increased with increased pH. However, the separation of full and non-full particles was poor on Capto ImpRes Q Resin under the screening conditions. The figures evaluate Capto ImpRes Q resin at different pH values with different buffers: pH 8.0 and 50 nm Tris without NaCl (FIG. 4A), pH 8.6 and 25 mM Tris without NaCl (FIG. 4B), and pH 9.0 and 30 mM Tris without NaCl (FIG. 4C).

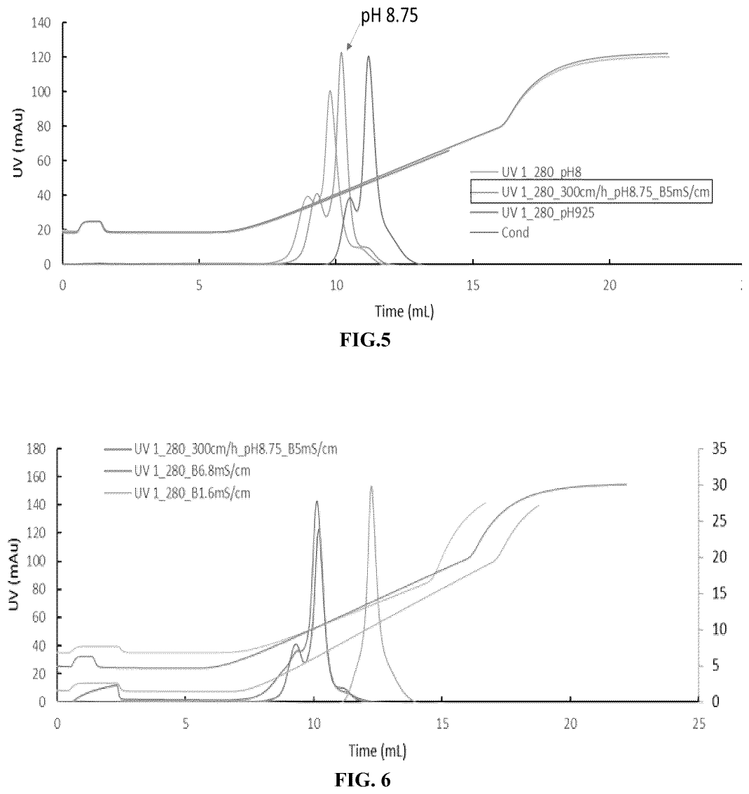

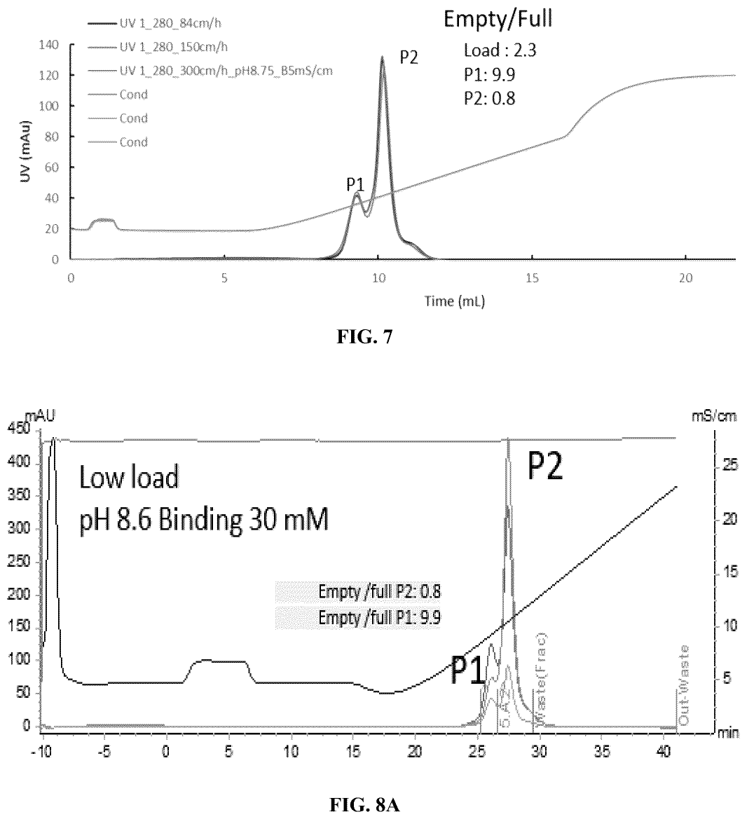

Poros XO Resin: This resin is based on proprietary quaternary amine functional group. The screening demonstrated that Poros XQ resin was best for the separation of full and non-full particles. As indicated in FIG. 5, the separation was best at pH 8.75 compared to other pH values (pH 8 and 9.25). The binding salt also affected the separation, as indicated in FIG. 6. In addition, the separation was similar under different flow rates (84 cm/h, 150 cm/h, and 300 cm/h) when conducted at pH 8.75, as indicated in FIG. 7.

### Example 2. Optimization of Displacement Chromatography with Poros XQ Resin

In this Example, different conditions were screened to identify optimal conditions for displacement chromatography separation of full RHM4-1 rAAV particles and non-full particles on Poros XQ resin.

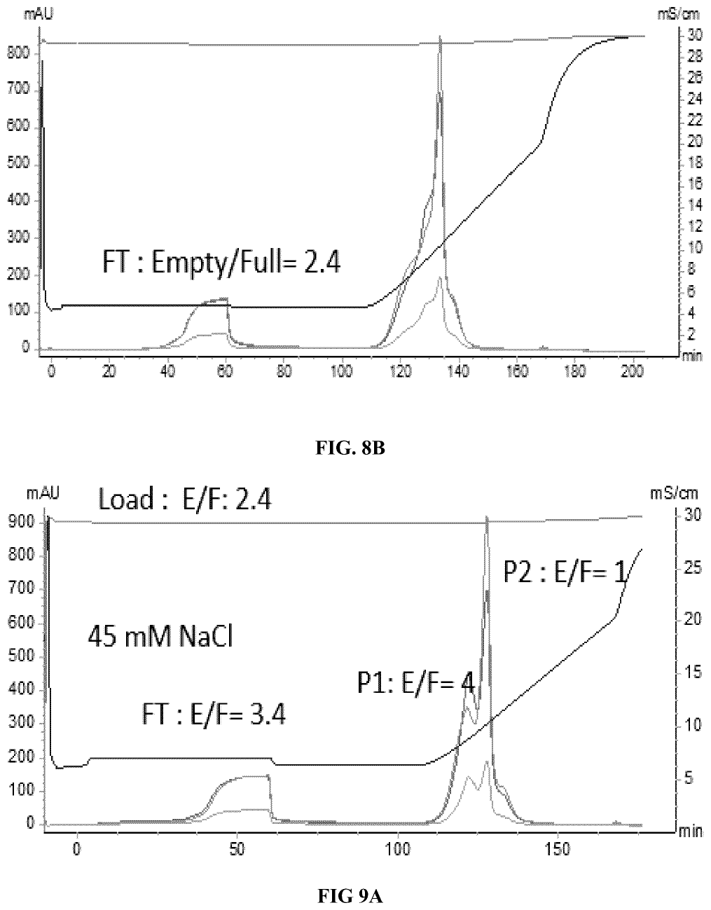

FIG. 8A demonstrates that there were two product peaks in the HPLC analysis of the effluent when the sample was loaded in 30 mM Tris at pH 8.6 and the loading amount was low. Among the two peaks, the ratio of non-full particles to full particles (E/F) was 9.9 in peak P1, while the ratio of E/F in peak P2 was 0.8. This indicates that the affinity of non-full particles binding to Poros XQ resins is weaker than the affinity of full particles binding to the resins, and thus the non-full particles eluted earlier than the full particle.

In contrast, when the sample was loaded in an amount exceeding the binding capacity, the ratio of E/F in the flowthrough (FT) was 2.4 in the breakthrough, as indicated in FIG. 8B. FIG. 8B demonstrates that the addition of 30 mM NaCl into the load was not sufficient to promote displacement. Therefore, the flowthrough's E/F ratio is the same as that of the load.

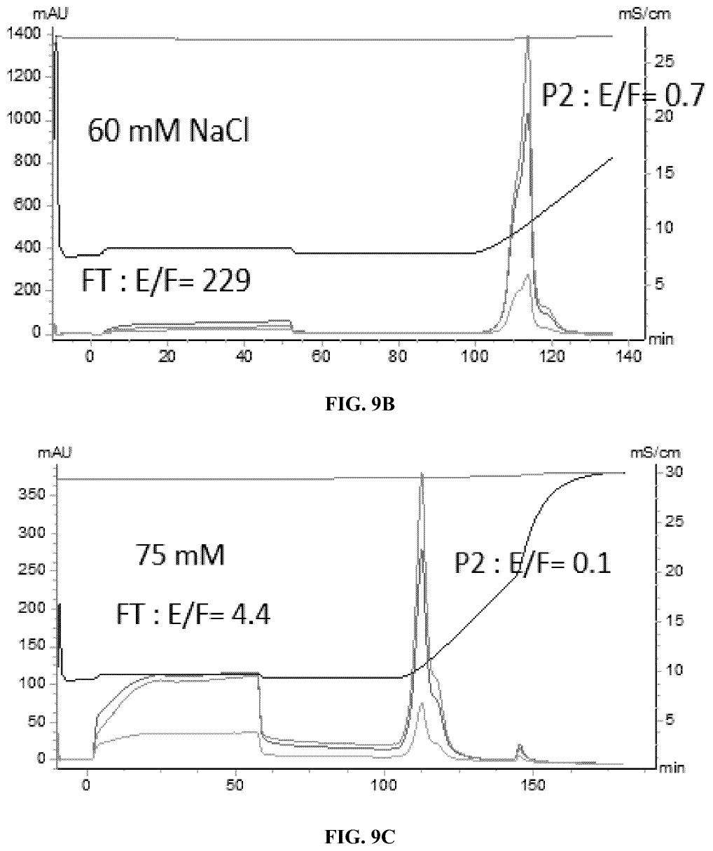

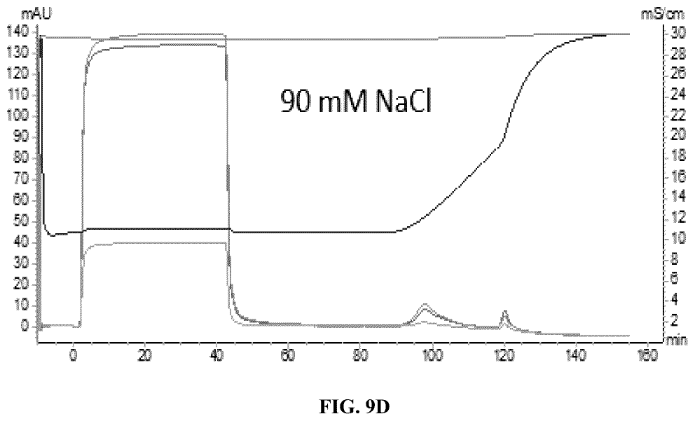

Next, different binding strengths at different salt concentrations were screened to identify the optimal salt concentration for better separation of non-full and full particles under the displacement column chromatography. As shown in FIGS. 9A and 9B, increasing the NaCl concentration in the loading buffer from 45 mM to 60 mM, respectively, increased the ratio of E/F in the flowthrough from 3.4 to 229, indicating a greater displacement of the non-full particles by the full particles at the higher salt concentration. At the same time, the purity of product peak P2 was improved from E/F=1 to E/F=0.7.

However, increasing the salt concentration further to 75 mM and 90 mM did not further enhance the displacement, as the overall affinity of the sample for the resin was reduced, as shown in FIGS. 9C and 9D, respectively. Therefore, FIGS. 9A-D indicates that optimal displacement occurred at an intermediate binding strength, where the difference in the binding affinities of the non-full and full particles was most pronounced. More generally, the results indicate that the binding strength adjustable with the salt concentration can be used as a handle to promote displacement of the non-full particles, which can result in improved resolution.

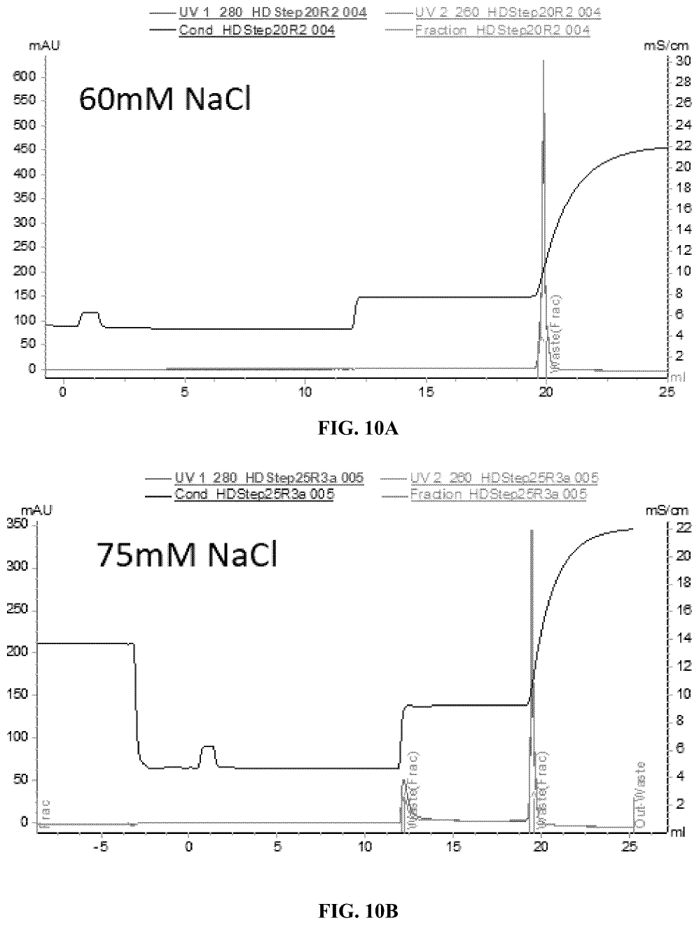

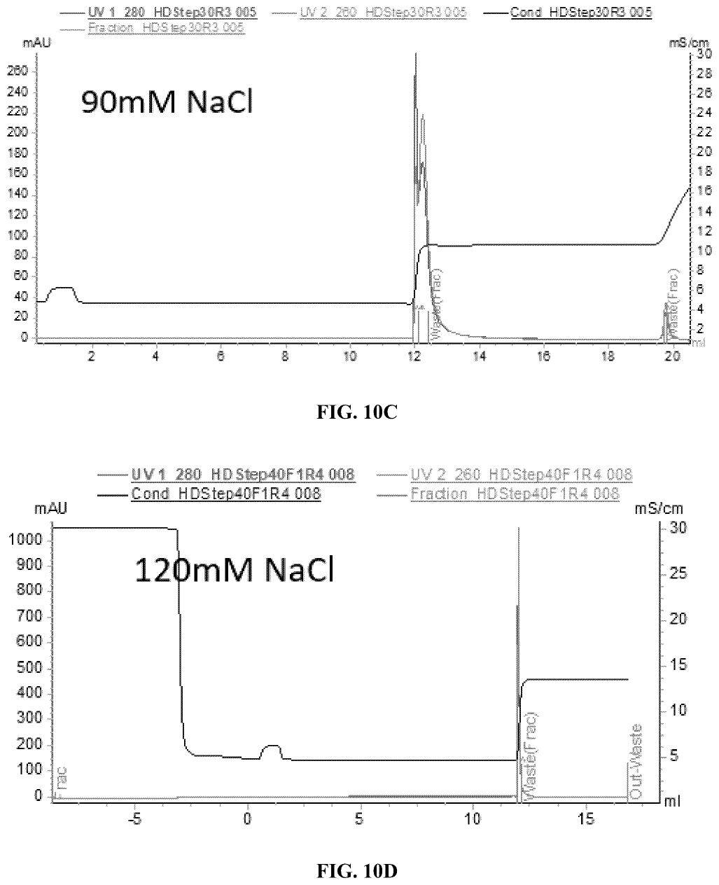

In addition, step elution conditions were also tested to determine the salt range in the elution buffer. The loading buffer used for the tests contained 50 mM Tris pH 8.5 and 30 mM NaCl. As shown in FIGS. 10A-D, the step elution worked for all tested elution buffers with NaCl at a concentration of: 60 mM, 75 mM, 90 mM, and 120 mM, respectively.

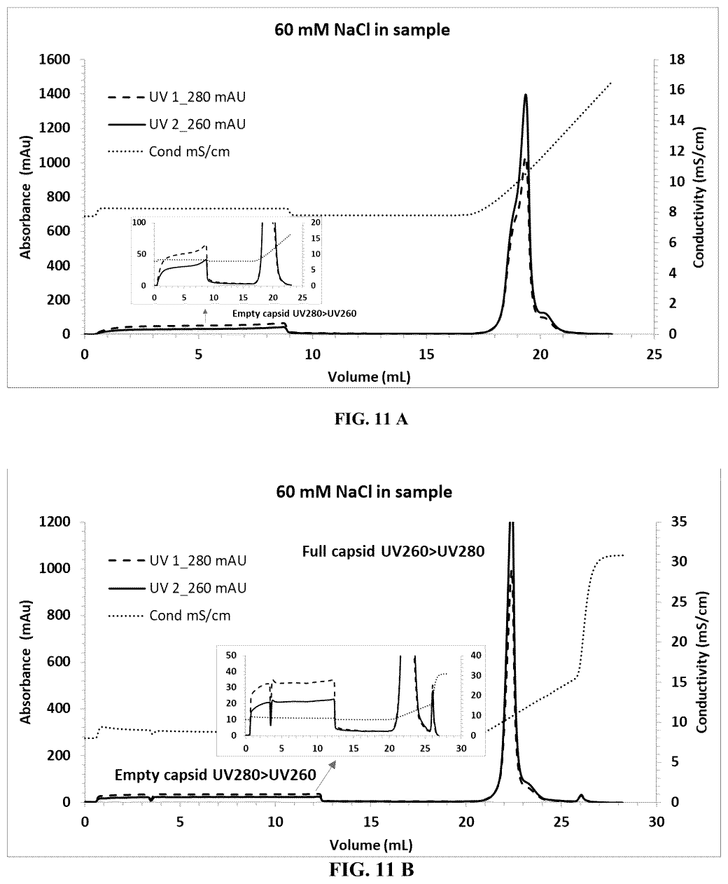

Further displacement was also studied in this Example. Further displacement occurred when more sample was loaded exceeding the breakthrough load, as shown in FIGS. 11A-B, where the non-full particles bound to the bottom part of the column were also displaced by the full particles and exited in the flowthrough. FIG. 11B demonstrated that the purity of rAAV in the purified preparation was improved to nearly 100% due to the further displacement. In addition, the further displacement chromatography also afforded the purified full particles at high yield, which was greater than 90%.

### Example 3. Use of CaCl₂) as Additive in Sample Load and Wash Buffer

In the study of separation of full RHM4-1 rAAV particles and non-full particles with Poros XQ Resin, the product loss was noticed in the displacement chromatography purification. For example, in the breakthrough run, when the loading buffer was 50 mM Tris/60 mM NaCl at pH 8.5, the recovery yield was 89.81% with single column process and 74.92% for the proposed two-column process. It appears that the loss mainly resulted from the presence of full particles in the flowthrough. The addition of additives in the sample load or wash buffer was studied to improve resolution and decrease the amount of full rAAV particles in the flowthrough, thus increasing the recovery yield.

The addition of MgCl₂ in the sample load was first tested as shown in FIG. 12. MgCl₂ could be used as an additive to improve resolution. However, in the subsequent wash step, the concentration of MgCl₂ needed to be increased to wash off the non-full particles bound to the column. It was found that the addition of MgCl₂ in the sample load is not robust even under non-binding conditions, because the difference in conductivity between the load and elution buffer was only about 1 mS/cm.

Other additives were also tested. CaCl₂) was identified as an additive that resulted in the best flowthough for the purification on Poros XQ Resin. As shown in FIG. 13, the addition of CaCl₂) improved the separation of non-full and full rAAV particles. In addition, with the use of CaCl₂), the difference in conductivity between the load and elution buffer was robust, e.g., at about 4 mS/cm.

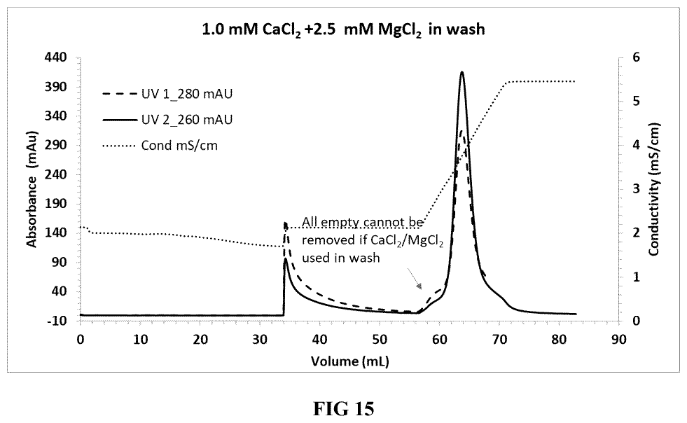

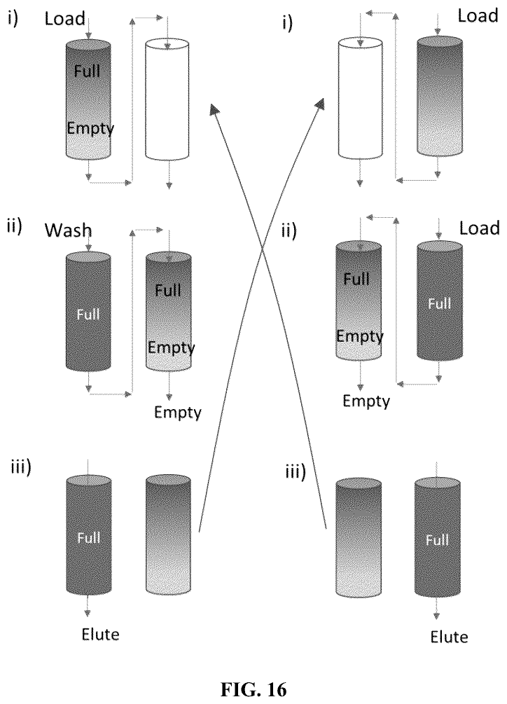

It was also found that, if CaCl₂) was not added in the sample load, both the yield and the product purity were compromised in the purification of full rAAV particles from the non-full particles. As shown in FIG. 15, when no additive other than NaCl was used in the sample load and the wash buffer contained 1 mM CaCl₂) and 2.5 mM MgCl₂, it was difficult to remove all non-full particles bound to the column via wash. Furthermore, during the subsequent elution with increased CaCl₂) concentration (FIG. 15), the bound non-full particles appeared to be instable on the resin and generated new impurity which in turn affected the product purity.

Therefore, the inventors surprisingly found adding CaCl₂) in the sample load, preferably also the wash buffer and elution buffer, resulted in optimal separation and improved yield.

### Example 4. Use of LiCl as Additive in Sample Load and Wash Buffer

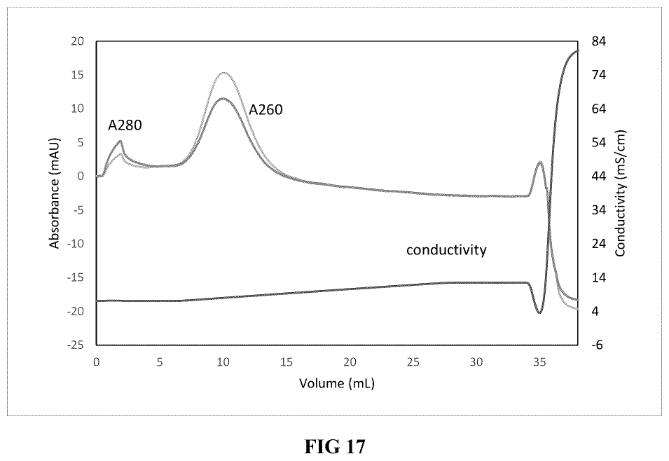

Lithium chloride was another additive tested for purification on Poros XQ Resin. As shown in FIG. 17, the addition of LiCl improved the separation of non-full and full RHM4-1 rAAV particles. In addition, with the use of LiCl, the difference in conductivity between the load and elution buffer was at about 0.2 mS/cm.

### Example 5. Enhanced Improvement in Separation and Yield in Displacement Chromatography with CaCl₂) Added to the Sample Load and Wash Buffer

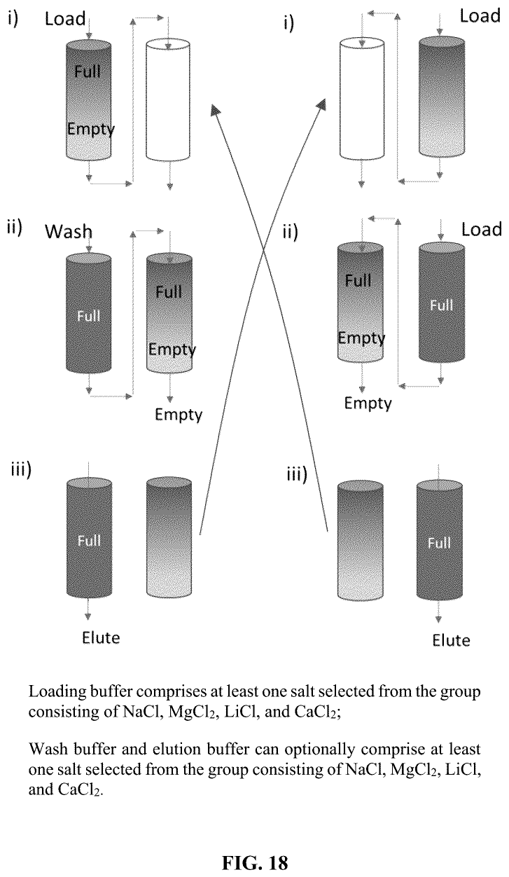

The improved separation of non-full and full RHM4-1 rAAV particles from the use of additives, like CaCl₂), in the loading buffer, and optionally in the wash and elution buffers, shown in Examples 3 and 4 can also be used in conjunction with the displacement chromatography method shown in Example 2. Combined, as shown in FIG. 18, these methods will yield optimal separation and yield of full rAAV particles over non-full particles.

### Example 6. Enhanced Improvement in Separation with Additives and Decreasing pH

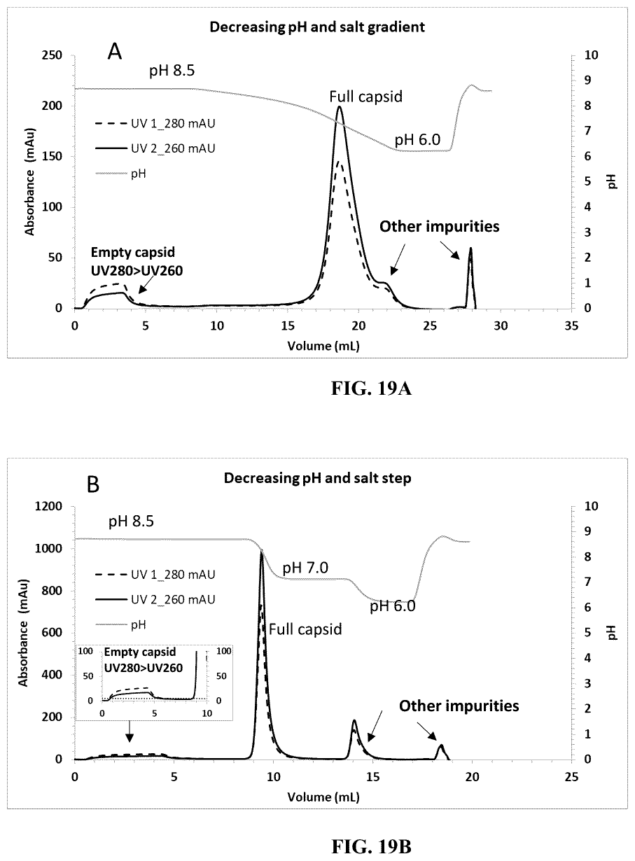

The improved separation of non-full and full RHM4-1 rAAV particles from the use of additives, like CaCl₂), in the loading buffer, and optionally in the wash and elution buffers, shown in Examples 3 and 4, can be enhanced by decreasing pH with gradient or step elution as shown in FIG. 19.

### Example 7. Enhanced Improvement in Separation with Additive (NH₄)₂SO₄

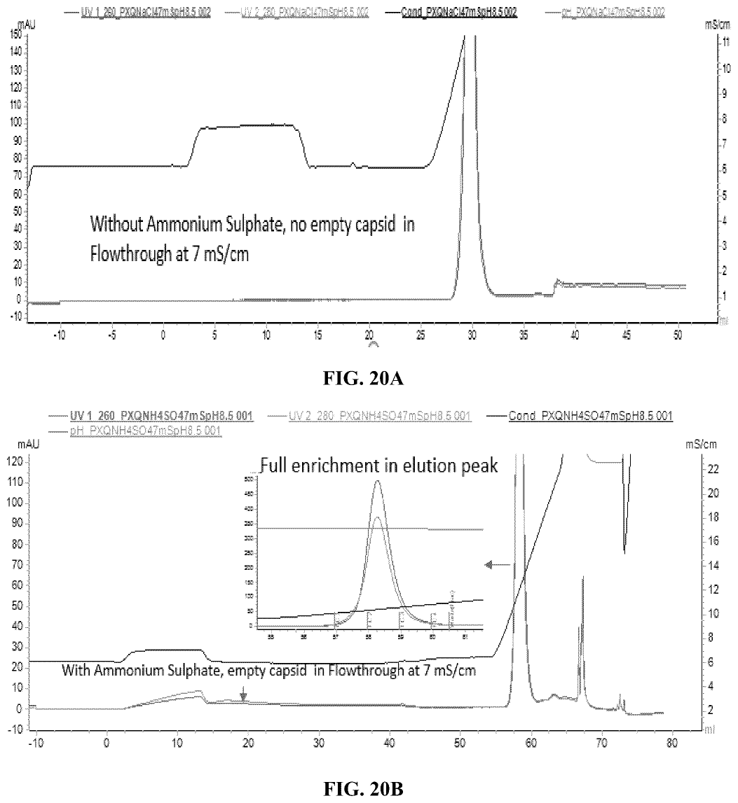

The improved separation of empty and full RHM4-1 rAAV particles on PXQ resin can be achieved by the use of (NH₄)₂SO₄ as an additive in the loading buffer as shown in FIGS. 20A-B.

### Example 8. Enhanced Improvement in Separation with Additive (NH₄)₂SO₄

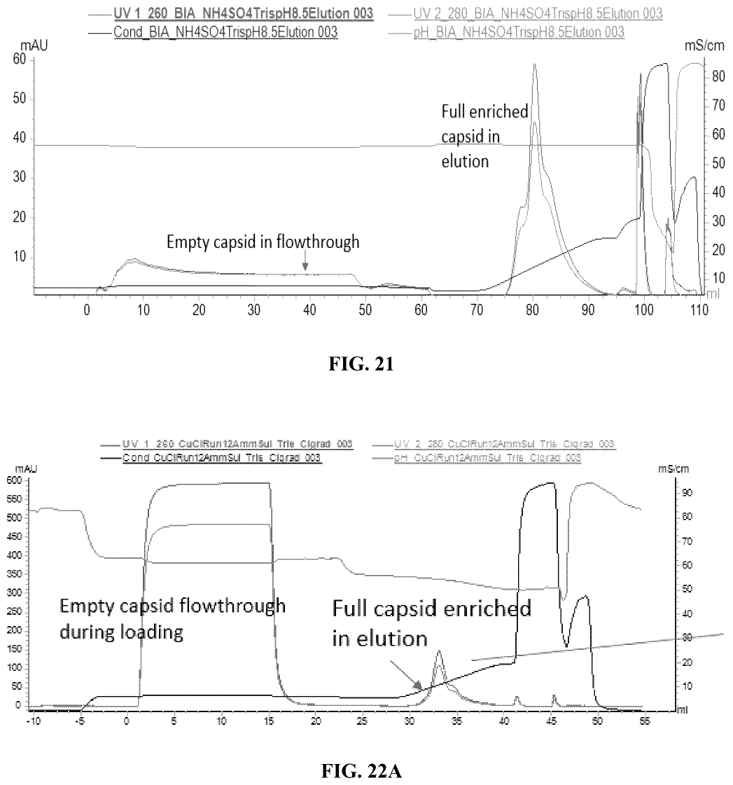

The improved separation of empty and full RHM4-1 rAAV particles on BIA monolith resin can be achieved by the use of (NH₄)₂SO₄ as an additive in the loading buffer as shown in FIG. 21.

### Example 9. Enhanced Improvement in Separation with Additive CuCl₂

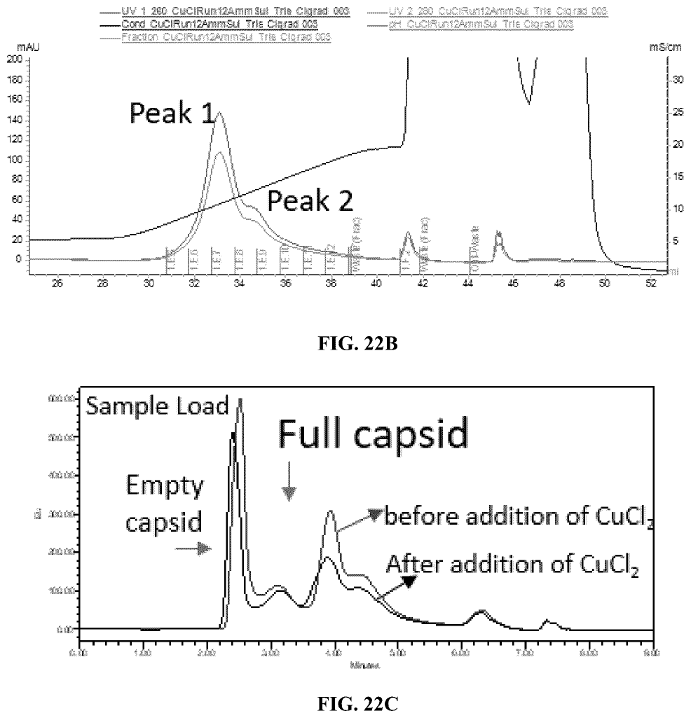

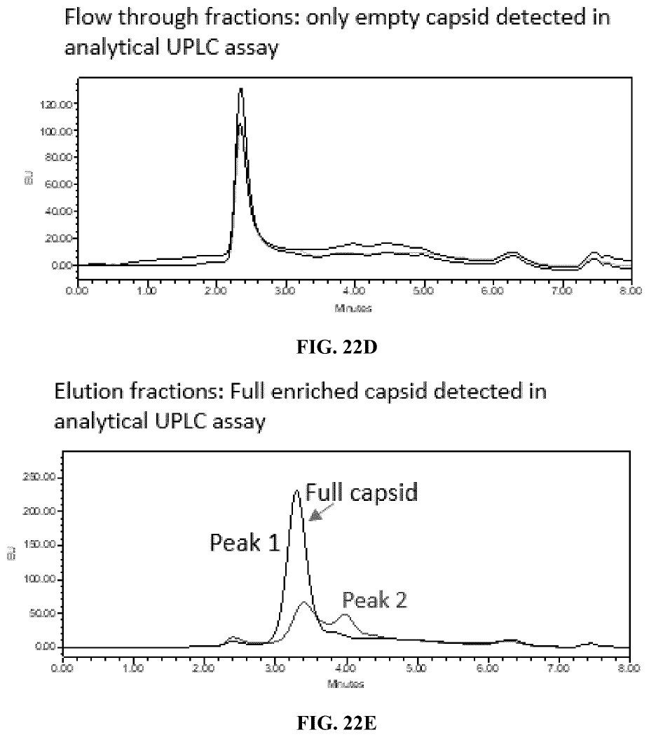

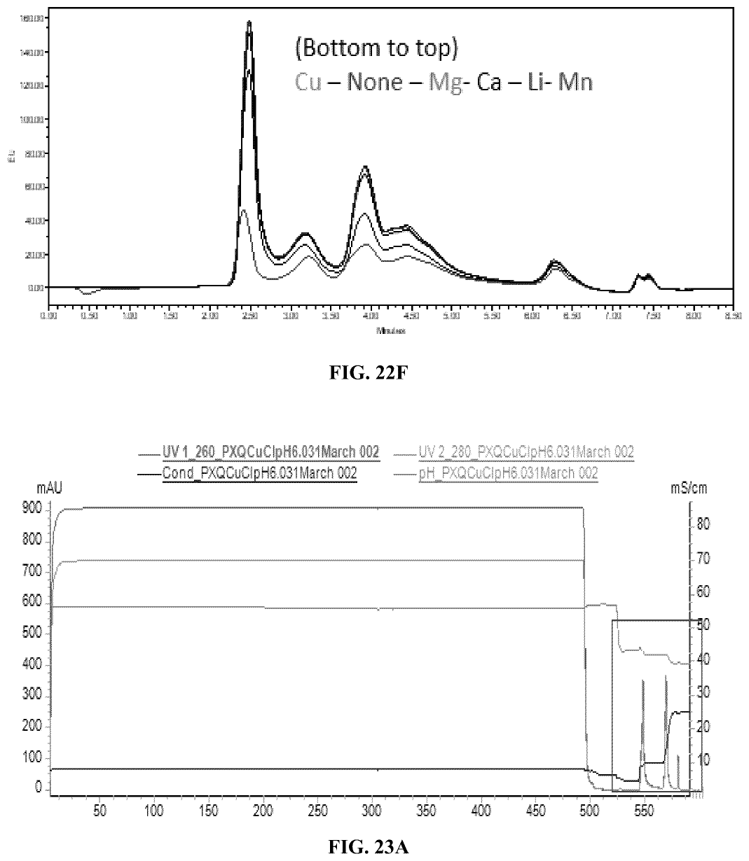

The improved separation of empty and full RHM4-1 rAAV particles on PXQ resin can be achieved by the use of CuCl₂ as an additive in the loading buffer as shown in FIGS. 22 A-E.

### Example 10

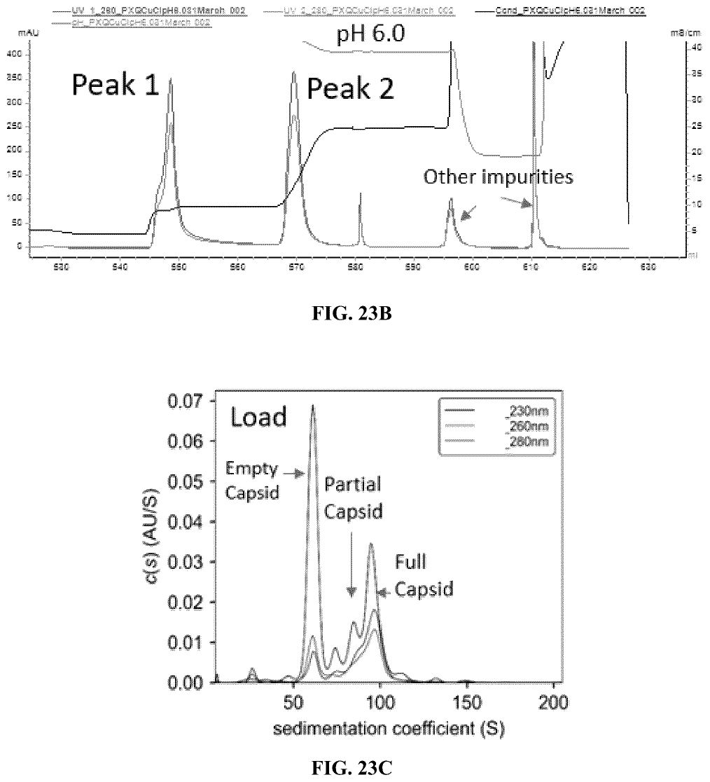

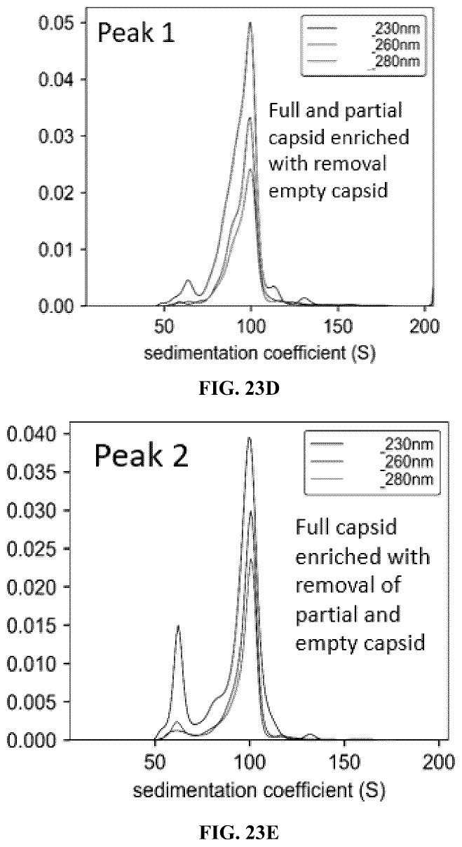

Enhanced Improvement in Separation of Partial Particles with CuCl₂ and Elution with Lower pH and Increasing Salt. The improved separation of empty, partial and full LK03 rAAV particles from the use of additives, like CuCl₂, in the loading buffer, can be enhanced by elution at lower pH with gradient or step elution with increasing salt as shown in FIGS. 23A-E.

### Example 11

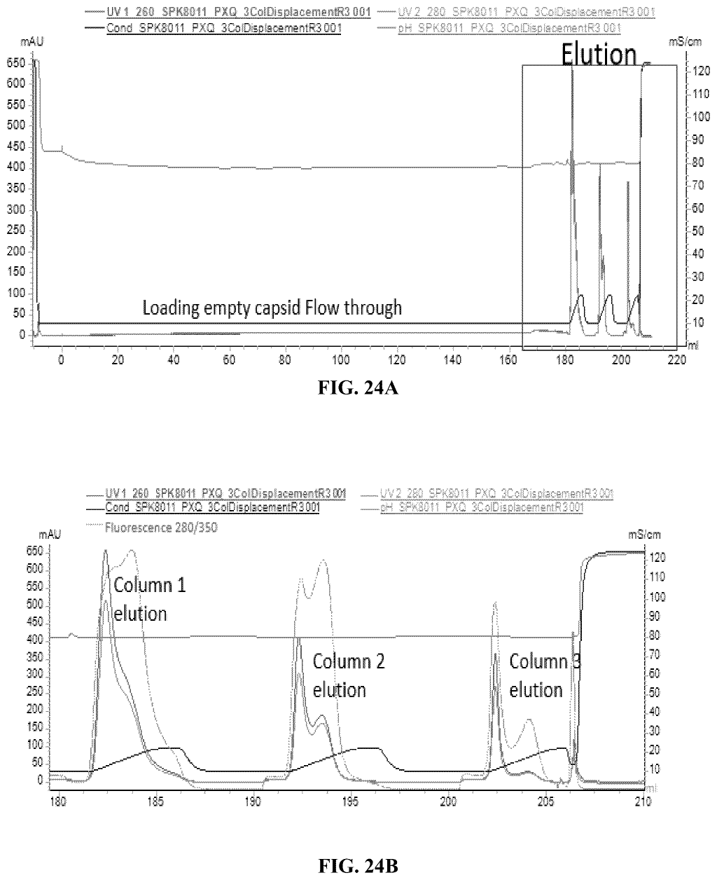

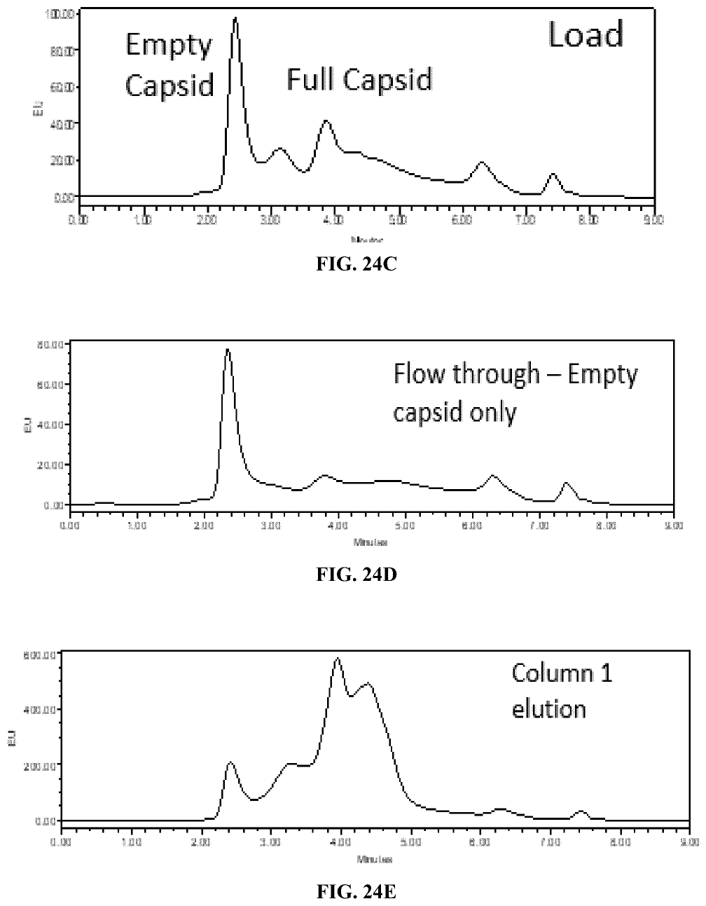

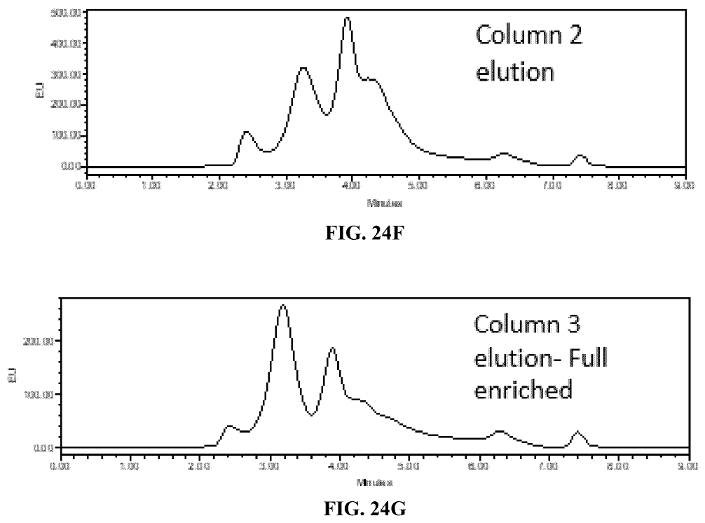

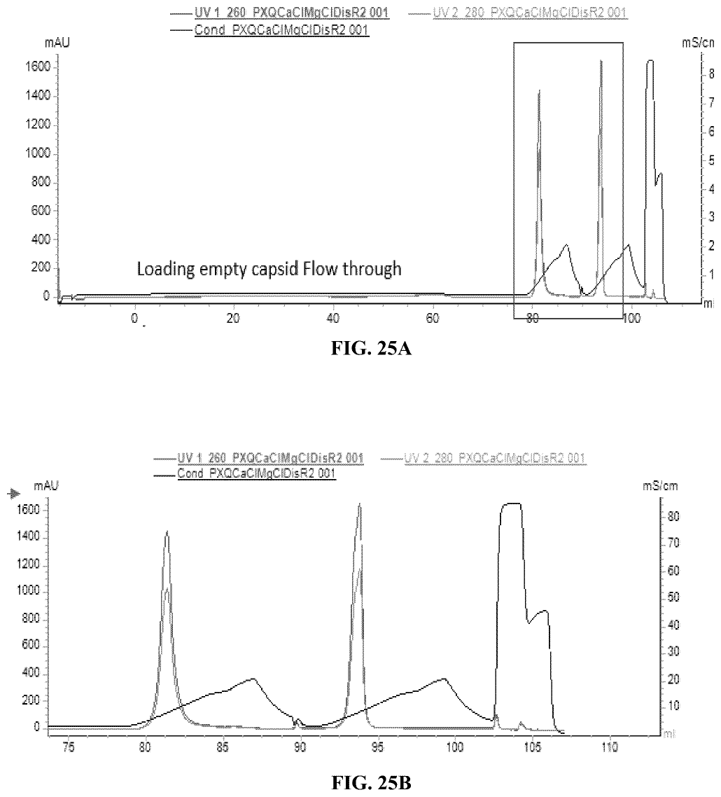

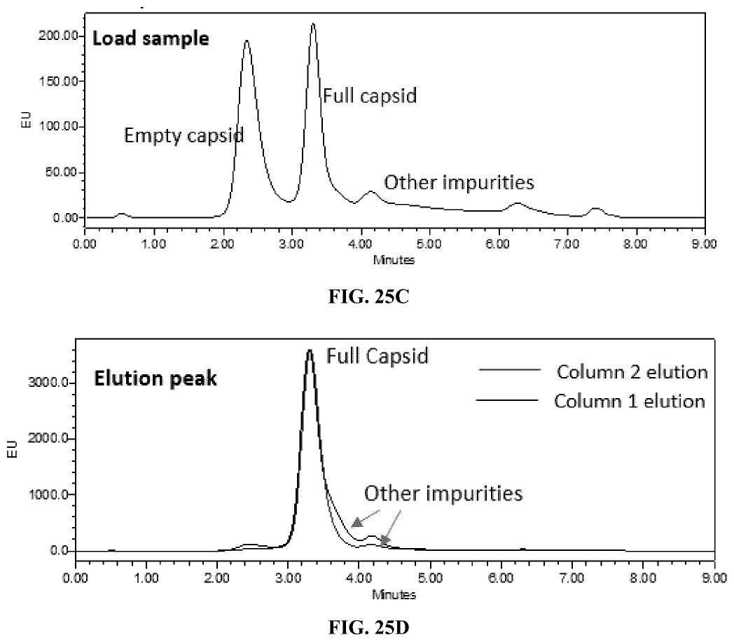

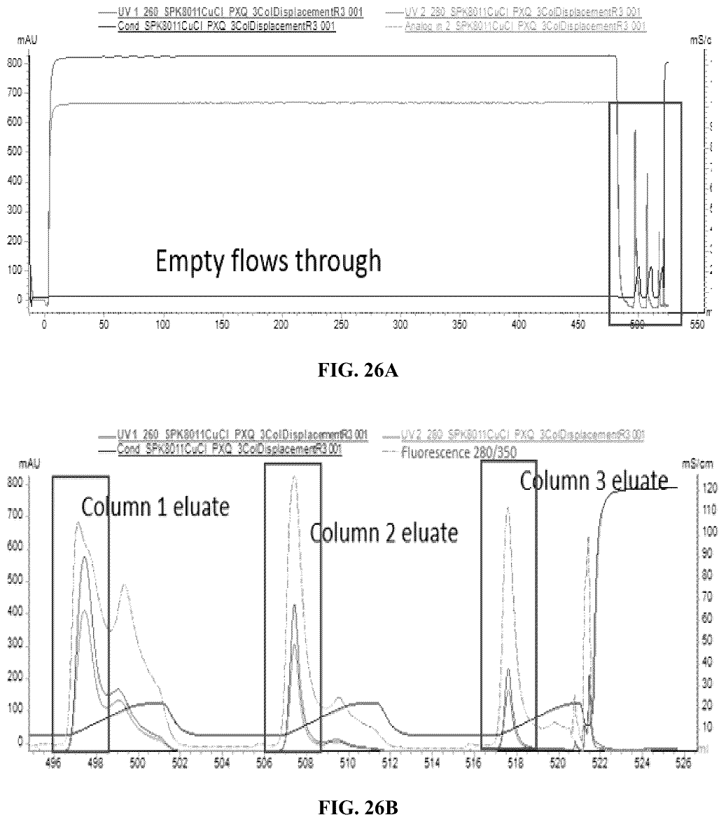

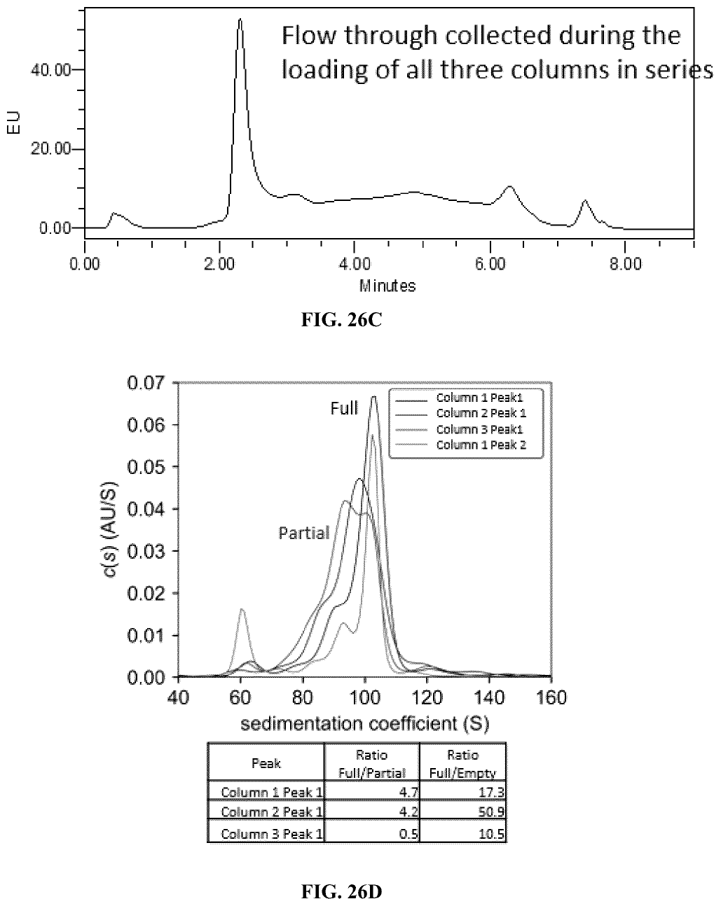

Enhanced Improvement in Separation of Partial Particles with Multiple Columns. The improved separation of empty, partial and full LK03 rAAV particles observed using 2 columns can be enhanced by using at least 3 columns, as shown in FIGS. 24-26. The method can be used to remove impurities and aggregates that have a stronger affinity for the column than the full particles, as shown in FIG. 25. In this two column set up, the second column is enriched with full particles as the aggregates and impurities that have a stronger affinity for the column than the full particles have are bound to the first column. In a multiple column setup, the first column is enriched in the strongest binding particle (highest retention time) while subsequent columns are enriched in the next strongest binding particles. Upon determining the column for which the target rAAV particle or impurity has greatest affinity, and one can employ the disclosed method to purify the desired rAAV particle or impurity. This example further demonstrates the use of the disclosed method for the enrichment of different particle variants on a multiple column setup.

It is understood that the examples and embodiments described herein are for illustrative purposes only, and that changes could be made to the embodiments described above without departing from the broad inventive concept thereof. For example, the inventive concept includes separation of partial particles from empty particles, or as shown in FIG. 25, separating impurities that have a stronger affinity for the resin than full particles, to enrich a second column with full particles as opposed to the first column. It is understood, therefore, that this invention is not limited to the particular embodiments disclosed, but it is intended to cover modifications within the spirit and scope of the invention as defined by the appended claims.

1. A method for purifying full recombinant adeno-associated virus (rAAV) particles comprising:
(a). providing an rAAV preparation comprising full rAAV particles and non-full rAAV particles;
(b). loading the rAAV preparation in a loading buffer to a column comprising a chromatography medium, wherein the full rAAV particles have a higher binding affinity to the chromatography medium than the non-full particles, and the quantity of the full rAAV particles and non-full particles applied to the column exceeds the binding capacity of the chromatography medium, such that the non-full particles bound to the chromatography medium are displaced by the full rAAV particles into the flowthrough from the column; and
(c). eluting the full rAAV particles bound to the chromatography medium with an elution buffer to obtain a purified preparation.

2. The method according to claim 1, wherein the chromatography medium is an ion exchange column chromatography medium, preferably an anion exchange chromatography medium.

3. The method according to claim 2, wherein the column chromatography medium is selected from the group consisting of Poros 50 HQ, Poros 50 D, Poros 50 PI, Capto ImpRes Q, CIMmultus™ QA Monolithic Column and Poros XQ, preferably Poros XQ.

4. A method for purifying full recombinant adeno-associated virus (rAAV) particles comprising:
(a). providing an rAAV preparation comprising the full rAAV particles and non-full particles;
(b). loading a first batch of the rAAV preparation in a loading buffer to a first column comprising a first chromatography medium, wherein the full rAAV particles have a higher binding affinity to the first chromatography medium than the non-full particles, and the quantity of the full rAAV particles and non-full particles applied to the first column exceeds the binding capacity of the first chromatography medium such that the non-full particles bound to the first chromatography medium are displaced by the full rAAV particles into a first load flowthrough from the first column;
(c). loading the first load flowthrough to a second column comprising a second chromatography medium to obtain a loaded or partially loaded second column, wherein the second chromatography medium is the same type as the first chromatography medium;
(d). optionally, washing the first column with a wash buffer to obtain a washed first column;
(e). eluting the full rAAV particles bound to the first chromatography medium with an elution buffer to obtain a first eluate from the first column and an eluted first column, wherein the first eluate comprises an increased ratio of the full rAAV particles to the non-full rAAV particles;
(f). optionally, a second batch of the rAAV preparation in loading buffer is applied to the at least partially loaded second column, wherein the quantity of the full rAAV particles and non-full particles applied to the second column exceeds the binding capacity of the second chromatography medium such that the non-full particles bound to the second chromatography medium are displaced by the full rAAV particles into a second load flowthrough from the second column;
(g). optionally, loading the second load flowthrough from the second column to the eluted first column after step (e) to obtain a second loaded first column, and optionally washing second loaded first column before continuing to elute in step (e);
(h). optionally, washing the second column with a wash buffer to obtain a washed second column;
(i). eluting the full rAAV particles bound to the second chromatography medium with an elution buffer to obtain a second eluate and an eluted second column, wherein the second eluate comprises an increased ratio of the full rAAV particles to the non-full rAAV particles; and
(j). combining the first eluate and second eluate to produce a purified preparation of full rAAV particles.

5. The method according to claim 4, wherein the first chromatography medium and/or the second chromatography medium is an ion exchange column chromatography medium, preferably an anion exchange chromatography medium.

6. The method according to claim 4 or 5, wherein the steps (b) to (i) are performed with one or more cycles.

7. The method according to any one of claims 4-6, wherein the second column is partially loaded after step (c).

8. The method according to any one of claims 4-7, wherein the first column chromatography medium is selected from the group consisting of Poros 50 HQ, Poros 50 D, Poros 50 PI, Capto ImpRes Q, CIMmultus™ QA Monolithic Column and Poros XQ, preferably Poros XQ.

9. A method for purifying full recombinant adeno-associated virus (rAAV) particles comprising:
(a). providing an rAAV preparation comprising the full rAAV particles and non-full particles;
(b). loading a first batch of the rAAV preparation in a loading buffer to a first column comprising a first chromatography medium, wherein the full rAAV particles have a higher binding affinity to the first chromatography medium than the non-full particles, and the quantity of the full rAAV particles and non-full particles applied to the first column exceeds the binding capacity of the first chromatography medium such that the non-full particles bound to the first chromatography medium are displaced by the full rAAV particles into a first load flowthrough from the first column;
(c). loading the first load flowthrough to a second column comprising a second chromatography medium to obtain a loaded or partially loaded second column, wherein the second chromatography medium is the same type as the first chromatography medium;
(d). optionally, washing the first column with a wash buffer to obtain a washed first column;
(e). eluting the full rAAV particles bound to the first chromatography medium with an elution buffer to obtain a first eluate from the first column and an eluted first column, wherein the first eluate comprises an increased ratio of the full rAAV particles to the non-full rAAV particles;
(f). optionally, a second batch of the rAAV preparation in loading buffer is applied to the at least partially loaded second column, wherein the quantity of the full rAAV particles and non-full particles applied to the second column exceeds the binding capacity of the second chromatography medium such that the non-full particles bound to the second chromatography medium are displaced by the full rAAV particles into a second load flowthrough from the second column;
(g). loading the second load flowthrough form the second column to a third column comprising a third chromatography medium to obtain a loaded or partially loaded third column, preferably, the third chromatography medium is the same type as the first chromatography medium;
(h). optionally, washing the second column with a wash buffer to obtain a washed second column;
(i). bypassing the first column after step (k) or after the wash step (1) if the wash step (1) is performed, and eluting the full rAAV particles bound to the second chromatography medium with an elution buffer to obtain a second eluate and an eluted second column, wherein the second eluate comprises an increased ratio of the full rAAV particles to the non-full rAAV particles;
(j). optionally, if the third column is not saturated (i.e. loaded) after the second column flowthrough, a third batch of the rAAV preparation in loading buffer can be applied to the partially loaded third column, wherein the quantity of the full rAAV particles and non-full particles applied to the third column exceeds the binding capacity of the third chromatography medium such that the non-full particles bound to the third chromatography medium are displaced by the full rAAV particles into a third load flowthrough from the third column;
(k). if step (n) is performed, loading the third load flowthrough from the third column to the washed first column between steps (d) and (e) to obtain a second loaded first column, and optionally washing second loaded first column before continuing to elute in step (e);
(l). optionally, washing the third column with a wash buffer to obtain a washed third column;
(m). bypassing the first column and second column, and eluting the full rAAV particles bound to the third chromatography medium with an elution buffer to obtain a third eluate and an eluted third column, wherein the third eluate comprises an increased ratio of the full rAAV particles to the non-full rAAV particles; and
(n). combining the first eluate, second eluate, and third eluate to produce a purified preparation of full rAAV particles.

10. The method according to claim 9, wherein the first chromatography medium and/or the second chromatography medium and/or the third chromatography medium is an ion exchange column chromatography medium, preferably an anion exchange chromatography medium.

11. The method according to claim 9 or 10, wherein the steps (b) to (m) are performed with one or more cycles.

12. The method according to any one of claims 9-11, wherein the second column is partially loaded after step (c).

13. The method according to any one of claims 9-12, wherein the third column is partially loaded after step (g).

14. The method according to any one of claims 9-13, wherein the first column chromatography medium is selected from the group consisting of Poros 50 HQ, Poros 50 D, Poros 50 PI, Capto ImpRes Q, CIMmultus™ QA Monolithic Column and Poros XQ, preferably Poros XQ.

15. The method according to any one of claims 1-14, wherein the non-full particles comprise empty particles and/or partial particles, preferably comprise both empty particles and partial particles.

16. The method according to any one of claims 1-15, wherein the loading buffer comprises at least one buffer selected from the group consisting of Tris, Bis-tris, Bis-tris propane, Tris acetate, ethanolamine, and phosphate.

17. The method according to any one of claims 1-16, wherein the loading buffer comprises at least one salt of a cation selected from the group consisting of K(I), Li(I), Ca(II), Mg(II), Cu(II), Ba(II)), Co(II), Ni(II), Mn(II), Zn(II), Cd(II), Pb(II), Fe(III), Fe(II), Na(I), and NH₄⁺.

18. The method according to any one of claims 1-17, wherein the loading buffer comprises about 10-100 mM of a sodium salt, preferably 20-60 mM NaCl.

19. The method according to any one of claims 1-17, wherein the loading buffer comprises about 0-20 mM of a magnesium salt, preferably 1-10 mM MgCl₂.

20. The method according to any one of claims 1-17, wherein the loading buffer comprises about 0-10 mM of a calcium salt, preferably 0.1-2.5 mM CaCl₂).

21. The method according to any one of claims 1-17, wherein the loading buffer comprises about 0-100 mM of a lithium salt, preferably 0-75 mM LiCl.

22. The method according to any one of claims 1-17, wherein the loading buffer comprises about 0-5 mM of a copper salt, preferably 0.1-2.5 mM CuCl₂.

23. The method according to any one of claims 1-17, wherein the loading buffer comprises about 5-100 mM of an ammonium salt, preferably 10-50 mM (NH₄)₂SO₄.

24. The method according to any one of claims 1-23, wherein the loading buffer comprises about 20-60 mM NaCl, 20-50 mM Tris, 1-5 mM MgCl₂, 0-75 mM LiCl, 0-10 mM CuCl₂ and/or 0.1-2.5 mM CaCl₂).

25. The method according to any one of claims 1-23, the loading buffer comprises about 20-60 mM NaCl, 10-30 mM (NH₄)₂SO₄ 1-5 mM MgCl₂, 0.1-2.5 mM CuCl₂, preferably in Tris, more preferably in 20-100 mM Tris.

26. The method according to any one of claims 1-25, wherein the loading buffer has a pH of about 6-10.

27. The method according to any one of claims 1-26, wherein the loading buffer comprises at least one surfactant, selected from the group consisting of poloxamer 188, polysorbate 80, polysorbate 20, NP-40, Triton X-100, and Triton CG-110.

28. The method according to claim 27, wherein the concentration of the surfactant in the loading buffer is between 0.0001% to 0.1%.

29. The method according to any one of claims 1-28, wherein the elution buffer comprises at least one salt of a cation selected from the group consisting of K(I), Li(I), Ca(II), Mg(II), Cu(II), Ba(II)), Co(II), Ni(II), Mn(II), Zn(II), Cd(II), Pb(II), Fe(III), Fe(II), Na(I), and NH₄⁺.

30. The method according to any one of claims 1-29, wherein the elution buffer comprises about 0-1000 mM NaCl, preferably 20-300 mM NaCl.

31. The method according to any one of claims 1-29, wherein the elution buffer comprises about 0-30 mM MgCl₂, preferably 2-15 mM MgCl₂.

32. The method according to any one of claims 1-29, wherein the elution buffer comprises about 0-200 mM LiCl, preferably 0-150 mM LiCl.

33. The method according to any one of claims 1-29, wherein the elution buffer comprises about 0.1-20 mM CaCl₂), preferably 5-10 mM CaCl₂).

34. The method according to any one of claims 1-29, wherein the elution buffer comprises about 0-10 mM CuCl₂, preferably 0-3 mM CuCl₂.

35. The method according to any one of claims 1-29, wherein the elution buffer comprises about 5-100 mM of an ammonium salt, preferably 10-50 mM (NH₄)₂SO₄.

36. The method according to any one of claims 1-35, wherein the elution buffer comprises at least one buffer selected from the group consisting of Tris, Bis-tris, Bis-tris propane, Tris acetate, ethanolamine, and phosphate.

37. The method according to any one of claims 1-36, wherein the elution buffer comprises about 20-200 mM NaCl, preferably in Tris buffer.

38. The method according to any one of claims 1-37, wherein the elution buffer comprises at least one surfactant, selected from the group consisting of poloxamer 188, polysorbate 80, polysorbate 20, NP-40, Triton X-100, and Triton CG-110.

39. The method according to claim 38, wherein the concentration of the surfactant in the elution buffer is between 0.0001% to 0.1%.

40. The method according to any one of claims 1-39, wherein the elution buffer has a pH of about 6-10, preferably 8-9.

41. The method according to claim 9 or 10, wherein impurities in the rAAV preparation bind the first column with greater affinity than the full rAAV particles.

42. A method for purifying full recombinant adeno-associated virus (rAAV) particles comprising:
(a). providing an rAAV preparation comprising the full rAAV particles and non-full particles;
(b). loading the rAAV preparation in a loading buffer to a column comprising a chromatography medium, wherein the loading buffer comprises CaCl₂), and the full rAAV particles bind to the chromatography medium; and
(c). eluting the full rAAV particles bound to the chromatography medium with an elution buffer to obtain a purified preparation, wherein the elution buffer comprises CaCl₂).

43. The method according to claim 42, wherein the chromatography medium is an ion exchange column chromatography medium, preferably an anion exchange chromatography medium.

44. The method according to claim 43, wherein the column chromatography medium is selected from the group consisting of Poros 50 HQ, Poros 50 D, Poros 50 PI, Capto ImpRes Q, CIMmultus™ QA Monolithic Column, and Poros XQ, preferably Poros XQ.

45. The method according to any one of claims 42-44, wherein the loading buffer comprises at least one salt of a cation selected from the group consisting of K(I), Li(I), Ca(II), Mg(II), Cu(II), Ba(II)), Co(II), Ni(II), Mn(II), Zn(II), Cd(II), Pb(II), Fe(III), Fe(II), Na(I), and NH₄⁺.

46. The method according to any one of claims 42-45, wherein the loading buffer comprises about 0-10 mM CaCl₂), preferably 0.1-2.5 mM CaCl₂).

47. The method according to any one of claims 42-45, wherein the loading buffer comprises about 10-100 mM NaCl, preferably 20-60 mM NaCl.

48. The method according to any one of claims 42-45, wherein the loading buffer comprises about 0-20 mM MgCl₂, preferably 1-10 mM MgCl₂.

49. The method according to any one of claims 42-45, wherein the loading buffer comprises about 0-100 mM LiCl, preferably 0-75 mM LiCl.

50. The method according to any one of claims 42-45, wherein the loading buffer comprises about 0-5 mM of a copper salt, preferably 0.1-2.5 mM CuCl₂.

51. The method according to any one of claims 42-45, wherein the loading buffer comprises about 5-100 mM of an ammonium salt, preferably 10-50 mM (NH₄)₂SO₄.

52. The method according to any one of claims 42-51, wherein the loading buffer comprises at least one buffer selected from the group consisting of Tris, Bis-tris, Bis-tris propane, Tris acetate, ethanolamine, and phosphate.

53. The method according to any one of claims 42-52, wherein the loading buffer comprises about 20-60 mM NaCl, 1-5 mM MgCl₂, 0-75 mM LiCl, 0.1-2.5 mM CaCl₂), preferably in 20-50 mM Tris.

54. The method according to any one of claims 42-52, the loading buffer comprises about 20-60 mM NaCl, 10-30 mM (NH₄)₂SO₄ 1-5 mM MgCl₂, 0.1-2.5 mM CuCl₂, preferably in Tris, more preferably in 20-100 mM Tris.

55. The method according to any one of claims 42-54, wherein the loading buffer comprises at least one surfactant, selected from the group consisting of poloxamer 188, polysorbate 80, polysorbate 20, NP-40, Triton X-100, and Triton CG-110.

56. The method according to claim 55, wherein the concentration of the surfactant in the loading buffer is between 0.0001% to 0.1%.

57. The method according to any one of claims 42-56, wherein the loading buffer has a pH of about 6-10, preferably 8-9.

58. The method according to any one of claims 42-57, wherein the elution buffer comprises at least one salt of a cation selected from the group consisting of K(I), Li(I), Ca(II), Mg(II), Cu(II), Ba(II)), Co(II), Ni(II), Mn(II), Zn(II), Cd(II), Pb(II), Fe(III), Fe(II), Na(I), and NH₄⁺.

59. The method according to any one of claims 42-58, wherein the elution buffer comprises about 0.1-20 mM CaCl₂), preferably 5-10 mM CaCl₂).

60. The method according to any one of claims 42-58, wherein the elution buffer comprises about 0-1000 mM NaCl, preferably 20-300 mM NaCl.

61. The method according to any one of claims 42-58, wherein the elution buffer comprises about 0-30 mM MgCl₂, preferably 2-15 mM MgCl₂.

62. The method according to any one of claims 42-58, wherein the elution buffer comprises about 0-200 mM LiCl, preferably 0-150 mM LiCl.

63. The method according to any one of claims 42-62, wherein the elution buffer comprises at least one buffer selected from the group consisting of Tris, Bis-tris, Bis-tris propane, Tris acetate, ethanolamine, and phosphate.

64. The method according to any one of claims 42-63, wherein the elution buffer comprises about 20-200 mM NaCl, preferably in 40-60 mM Tris buffer.

65. The method according to any one of claims 42-64, wherein the elution buffer comprises at least one surfactant, selected from the group consisting of poloxamer 188, polysorbate 80, polysorbate 20, NP-40, Triton X-100, and Triton CG-110.

66. The method according to claim 65, wherein the concentration of the surfactant in the elution buffer is between 0.0001% to 0.1%.

67. The method according to any one of claims 42-66, wherein the elution buffer has a pH of about 6-10, preferably 8-9.

68. The method of any one of claims 1-67, wherein the yield of the purified full rAAV particles is no less than 70%, preferably no less than 80%, more preferably no less than 90%, most preferably not less than 95%.

69. The method of any one of claims 1-68, wherein the ratio of the full rAAV particles to the non-full particles in the purified preparation is no less than 9:1, preferably no less than 49:1.

70. The method of any one of claims 1-69, wherein the non-full particles comprise empty particles and/or partial particles, preferably comprise both empty particles and partial particles

71. A method for purifying partial rAAV particles, the method comprising:
(d). providing a non-full rAAV preparation comprising empty and partial particles;
(e). loading the non-full rAAV preparation in a loading buffer to a column comprising a chromatography medium, wherein the partial rAAV particles have a higher binding affinity to the chromatography medium than the empty particles; and
(f). eluting the partial rAAV particles bound to the chromatography medium with an elution buffer to obtain a purified preparation.

72. The method according to any one of claims 1-71, wherein the full rAAV particles comprise a transgene that encodes a polypeptide, or a nucleic acid selected from the group consisting of a siRNA, an antisense molecule, miRNA, a ribozyme and a shRNA.

73. A method for purifying empty recombinant adeno-associated virus (rAAV) particles comprising:
(a). providing an rAAV preparation comprising the empty rAAV particles, and at least one of full and partial rAAV particles;
(b). loading the rAAV preparation in a loading buffer to a column comprising a chromatography medium, wherein the empty rAAV particles have a higher binding affinity to the chromatography medium than the full or partial particles, and the quantity of the empty rAAV particles and the at least one of full and partial particles applied to the column exceeds the binding capacity of the chromatography medium, such that the at least one of full and partial particles bound to the chromatography medium are displaced by the empty rAAV particles into the flowthrough from the column; and
(c). eluting the empty rAAV particles bound to the chromatography medium with an elution buffer to obtain a purified preparation.

74. The method according to any one of claims 1-73 wherein the rAAV particles comprise a capsid derived from one or more AAVs selected from the group consisting of AAV1, AAV2, AAV3, AAV4, AAV5, AAV6, AAV7, AAV8, AAV9, AAV9.47, AAV9(hu14), AAV10, AAV11, AAV12, Rh8, Rh10, Rh74, AAV3B, AAV-218, LK03, RHM4-1, DJ, DJ8, NP59, Anc-80, and variants thereof.
